SEQUENCE LISTING (94 sequences)

SEQ ID NO: 1 - protein, 20 aa, Artificial Sequence
  Amino acid sequence for a 20mer building block of PAS#1
ASPAAPAPASPAAPAPSAPA

SEQ ID NO: 2 - protein, 20 aa, Artificial Sequence
  Amino acid sequence for a 20mer building block of P/A#1
AAPAAPAPAAPAAPAPAAPA

SEQ ID NO: 3 - protein, 20 aa, Artificial Sequence
  Amino acid sequence for a 20mer building block of APSA
APSAAPSAAPSAAPSAAPSA

SEQ ID NO: 4 - protein, 8 aa, Artificial Sequence
  MOD_RES 1: BLOCKED
  Amino acid sequence of the peptide used in fluorescence titration, N-terminally modified with ortho-aminobenzoic acid
APAPAAPA

SEQ ID NO: 5 - protein, 41 aa, Artificial Sequence
  MOD_RES 1: PYRROLIDONE CARBOXYLIC ACID
  Amino acid sequence for the PAS#1(40) polymer used for the immunogen, N-terminally modified with Pga, C-terminally modified with Ahx
  MOD_RES 41: BLOCKED
EASPAAPAPASPAAPAPSAPAASPAAPAPASPAAPAPSAPA

SEQ ID NO: 6 - protein, 41 aa, Artificial Sequence
  MOD_RES 1: PYRROLIDONE CARBOXYLIC ACID
  Amino acid sequence for the P/A#1(40) polymer used for the immunogen, N-terminally modified with Pga, C-terminally modified with Ahx
  MOD_RES 41: BLOCKED
EAAPAAPAPAAPAAPAPAAPAAAPAAPAPAAPAAPAPAAPA

SEQ ID NO: 7 - protein, 41 aa, Artificial Sequence
  MOD_RES 1: PYRROLIDONE CARBOXYLIC ACID
  Amino acid sequence for the APSA(40) polymer used for the immunogen, N-terminally modified with Pga, C-terminally modified with Ahx
  MOD_RES 41: BLOCKED
EAPSAAPSAAPSAAPSAAPSAAPSAAPSAAPSAAPSAAPSA

SEQ ID NO: 8 - protein, 6 aa, Artificial Sequence
  Amino acid sequence for epitope PAPAAP
PAPAAP

SEQ ID NO: 9 - protein, 6 aa, Artificial Sequence
  Amino acid sequence for epitope PAPASP
PAPASP

SEQ ID NO: 10 - protein, 7 aa, Artificial Sequence
  Amino acid sequence for epitope PASPAAP
PASPAAP

SEQ ID NO: 11 - protein, 120 aa, Mus musculus
  Amino acid sequence of anti-PA(S) MAb 1.1 heavy chain variable region
QVQLKESGPGLVAPSQSLSITCTVSGFSLTGYGVNWVRQPPGKGLEWLGMIWGDGITDYNSALKSRLSISKDNSKSQVFLKMNSLQTDDTARYYCARDYYGRRYYAMDYWGQGTSVTVSS

SEQ ID NO: 12 - protein, 112 aa, Mus musculus
  Amino acid sequence of anti-PA(S) MAb 1.1 light chain variable region
DIVLTQSPASLAVSLGQRATISCRASKSVSSSGYNYMFWYQQKPGQPPKLLIYLASNLESGVPDRFWGSGSGTDFTLNIHPVEEEDAATYYCQHSRELPLTFGAGTKLELKR

SEQ ID NO: 13 - protein, 123 aa, Mus musculus
  Amino acid sequence of anti-PA(S) MAb 1.2 heavy chain variable region
QVQLVESGGGLVQPGGSLKLSCAASGFTFSSYGMSWVRQTPDKRLELVATINSNGGSTYYLDSVKGRFTISRDKAKNTLYLQMSSLKSEDTAMYYCVRGGSIYDGYDYAMDYWGQGTSVTVSS

SEQ ID NO: 14 - protein, 112 aa, Mus musculus
  Amino acid sequence of anti-PA(S) MAb 1.2 light chain variable region
DIVLTQSPASLAVSLGQRATISCRASQSVSTSSYSYMHWFQQKPGQPPKLLIKYASNLESGVPARFSGSGSGTDFTLNIHPVEEEDTATYYCQHSWEIPLTFGAGTKLELKR

SEQ ID NO: 15 - protein, 121 aa, Mus musculus
  Amino acid sequence of anti-PA(S) MAb 2.1 heavy chain variable region
QVTLKESGPGILQPSQTLSLTCSFSGFSLSTYDMGVGWIRQPSGKGLEWLANIWWNDNKYYNSALKSRLTISKDTSNNQVFLKISSVDATDTATYYCAQMRDNYGVWFAFWGQGTLVTVSA

SEQ ID NO: 16 - protein, 113 aa, Mus musculus
  Amino acid sequence of anti-PA(S) MAb 2.1 light chain variable region
DIVMSQSPSSLAVSVGEKVTMSCKSSQSLFYSNREKNYLAWYQQKPGQSPKLLIYWASTRESGVPDRFTGSGSGTDFTLTISNVKAEDLAVYYCQQYYNYPTFGAGTKLELKR

SEQ ID NO: 17 - protein, 121 aa, Mus musculus
  Amino acid sequence of anti-PA(S) MAb 2.2 heavy chain variable region
QVTLKESGPGILQPSQTLSLTCSFSGFSLNTYGMGVGWIRQPSGKGLEWLANIWWTDDKYYNSVLKSRLTISKDTFNNQVFLKISSVDTADTATYYCAQLAYHDNPWFAYWGQGTLVTVSA

SEQ ID NO: 18 - protein, 113 aa, Mus musculus
  Amino acid sequence of anti-PA(S) MAb 2.2 light chain variable region
DIVMSQSPSSLAVSVGEKVTLSCKSSQSLLYSNNQKNYLAWYQQKPGQSPKLLIYWASTRESGVPDRFTGSGSGTDFTLTISSVKAEDLAVYYCQQYYNYPTFGGGTKLEIKR

SEQ ID NO: 19 - protein, 112 aa, Mus musculus
  Amino acid sequence of anti-PA(S) MAb 3.1 heavy chain variable region
QVQLQQSGAELVRPGASVKLSCKALGYIFTDYEMHWVKQTPVHGLEWIGVIHPGSGNTVYNQKFQGKATLTADKSSSTAYMEVSSLTSEDSAVYYCNLWGRWGQGTTLTVSS

SEQ ID NO: 20 - protein, 113 aa, Mus musculus
  Amino acid sequence of anti-PA(S) MAb 3.1 light chain variable region
DVVMTQTPVTLSVNIGQPASISCKSGQSLLHSDGKTYLNWLLQRPGQSPKRLIYLVSDLDSGVPDRFTSSGSGTDFTLEISRVEAEDLGVYYCWQGTHLPHTFGGGTKLEIKR

SEQ ID NO: 21 - protein, 114 aa, Mus musculus
  Amino acid sequence of anti-PA(S) MAb 3.2 heavy chain variable region
QVQLQQSGAELVRPGASVKLSCKALGYTFTDYEMFWVKQTPVHGLECIGTIHPGSGGTAYNQKFKGKATLTADKSSSTAYMELSSLTSEDSAVYYCTRNDRGSWGQGTTLTVSS

SEQ ID NO: 22 - protein, 113 aa, Mus musculus
  Amino acid sequence of anti-PA(S) MAb 3.2 light chain variable region
DVVLTQTPLTLSVTIGQPASISCRSSQSLLNSDGKTFLNWLLQRPGLSPKRLIYLVSKLDSGVPDRFTGSGSGTDFTLKISRVEAEDLGVYYCWQGTHFPHTFGGGTKLEIKR

SEQ ID NO: 23 - DNA, 360 bp, Mus musculus
  Nucleotide sequence of anti-PA(S) MAb 1.1 heavy chain variable region
caggtgcagctgaaggagtcaggacctggcctggtggcgccctcacagagcctgtccatcacatgcaccgtctcagggttctcattaaccggctatggtgtaaattgggttcgccagcctccaggaaagggtctggagtggctgggaatgatatggggtgatggaatcacagactataattcagctctcaaatccagactgagcatcagcaaggacaactccaagagtcaagttttcttaaaaatgaacagtctgcaaactgatgacacagccaggtactactgtgccagggattactacggtaggaggtactatgctatggactactggggtcaaggaacctcagtcaccgtctcctca

SEQ ID NO: 24 - DNA, 336 bp, Mus musculus
  Nucleotide sequence of anti-PA(S) MAb 1.1 light chain variable region
gacattgtactgacacagtctcctgcttccttagctgtatctctggggcagagggccaccatctcatgcagggccagcaaaagtgtcagttcatctggctataattatatgttctggtaccaacagaaaccaggacagccacccaaactcctcatctatcttgcatccaacctagaatctggggtccctgacaggttctggggcagtgggtctgggacagacttcaccctcaacatccatcctgtggaggaggaggatgcagcaacctattactgtcagcacagtagggagcttcctctaacgttcggtgctgggaccaagctggagctgaaacgg

SEQ ID NO: 25 - DNA, 369 bp, Mus musculus
  Nucleotide sequence of anti-PA(S) MAb 1.2 heavy chain variable region
gaggtgcagctggtggagtctgggggaggcttagtgcagcctggagggtccctgaaactctcctgtgcagcctctggattcactttcagtagctatggcatgtcttgggttcgccagactccagacaagaggctggagttggtcgcaaccattaatagtaatggtggtagcacctattatctagacagtgtgaagggccgattcaccatctccagagacaaagccaagaacaccctgtacctgcaaatgagcagtctgaagtctgaggacacagccatgtattactgtgtaagaggggggtcgatctatgatggttacgactatgctatggactactggggtcaaggaacctcagtcaccgtctcctca

SEQ ID NO: 26 - DNA, 336 bp, Mus musculus
  Nucleotide sequence of anti-PA(S) MAb 1.2 light chain variable region
gacattgtgctgacacagtctcctgcttccttagctgtatctctggggcagagggccaccatctcatgcagggccagccaaagtgtcagtacatctagctatagttatatgcactggttccaacagaaaccaggacagccacccaaactcctcatcaagtatgcatccaacctagagtctggggtccctgccaggttcagtggcagtgggtctgggacagacttcaccctcaacatccatcctgtggaggaggaggatactgcaacatattactgtcagcacagttgggagattcctctcacgttcggtgctgggaccaagctggagctgaaacgg

SEQ ID NO: 27 - DNA, 363 bp, Mus musculus
  Nucleotide sequence of anti-PA(S) MAb 2.1 heavy chain variable region
caggtcactctgaaagagtctggccctgggatattgcagccctcccagaccctcagtctgacttgttctttctctgggttttcactgagcacttatgatatgggtgtaggttggattcgtcagccttcagggaagggtctggagtggctggccaacatttggtggaatgataataaatactataactcagccctgaagagccgcctcacaatctccaaggatacctccaacaaccaggtgttcctcaagatctccagtgtggacgctacagatactgccacatattattgtgctcaaatgagggataactacggagtttggtttgctttctggggccaagggactctggtcactgtctctgca

SEQ ID NO: 28 - DNA, 339 bp, Mus musculus
  Nucleotide sequence of anti-PA(S) MAb 2.1 light chain variable region
gacattgtgatgtcacagtctccatcctccctagctgtgtcagttggagagaaggttactatgagctgcaagtccagtcagagccttttttatagtaacagggaaaagaactacttggcctggtaccagcagaaaccagggcagtctcctaaactgctgatttactgggcatccactagggaatctggggtccctgatcgcttcacaggcagtggatctgggacagatttcactctcaccatcagcaatgtgaaggctgaagacctggcagtttattactgtcagcaatattataactatcccacgttcggtgctgggaccaagctggagctgaaacgg

SEQ ID NO: 29 - DNA, 363 bp, Mus musculus
  Nucleotide sequence of anti-PA(S) MAb 2.2 heavy chain variable region
caggtcactctgaaagagtctggccctgggatattgcagccctcccagaccctcagtctgacttgttctttctctgggttttcactgaacacttatggtatgggtgtaggttggattcgtcagccttcagggaagggtctggagtggctggccaacatttggtggactgatgataagtactataattcagtcctgaagagccggctcacaatctccaaggataccttcaacaaccaggtattcctcaagatctccagtgtggacactgcagatactgccacatactactgtgctcaactagcctatcatgataacccctggtttgcttactggggccaagggactctggtcactgtctctgca

SEQ ID NO: 30 - DNA, 339 bp, Mus musculus
  Nucleotide sequence of anti-PA(S) MAb 2.2 light chain variable region
gacattgtgatgtcacagtctccatcctccctagctgtgtcagttggagagaaggttactctgagctgcaagtccagtcagagccttttatatagtaacaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattataactatcccacgttcggaggggggaccaagctggaaataaaacgg

SEQ ID NO: 31 - DNA, 336 bp, Mus musculus
  Nucleotide sequence of anti-PA(S) MAb 3.1 heavy chain variable region
caggttcaactgcagcagtctggggctgagctggtgaggcctggggcttcagtgaagctgtcctgcaaggctttgggctacatatttactgactatgaaatgcactgggtgaagcagacacctgtacatggcctggaatggattggagttattcatccaggaagtggtaatactgtctacaatcagaagttccagggcaaggccacactgactgcagacaaatcctccagcacagcctacatggaggtcagcagcctgacatctgaggactctgctgtctattactgtaatctatggggaaggtggggccaaggcaccactctcacagtctcctca

SEQ ID NO: 32 - DNA, 339 bp, Mus musculus
  Nucleotide sequence of anti-PA(S) MAb 3.1 light chain variable region
gatgttgtgatgacccagactccagtcactttgtcggttaacattggacaaccagcctccatctcttgcaagtcaggtcagagcctcttacatagtgatggaaagacatatttgaattggttgttacagaggccaggccagtctccaaagcgcctaatctatctggtgtctgatctggactctggagtccctgacaggttcactagcagtggatcagggacagatttcacactggaaatcagcagagtggaggctgaggatttgggagtttattattgctggcaaggtacacatcttccgcatacgttcggaggggggaccaagctggaaataaaacgg

SEQ ID NO: 33 - DNA, 342 bp, Mus musculus
  Nucleotide sequence of anti-PA(S) MAb 3.2 heavy chain variable region
caggttcaactgcagcagtctggggctgagctggtgaggcctggggcttcagtgaagctgtcctgcaaggctttgggctacacatttactgactatgaaatgttctgggtgaagcagacacctgtgcatggcctggaatgtattggaactattcatccaggaagtggtggaactgcctacaatcagaagttcaagggcaaggccacactgactgcagacaaatcctccagcacagcctacatggagctcagcagcctgacgtctgaggactctgctgtctattactgtacaagaaatgacagaggctcctggggccaaggcaccactctcacagtctcctca

SEQ ID NO: 34 - DNA, 339 bp, Mus musculus
  Nucleotide sequence of anti-PA(S) MAb 3.2 light chain variable region
gatgttgtgctgacccagactccactcactttgtcggttaccattggacaaccagcctccatctcttgcaggtcaagtcagagcctcttaaatagtgatgggaagacatttttgaattggttgttacagaggccaggcctgtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcacacgttcggaggggggaccaagctggaaataaaacgg

SEQ ID NO: 35 - protein, 10 aa, Mus musculus
  Amino acid sequence of anti-PA(S) MAb 1.1 CDRH1
GFSLTGYGVN

SEQ ID NO: 36 - protein, 16 aa, Mus musculus
  Amino acid sequence of anti-PA(S) MAb 1.1 CDRH2
MIWGDGITDYNSALKS

SEQ ID NO: 37 - protein, 12 aa, Mus musculus
  Amino acid sequence of anti-PA(S) MAb 1.1 CDRH3
DYYGRRYYAMDY

SEQ ID NO: 38 - protein, 15 aa, Mus musculus
  Amino acid sequence of anti-PA(S) MAb 1.1 CDRL1
RASKSVSSSGYNYMF

SEQ ID NO: 39 - protein, 7 aa, Mus musculus
  Amino acid sequence of anti-PA(S) MAb 1.1 CDRL2
LASNLES

SEQ ID NO: 40 - protein, 9 aa, Mus musculus
  Amino acid sequence of anti-PA(S) MAb 1.1 CDRL3
QHSRELPLT

SEQ ID NO: 41 - protein, 10 aa, Mus musculus
  Amino acid sequence of anti-PA(S) MAb 1.2 CDRH1
GFTFSSYGMS

SEQ ID NO: 42 - protein, 17 aa, Mus musculus
  Amino acid sequence of anti-PA(S) MAb 1.2 CDRH2
TINSNGGSTYYLDSVKG

SEQ ID NO: 43 - protein, 14 aa, Mus musculus
  Amino acid sequence of anti-PA(S) MAb 1.2 CDRH3
GGSIYDGYDYAMDY

SEQ ID NO: 44 - protein, 15 aa, Mus musculus
  Amino acid sequence of anti-PA(S) MAb 1.2 CDRL1
RASQSVSTSSYSYMH

SEQ ID NO: 45 - protein, 7 aa, Mus musculus
  Amino acid sequence of anti-PA(S) MAb 1.2 CDRL2
YASNLES

SEQ ID NO: 46 - protein, 9 aa, Mus musculus
  Amino acid sequence of anti-PA(S) MAb 1.2 CDRL3
QHSWEIPLT

SEQ ID NO: 47 - protein, 12 aa, Mus musculus
  Amino acid sequence of anti-PA(S) MAb 2.1 CDRH1
GFSLSTYDMGVG

SEQ ID NO: 48 - protein, 16 aa, Mus musculus
  Amino acid sequence of anti-PA(S) MAb 2.1 CDRH2
NIWWNDNKYYNSALKS

SEQ ID NO: 49 - protein, 11 aa, Mus musculus
  Amino acid sequence of anti-PA(S) MAb 2.1 CDRH3
MRDNYGVWFAF

SEQ ID NO: 50 - protein, 17 aa, Mus musculus
  Amino acid sequence of anti-PA(S) MAb 2.1 CDRL1
KSSQSLFYSNREKNYLA

SEQ ID NO: 51 - protein, 7 aa, Mus musculus
  Amino acid sequence of anti-PA(S) MAb 2.1 CDRL2
WASTRES

SEQ ID NO: 52 - protein, 8 aa, Mus musculus
  Amino acid sequence of anti-PA(S) MAb 2.1 CDRL3
QQYYNYPT

SEQ ID NO: 53 - protein, 12 aa, Mus musculus
  Amino acid sequence of anti-PA(S) MAb 2.2 CDRH1
GFSLNTYGMGVG

SEQ ID NO: 54 - protein, 16 aa, Mus musculus
  Amino acid sequence of anti-PA(S) MAb 2.2 CDRH2
NIWWTDDKYYNSVLKS

SEQ ID NO: 55 - protein, 11 aa, Mus musculus
  Amino acid sequence of anti-PA(S) MAb 2.2 CDRH3
LAYHDNPWFAY

SEQ ID NO: 56 - protein, 17 aa, Mus musculus
  Amino acid sequence of anti-PA(S) MAb 2.2 CDRL1
KSSQSLLYSNNQKNYLA

SEQ ID NO: 57 - protein, 7 aa, Mus musculus
  Amino acid sequence of anti-PA(S) MAb 2.2 CDRL2
WASTRES

SEQ ID NO: 58 - protein, 8 aa, Mus musculus
  Amino acid sequence of anti-PA(S) MAb 2.2 CDRL3
QQYYNYPT

SEQ ID NO: 59 - protein, 10 aa, Mus musculus
  Amino acid sequence of anti-PA(S) MAb 3.1 CDRH1
GYIFTDYEMH

SEQ ID NO: 60 - protein, 17 aa, Mus musculus
  Amino acid sequence of anti-PA(S) MAb 3.1 CDRH2
VIHPGSGNTVYNQKFQG

SEQ ID NO: 61 - skipped

SEQ ID NO: 62 - protein, 16 aa, Mus musculus
  Amino acid sequence of anti-PA(S) MAb 3.1 CDRL1
KSGQSLLHSDGKTYLN

SEQ ID NO: 63 - protein, 7 aa, Mus musculus
  Amino acid sequence of anti-PA(S) MAb 3.1 CDRL2
LVSDLDS

SEQ ID NO: 64 - protein, 9 aa, Mus musculus
  Amino acid sequence of anti-PA(S) MAb 3.1 CDRL3
WQGTHLPHT

SEQ ID NO: 65 - protein, 10 aa, Mus musculus
  Amino acid sequence of anti-PA(S) MAb 3.2 CDRH1
GYTFTDYEMF

SEQ ID NO: 66 - protein, 17 aa, Mus musculus
  Amino acid sequence of anti-PA(S) MAb 3.2 CDRH2
TIHPGSGGTAYNQKFKG

SEQ ID NO: 67 - protein, 5 aa, Mus musculus
  Amino acid sequence of anti-PA(S) MAb 3.2 CDRH3
NDRGS

SEQ ID NO: 68 - protein, 16 aa, Mus musculus
  Amino acid sequence of anti-PA(S) MAb 3.2 CDRL1
RSSQSLLNSDGKTFLN

SEQ ID NO: 69 - protein, 7 aa, Mus musculus
  Amino acid sequence of anti-PA(S) MAb 3.2 CDRL2
LVSKLDS

SEQ ID NO: 70 - protein, 9 aa, Mus musculus
  Amino acid sequence of anti-PA(S) MAb 3.2 CDRL3
WQGTHFPHT

SEQ ID NO: 71 - protein, 450 aa, Artificial Sequence
  Amino acid sequence of StrepII-eGFP-PAS#1(200)
ASWSHPQFEKGASKGEELFTGVVPILVELDGDVNGHKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYQASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAA

SEQ ID NO: 72 - protein, 364 aa, Artificial Sequence
  Amino acid sequence of PAS#1(200)-IL1Ra
MKHHHHHHSSSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAARPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 73 - protein, 364 aa, Artificial Sequence
  Amino acid sequence of P/A#1(200)-IL1Ra
MKHHHHHHSSSAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAARPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 74 - protein, 353 aa, Artificial Sequence
  Amino acid sequence of APSA(200)-IL1Ra
APSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAAPSAARPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 75 - protein, 736 aa, Artificial Sequence
  Amino acid sequence of P/A#1(600)-GMCSF
HHHHHHSSSAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAAPAAPAPAAPAAPAPAAPAAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 76 - protein, 629 aa, Artificial Sequence
  MOD_RES 1: ACETYLATION
  Amino acid sequence of PASylated Thymosin-alpha1, N-terminally acetylated
SDAAVDTSSEITTKDLKEKKEVVEEAENASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAA

SEQ ID NO: 77 - DNA, 18 bp, Mus musculus
  Nucleotide sequence of Primer RMK
gacctccacggagtcagc

SEQ ID NO: 78 - DNA, 18 bp, Mus musculus
  Nucleotide sequence of Primer RMG
aggtcgccacacgtgtgg

SEQ ID NO: 79 - protein, 6 aa, Artificial Sequence
  Amino acid sequence for epitope PSAAPS
PSAAPS

SEQ ID NO: 80 - protein, 6 aa, Artificial Sequence
  Amino acid sequence for epitope ASPAAP
ASPAAP

SEQ ID NO: 81 - protein, 6 aa, Artificial Sequence
  Amino acid sequence for epitope PASPAA
PASPAA

SEQ ID NO: 82 - protein, 4 aa, Artificial Sequence
  Amino acid sequence for epitope PAAP
PAAP

SEQ ID NO: 83 - protein, 4 aa, Artificial Sequence
  Amino acid sequence for epitope PASP
PASP

SEQ ID NO: 84 - protein, 4 aa, Artificial Sequence
  Amino acid sequence for epitope APSA
APSA

SEQ ID NO: 85 - protein, 4 aa, Artificial Sequence
  Amino acid sequence for epitope PSAA
PSAA

SEQ ID NO: 86 - protein, 4 aa, Artificial Sequence
  Amino acid sequence for epitope ASPA
ASPA

SEQ ID NO: 87 - protein, 4 aa, Artificial Sequence
  Amino acid sequence for epitope APAP
APAP

SEQ ID NO: 88 - protein, 4 aa, Artificial Sequence
  Amino acid sequence for epitope SAPA
SAPA

SEQ ID NO: 89 - protein, 4 aa, Artificial Sequence
  Amino acid sequence for epitope AAPA
AAPA

SEQ ID NO: 90 - protein, 388 aa, Artificial Sequence
  H1GA-PAS#1(200)-His6
QDSTSDLIPAPPLSKVPLQQNFQDNQFHGKWYVVGAAGNVLLREDKDPLKMYATIYELKEDKSYNVTSVGFDDKKCLYKIRTFVPGSQPGEFTLGRIKSEPGGTSWLVRVVSTNYNQHAMVFFKEVAQNRETFNITLYGRTKELTSELKENFIRFSKSLGLPENHIVFPVPIDQCIDGSSASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPASAHHHHHH

SEQ ID NO: 91 - protein, 954 aa, Artificial Sequence
  PAS#1(800)-IL1Ra
PASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAARPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 92 - protein, 227 aa, Artificial Sequence
  Heavy chain PASylated humanized anti-Galectin-3 Fab
QIQLVQSGAEVKKPGESLKISCKVSGYTFTDYAIHWVRQMPGKGLKWMGWINTYTGKPIYADDFKGQVIISLDKSINTAYLQWSSLKASDTAIYYCARGTMMASLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 93 - protein, 420 aa, Artificial Sequence
  Light chain PASylated humanized anti-Galectin-3 Fab
DVVMTQSPSSLSASVGDRVTITCKSSQSLVHSDGKTYLSWIQQKPGKAPKGLIYLVSKLDSGVPSRFSGSGSETDFTLTISSLQPEDSATYYCWQATHFPLTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAA

SEQ ID NO: 94 - protein, 259 aa, Artificial Sequence
  Human Galectin-3 C173T C-terminally fused with Strep-tagII
ADNFSLHDALSGSGNPNPQGWPGAWGNQPAGAGGYPGASYPGAYPGQAPPGAYPGQAPPGAYPGAPGAYPGAPAPGVYPGPPSGPGAYPSSGQPSATGAYPATGPYGAPAGPLIVPYNLPLPGGVVPRMLITILGTVKPNANRIALDFQRGNDVAFHFNPRFNENNRRVIVTNTKLDNNWGREERQSVFPFESGKPFKIQVLVEPDHFKVAVNDAHLLQYNHRVKKLNEISKLGISGDIDLTSASYTMISAWSHPQFEK

(19) United States
(12) Patent Application Publication
(10) Pub. No.: US 2024/0294632 A1
(43) Pub. Date: Sep. 5, 2024

(54) ANTIBODIES SPECIFIC FOR STRUCTURALLY DISORDERED SEQUENCES

(71) Applicants: XL-PROTEIN GMBH, Freising (DE); TECHNISCHE UNIVERSITÄT MÜNCHEN, München (DE)

(72) Inventors: Jonas SCHILZ, Freising (DE); Uli BINDER, Freising (DE); Lars FRIEDRICH, München (DE); Michaela GEBAUER, Dachau (DE); Martin SCHLAPSCHY, Freising (DE); Arne SKERRA, Dachau (DE)

(73) Assignees: XL-PROTEIN GMBH, Freising (DE); TECHNISCHE UNIVERSITÄT MÜNCHEN, München (DE)

(21) Appl. No.: 18/268,880

(22) PCT Filed: Dec. 22, 2021
(86) PCT No.: PCT/EP2021/087365

§ 371 Date: Jun. 21, 2023

(30) Foreign Application Priority Data
Dec. 22, 2020 (EP) 20216744.1

(51) Int. Cl. C07K 16/26; C07K 14/00; C07K 14/575; C07K 16/44

(52) U.S. Cl. CPC C07K 16/26; C07K 14/001; C07K 14/57581; C07K 16/44; C07K 2317/33; C07K 2317/35; C07K 2317/55; C07K 2317/92

(57) ABSTRACT

The present invention relates to a method for generating and/or obtaining specific binding moieties against intrinsically disordered proteins (IDPs) and/or intrinsically disordered protein domains which tend to be immunologically inert and lack immunogenicity in animals, in particular in mammals. The present invention also relates to such specific binding moieties, in particular to antibodies and/or to antigen binding fragments thereof, specifically binding to structurally disordered and/or intrinsically disordered sequences, in particular to Pro/Ala-rich sequences (PAS). These binding moieties, antibodies, antigen binding fragments are first in class since they bind to/recognize disordered peptides or polypeptide fragments as also comprised in such “intrinsically disordered proteins”, in particular PAS polypeptides. The inventive binding moieties, antibodies, antigen binding fragments are, without being limiting, particularly useful in diagnostic settings as well as research tools. The present invention relates to a method for generating and/or obtaining specific binding moieties against intrinsically disordered proteins (IDPs) and/or intrinsically disordered protein domains which tend to be immunologically inert and lack immunogenicity in animals, in particular in mammals. The present invention also relates to such specific binding moieties, in particular to antibodies and/or to antigen binding fragments thereof, specifically binding to structurally disordered and/or intrinsically disordered sequences, in particular to Pro/Ala-rich sequences (PAS). These binding moieties, antibodies, antigen binding fragments are first in class since they bind to/recognize disordered peptides or polypeptide fragments as also comprised in such “intrinsically disordered proteins”, in particular PAS polypeptides. The inventive binding moieties, antibodies, antigen binding fragments are, without being limiting, particularly useful in diagnostic settings as well as research tools.

The present invention relates to a method for generating and/or obtaining specific binding moieties against intrinsically disordered proteins (IDPs) and/or intrinsically disordered protein domains which tend to be immunologically inert and lack immunogenicity in animals, in particular in mammals. The present invention also relates to such specific binding moieties, in particular to antibodies and/or to antigen binding fragments thereof, specifically binding to structurally disordered and/or intrinsically disordered sequences, in particular to Pro/Ala-rich sequences (PAS). These binding moieties, antibodies, antigen binding fragments are first in class since they bind to/recognize disordered peptides or polypeptide fragments as also comprised in such “intrinsically disordered proteins”, in particular PAS polypeptides. The inventive binding moieties, antibodies, antigen binding fragments are, without being limiting, particularly useful in diagnostic settings as well as research tools.

The invention relates in particular and in one specific embodiment to method(s) for generating an antigen binding molecule, preferably an antibody or an antigen-binding fragment thereof, directed against intrinsically disordered peptides/proteins and/or intrinsically disordered peptide/protein domains, said method comprising the step of immunizing a non-human mammal with an antigen, wherein said antigen is a conjugate of an immunoadjuvant and one or more P/A peptides, wherein each P/A peptide is independently a peptide consisting of about 5 to about 100 amino acid residues, wherein at least 60% of the amino acid residues of said peptide are independently selected from proline and alanine, and wherein a protecting group R^{N} is attached to the N-terminal amino group of said peptide

The present invention also relates to specific structurally defined hybridomas comprising nucleic acid sequences encoding for the inventive specific binding moieties/antibodies/antibody fragments and/or encoding for variable regions (like variable heavy chain sequences and/or variable light chain sequences) and/or complementarity determining regions (CDRs) of said inventive specific binding moieties/antibodies/antibody fragments. The present invention further relates to nucleic acid molecules encoding CDRs and/or the light chain variable region or the heavy chain variable region of the antibody of the invention as well as vectors comprising said nucleic acid molecules. The invention also relates to a host cell comprising the vector(s) of the invention as well as to methods for the production of binding moieties/antibodies/antibody fragments of the invention comprising culturing the host cell of the invention and/or a hybridoma of the invention under suitable conditions and isolating the binding moieties/antibodies/antibody fragments produced. Accordingly, the invention also relates to hybridomas and/or host cells expressing the binding moieties/antibodies/antibody fragments of the present invention.

Furthermore, the present invention relates to binding moieties/antibodies/antibody fragments obtainable by the method of the invention, to a composition comprising at least one binding moiety, antibody or antigen binding fragment of the invention, the hybridoma and/or host cell of the invention, the nucleic acid molecule of the invention, the vector of the invention, the hybridoma/host cell of the invention or the binding moiety, antibody or antigen binding fragment produced by the method of the invention. The present invention also relates to the use of a binding moiety, an antibody or antigen binding fragment of the invention for detecting, quantifying and/or discriminating intrinsically disordered proteins and/or intrinsically disordered protein domains, in particular PAS sequences and/or molecules comprising PAS sequences. Such a detection, quantification or discrimination may also be carried out on biological samples in accordance with the invention, for example on blood or plasma samples, or on samples of the cerebrospinal fluid, vitreous of the eye, tissue sections and the like. The present invention also provides for research tools and/or diagnostic reagents for the preclinical and clinical development of PASylated biologics. Also provided herein are means and methods for the screening of biological samples obtained from subjects, in particular human patients, treated with such PASylated biologics, i.e. drug conjugates comprising a biologically active (protein) drug and a Pro/Ala-rich sequence (PAS) comprising e.g. the small residues Pro, Ala and Ser or Pro and Ala only. Said drug conjugates are not limited to protein drugs or biologics may also comprise “small molecule” drugs and chemical drugs as well as carbohydrate drugs and nucleic acid drugs.

The present invention also relates to the use of a binding moiety, an antibody or antigen binding fragment of the invention for preparation of means in diagnose settings, for laboratory uses, in research and/or development including preclinical or clinical development, in purification methods etc. For example, the inventive binding moiety, an antibody or antigen binding fragment may be employed in matrix-based protein/peptide purification or immobilization. Inter alia, an affinity matrix for the purification of intrinsically disordered proteins and/or intrinsically disordered protein domains, in particular PAS sequences and/or molecules comprising PAS sequences, may be prepared with the herein provided inventive compounds, like the inventive antibodies or antigen binding fragments thereof.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Intrinsically disordered proteins or protein domains (IDPs) are common in nature and play important roles in signal transduction and protein trafficking, as in the case of synaptojanin or the transcriptional activation domain of RelA (Snead & Eliezer, 2019; Tantos et al., 2012; Wright & Dyson, 2015), for example. Such IDPs are also abundant in a wide range of pathogens and, thus, represent potential targets to combat infectious diseases (Feng et al., 2006). In contrast to the specific interactions with structured proteins, the properties of disordered peptides or polypeptide segments (as also comprised in IDPs) often pose a challenge for the immune system in the generation of cognate antibodies, a feature that is exploited by pathogens to evade the immune response (Giri et al., 2016; Goh et al., 2016).

Antigens are mostly proteins or peptides whose surface epitopes act as point of interaction for specific antibody recognition. Epitopes are generally divided into two categories, (i) linear epitopes that are defined by their primary structure and (ii) conformational epitopes, where the key amino acids are discontinuous in the amino acid sequence but brought into close proximity in the (structurally defined) three-dimensional fold (Barlow et al., 1986). It has long been assumed that epitopes are predominantly discontinuous (Barlow et al., 1986); in fact, more recent analyses suggest that conformational epitopes constitute about 90% of all B-cell epitopes present in native proteins (Huang & Honda, 2006).

While mutual interactions between disordered proteins have been analysed in detail (Fong et al., 2009; Meszaros et al., 2007; Uversky, 2019), only a few studies have addressed the structural aspects of complex formation between disordered protein antigens and structurally defined binding partners such as antibodies (Fassolari et al., 2013; MacRaild et al., 2016). In general, peptides form interfaces with antibodies that are dominated by hydrogen bonds, often involving the peptide backbone, and they tend to bind in a more planar fashion than proteins (London et al., 2010). Furthermore, IDPs present smaller epitopes than folded antigens and appear to be more efficient in terms of free energy gain per contact residue (MacRaild et al., 2016). Structural analyses of protein antigens have shown that residues in disordered epitopes are more likely involved in hydrogen bonds and salt bridges than those in conformational epitopes (MacRaild et al., 2016). More specifically, as the prior art has postulated, interfaces with peptides are normally enriched in large hydrophobic side chains, such as Phe, Leu, Trp, Tyr and lie, which serve as hot spot for binding (London et al., 2010).

In the last decade, artificial structurally disordered polypeptides have gained attention in the field of pharmaceutical biotechnology, where they are used to tailor the in vivo properties of protein fusion partners or drug conjugates as a functional substitute of polyethylene glycol (PEG), a highly hydrophilic chemical polymer (Schellenberger et al., 2009; Schlapschy et al., 2013). For example, conjugation of pharmacologically active proteins or peptides or of (“small”) molecules with long polypeptides comprising the three small amino acids Pro, Ala and/or Ser, known as PASylation®, dramatically expands the hydrodynamic volume and prolongs the plasma half-life by retarding renal filtration (Binder & Skerra, 2017). Accordingly, Pro/Ala-rich sequence (PAS) polypeptides were developed as a biological alternative to poly-ethylene glycol (PEG) to generate biopharmaceuticals with extended plasma half-life. Much like the chemical macromolecule PEG, recombinant PAS polypeptides are conformationally disordered and show high solubility in water. Indeed, devoid of any charged or pronounced hydrophobic side chains these biosynthetic polymers represent an extreme case of IDPs.

As discussed above, the PASylation® approach relies on conformationally disordered polypeptide chains with expanded hydrodynamic volume comprising Pro/Ala-rich sequences (PAS), i.e. the small residues Pro, Ala and Ser or Pro and Ala only. These PAS sequences are hydrophilic, uncharged biological polymers with biophysical properties very similar to PEG, whose chemical conjugation to drugs is an established method for plasma half-life extension. In contrast to PEG, PAS polypeptides have been described to enable the simple fusion to therapeutic proteins or peptides on the genetic level, permitting the production of fully active therapeutic proteins in E. coli or other host cells and obviating in vitro coupling or modification steps (Binder & Skerra, 2017). Furthermore, Pro/Ala-rich sequences (PAS)/PAS polypeptides are biodegradable, thus avoiding organ accumulation, while showing stability in serum and lacking toxicity. One of the further advantages of the PASylation® technology is in fact the provision of PAS polypeptides that are immunologically inert and are therefore of great advantage for medical and therapeutic use. However, the lack of immunogenicity also is the reason why no antibodies against such intrinsically disordered proteins (IDPs) and/or intrinsically disordered protein domains are described in the art. Yet, such specific antibodies are desired, in particular as research tools and in diagnostic settings, including patient stratification and/or monitoring for treatment responses.

Accordingly, the technical problem underlying the present invention is the provision of means and methods for the preparation of binding moieties, in particular antibodies and/or antibody fragments, that specifically bind intrinsically disordered proteins or protein domains, in particular disordered polypeptide chains with expanded hydrodynamic volume comprising the amino acid residues Pro and Ala and/or Pro, Ala and Ser (PAS).

This technical problem is solved by the embodiments as provided herein and in the appended claims.

In a first embodiment, the present invention relates to a method for generating an antigen binding molecule, preferably an antibody or an antigen-binding fragment thereof, directed against intrinsically disordered peptides/proteins and/or intrinsically disordered peptide/protein domains, said
method comprising the step of immunizing a non-human mammal with an antigen,
wherein said antigen is a conjugate of an immunoadjuvant and one or more P/A peptides,
wherein each P/A peptide is independently a peptide consisting of about 5 to about 100 amino acid residues, wherein at least 60% of the amino acid residues of said peptide are independently selected from proline and alanine, and wherein a protecting group R^{N} is attached to the N-terminal amino group of said peptide.

In context of the present invention and as illustrated in the examples, the inventors have surprisingly found that the PAS polypeptides, when conjugated to an immunoadjuvant/highly immunogenic carrier protein such as KLH, which preferably forms a protein complex with a molecular mass of more than about 5 megadaltons (5 MDa), in combination with the immunization scheme as disclosed herein, can elicit a PAS-directed antibody response in non-human mammals, in particular in mice. This is unexpected and is rendered possible by the means and methods as disclosed herein. It is generally believed that conjugation to an immunoadjuvant provides additional T cell epitopes and thus also increases the immunogenicity of the peptide portion of the conjugate. Hence, it is thought that the ability of the immune system is increased to generate antibodies that specifically bind to these peptides. However, it has been shown previously (Schlapschy et al., 2013) in the same BALB/c mouse strain which was used in the present invention that conjugation of the PAS moiety to a protein moiety such as human IFNα2b and repeatedly immunizing mice, even using Freund's adjuvans as an immunopotentiator (booster), did not lead to the generation of PAS specific antibodies at all but rather led to the generation of IFNα2b specific antibodies. Similarly, in plasma samples of mice that had been repeatedly treated with PAS-hGH IgG reactive against the human growth hormone (hGH) moiety was detectable on a Western Blot, but there was no cross-reactivity with the PAS sequence fused to other proteins, indicating that the PAS polypeptide itself is not immunogenic (Schlapschy et al., 2013). In fact, this complete lack of PAS-directed immunogenicity, even when fused to a protein moiety, is a key feature of the PASylation technology which can directly be attributed to the nature of the small and biochemically inert amino acids Pro, Ala and Ser, or to Pro and Ala which constitute such PA(S) peptides.

The prior art has postulated that interfaces with peptides are normally enriched in large hydrophobic side chains, such as Phe, Leu, Trp, Tyr and lie (being contained in peptides which have been used for conjugation to carrier proteins), which serve as hot spot for binding (London et al., 2010). On the contrary and thus surprisingly, the appended crystal structures of the inventive binding moieties of the present invention reveal a particularly relevant and unprecedented role of Ala in the recognition of the PA(S) peptide. This is particularly surprising, since all of the amino acids comprised in PA(S) peptides have immunologically/chemically inert side chains and are devoid of any charged and/or pronounced hydrophobic side chains that could form strong hydrophobic and/or electrostatic interactions. In addition, the random coil behaviour of PA(S) (poly)peptides under physiological conditions poses a considerable entropic cost for the disorder-to-order transition upon complex formation with binding proteins such as antibodies. This is also evidenced by numerous in vivo imaging studies with PASylated antibody fragments as well as in preclinical animal experiments involving repeated protein dosing, wherein neither any unspecific binding to non-target tissues or organs nor a PAS-specific immune response were detectable (Bolze et al., 2016; Harari et al., 2014; Mendler et al., 2015; Richter et al., 2020). Taken together, these PAS-specific sequence/structure characteristics therefore pose a unique and significant challenge to generate PA(S) specific antibodies, which was overcome by the inventive methods and peptides/antigens as disclosed herein. Corresponding illustrative P/A peptides/antigens are also described herein below.

The present invention relates in a particular embodiment to a method for generating specifically binding moieties, in particular antigen-binding molecules, directed against intrinsically disordered proteins and/or intrinsically disordered protein domains or peptides, said method comprising the step of immunizing a non-human mammal with an antigen whereby said antigen is a conjugate of an immunoadjuvant and one or more P/A peptides,
wherein each P/A peptide is independently a peptide R^{N}—(P/A)-R^{C},
wherein (P/A) is an amino acid sequence consisting of about 5 to about 100 amino acid residues, wherein at least 60% of the number of amino acid residues in (P/A) are independently selected from proline and alanine, wherein (P/A) includes at least one proline residue and at least one alanine residue,
wherein R^{N} is a protecting group which is attached to the N-terminal amino group of (P/A),
wherein R^{C} is an amino acid residue which is bound via its amino group to the C-terminal carboxy group of (P/A) and which comprises at least one, at least two, at least three, at least four, at least five, at least 6 carbon atom between its amino group and its carboxy group, and wherein each P/A peptide is conjugated to the immunoadjuvant via an amide linkage formed from the carboxy group of the C-terminal amino acid residue R^{C} of the P/A peptide and a free amino group of the immunoadjuvant.

In a preferred embodiment the a method for the generation of said antigen binding molecule, preferably said antibody or said antigen-binding fragment thereof (directed against intrinsically disordered peptides/proteins and/or intrinsically disordered peptide/protein domains) as described herein is a method of immunization of a non-human with said P/A peptide(s)
wherein the P/A peptide is independently a peptide R^{N}—(P/A)-R^{C} and is a peptide consisting of about 8 to about 90 amino acid residues,
wherein at least 70% of the number of amino acid residues in (P/A) are independently selected from proline and alanine, wherein (P/A) includes at least one proline residue and at least one alanine residue,
wherein R^{N} is a protecting group which is attached to the N-terminal amino group of (P/A),
wherein R^{C} is an amino acid residue which is bound via its amino group to the C-terminal carboxy group of (P/A) and which comprises at least one carbon atom between its amino group and its carboxy group, and
wherein each P/A peptide is conjugated to the immunoadjuvant via an amide linkage formed from the carboxy group of the C-terminal amino acid residue R^{C} of the P/A peptide and a free amino group of the immunoadjuvant.

As used herein, the term “specifically binding moiety/moieties” comprises in particular the herein discussed described antigen-binding molecules, antibodies and antigen-binding fragments thereof. However, the term also comprises other molecules that are able to specifically bind said intrinsically disordered peptides/proteins but are not in the common antibody format. Such “binding moieties” may, inter alia, comprise molecules like fusion proteins or (protein) constructs comprising a binding part that is based on or derived form an antibody obtainable with the means and methods provided herein. Such a construct may, inter alia, comprise at least one, at least two or three complementarity-determining regions (CDRs) of antibodies/antibody fragments provided herein and/or obtainable with the methods of this invention.

In a specific embodiment, the present invention provides for means and methods for the generation of antigen binding molecules, preferably antibodies or antigen-binding fragments thereof, directed against intrinsically disordered peptides/proteins and/or intrinsically disordered peptide/protein domains. In a preferred embodiment, the present invention provides for means and methods for the generation of antibodies and/or antigen-binding fragments thereof that are directed against and/or specifically bind to intrinsically disordered peptides/proteins (IDPs) and/or intrinsically disordered protein domains, in particular to Pro/Ala-rich sequence (“PAS”), “PAS” sequences/“PAS” moieties.

Pro/Ala-rich sequence (“PAS”), “PAS” sequences/“PAS” moieties are defined herein and are also described in WO 2008/155134 and WO 2011/144756. These “PAS” moieties, as furthermore described in (Schlapschy et al., 2013) or (Binder & Skerra, 2017), also relate to peptides consisting of at least 7 amino acid residues forming random coil conformation whereby said amino acid residues forming said random coil conformation are selected from Pro (P), Ala (A) and Ser (S) or from Pro (P) and Ala (A). The Pro/Ala-rich sequences as comprised, inter alia, in proteinaceous drug conjugates are also described as “(P/A)” sequences, for example in WO 2018/234455. These (P/A) sequences, i.e. here the Pro/Ala-rich sequences, may consist of about 7 to about 1200 amino acid residues, wherein at least 80% of the number of amino acid residues in (P/A) are independently selected from proline and alanine, wherein (P/A) includes at least one proline residue and at least one alanine residue. Yet, as is evident from the disclosure herein and also known in the art, the term “Pro/Ala-rich sequence (PAS)”, “PAS”, “PAS moiety” or “PAS sequence” is not to be construed limiting to intrinsically disordered proteins/peptides (IDPs) and/or proteins/peptides that form random coil conformation comprising Pro and Ala only. The term also encompasses corresponding proteins/peptides that are comprised mainly from Pro, Ala and Ser. Also, to a minor extend further amino acids may be comprised, as also disclosed, inter alia, in WO 2008/155134, WO 2011/144756 or WO 2018/234455 recited above (all incorporated by reference).

As discussed herein, the conjugation of drugs with such (P/A) sequences and/or Pro/Ala-rich sequences (PAS) is also known as PASylation®, which dramatically expands the hydrodynamic volume and prolongs the plasma half-life by retarding renal filtration in vivo (Griffiths et al., 2019; Langin et al., 2018; Richter et al., 2020).

Accordingly, the present invention provides means and methods for obtaining specific binding moieties, in particular antibodies and/or antigen binding fragments thereof, that specifically bind to structurally disordered and/or intrinsically disordered sequences, in particular to Pro/Ala-rich sequences (PAS). The prior art does not provide for, nor does it describe any antibodies and/or antigen binding fragments directed against structurally disordered and/or intrinsically disordered sequences, in particular Pro/Ala-rich sequences. Furthermore, it was previously described that recombinant polypeptides that are composed of Pro, Ala and Ser, or even of Pro and Ala only, are highly hydrophilic and structurally disordered, regardless of their precise amino acid sequence—if certain repeat patterns or long homo-amino acid stretches are avoided (Breibeck & Skerra, 2018; Schlapschy et al., 2013). Notably, PAS polypeptides whose amino acid sequences are precisely defined at the genetic level, are fully neutral while their side chains—in particular for the Ser-free P/A sequences—lack pronounced polar groups. Thus, the strong hydrophilicity of Pro/Ala-rich sequences (PAS) is explained by the exposure of the peptide groups to the aqueous solvent in the absence of rigid secondary structures (Breibeck & Skerra, 2018). Last but not least, due the lack of pronounced polar groups Pro/Ala-rich sequences/(PAS) sequences lack immunogenicity in animals, in particular in mammals. This low immunogenicity is indeed one of the hallmarks the PASylation® technology in the pharmaceutical and medical field.

PAS polypeptides as employed in the PASylation® technology are known to lack immunogenicity (are “immunologically inert”) in mammals, in particularly in rodents like mice, rats or rabbits, i.e. animals routinely used for the preparation of (monoclonal) antibodies. In fact, as a consequence of their restricted amino acid composition, PAS polypeptides are devoid of both charged and bulky hydrophobic side chains, which normally play a role for molecular recognition, especially in the immune response. In addition, their random coil behavior under physiological conditions poses a huge entropic cost for the disorder-to-order transition upon complex formation with binding proteins such as antibodies. This is also evidenced by numerous in vivo imaging studies with PASylated antibody fragments as well as in preclinical animal experiments involving repeated protein dosing, wherein neither any unspecific binding to non-target tissues or organs nor a PAS-specific immune response were detectable (Bolze et al., 2016; Griffiths et al., 2019; Harari et al., 2014; Mendler et al., 2015; Richter et al., 2020).

Yet, despite this lack of immunogenicity of Pro/Ala-rich sequences (PAS) in previous animal studies the present inventors have succeeded in generating binding moieties specifically binding to different Pro/Ala-rich sequences (PAS), in particular (monoclonal) antibodies. This success is based on the novel and inventive provision of specific antigens to be employed in the immunization of the non-human animals. Said inventive antigens are denoted herein comprising (P/A) sequences [(P/A) is an amino acid sequence] or (P/A) antigens [in the format R^{N}—(P/A)-R^{C} as defined herein], which are characterized by their conjugation to highly immunogenic carrier proteins (“immunoadjuvants”), like e.g. keyhole limpet hemocyanin (KLH). The (P/A) sequences or (P/A) antigens are conjugated to said immunoadjuvant via an amide linkage formed between the carboxy group of the C-terminal amino acid or linker residue (herein “R^{C}”) of the (P/A) sequence/antigen and one or more free amino group(s) of the immunoadjuvant. Furthermore, the (P/A) sequences/antigens to be employed in context of this invention are N-terminally blocked, namely by a protecting group which is attached to the N-terminal amino group of said (P/A) sequences/antigens and is denoted herein as “R^{N}”. This also obviates the formation of N-terminus specific antibodies.

The inventive method of the present invention comprises the immunization of a non-human mammal (in particular a mouse or mice) with (P/A) peptides/sequences/antigens as disclosed herein and as in particular provided in the herein discussed R^{N}—(P/A)-R^{C} form. These (P/A) sequences/antigens are used directly as immunogens as defined herein, i.e. comprising the protecting group “R^{N}” at the N-terminus and the immunoadjuvant linked to the C-terminus. However, it is also envisaged that the antigen to be used for immunization comprises a plurality of said (P/A) peptides/sequences/antigens. Accordingly, the antigen to be used in the context of this invention is a conjugate of an immunoadjuvant and one or more (P/A) peptides/sequences/antigens as disclosed herein.

Preferred (P/A) peptides/sequences/antigens may comprise:
amino acid sequences/antigens consisting of about 5 to about 100 amino acid residues, more preferably about 8 to about 90 amino acid residues, wherein at least 60%, at least 70% of the number of amino acid residues in (P/A) are independently selected from proline and alanine, wherein (P/A) includes at least one proline residue and at least one alanine residue;
amino acid sequences/antigens consisting of about 5 to about 100 amino acid residues, preferably about 10 to about 80 amino acid residues, wherein at least 70% of the number of amino acid residues in (P/A) are independently selected from proline and alanine, wherein at least 95% of the number of amino acid residues in (P/A) are independently selected from proline, alanine and serine, and wherein (P/A) includes at least one proline residue and at least one alanine residue; and/or
amino acid sequences/antigens consisting of about 20 to about 40 amino acid residues independently selected from proline, alanine and serine, wherein at least 70% of the number of amino acid residues in (P/A) are independently selected from proline and alanine, and wherein (P/A) includes at least one proline residue and at least one alanine residue.

In one embodiment of the present invention, the (P/A) peptides/sequences/antigens to be used in the methods provided herein for immunization of the non-human mammal may be (P/A) sequences/antigens wherein the proportion of the number of proline residues comprised in said (P/A) to the total number of amino acid residues comprised in (P/A) is ≥about 10% and ≤about 70%, preferably ≥2 about 20% and ≤about 50%, more preferably ≥about 25% and about ≤40%.

In accordance with the above, the (P/A) peptides/sequences/antigens to be employed in context of the present invention may be (P/A) peptides/sequences/antigens that consist of (i) five or more partial sequences independently selected from “ASPA”, “APAP”, “SAPA”, “AAPA” and “APSA”, and (ii) optionally one, two or three further amino acid residues independently selected from proline (P), alanine (A) and serine (S). The (P/A) peptides/sequences/antigens may also comprise multimers as well as combinations of these partial sequences independently selected from “ASPA”, “APAP”, “SAPA”, “AAPA” and “APSA”. In one embodiment said (P/A) peptide/sequence/antigen consists of (i) the sequence ASPA-APAP-ASPA-APAP-SAPA (SEQ ID NO: 1), (ii) the sequence AAPA-APAP-AAPA-APAP-AAPA (SEQ ID NO: 2), (iii) the sequence APSA-APSA-APSA-APSA-APSA (SEQ ID NO: 3), (iv) a duplication of any of the aforementioned sequences, or (v) a combination of at least two of the aforementioned sequences.

Non-limiting examples of such peptides/sequences/antigens are (P/A)s that consist of (i) the sequence ASPA-APAP-ASPA-APAP-SAPA-ASPA-APAP-ASPA-APAP-SAPA, (ii) the sequence AAPA-APAP-AAPA-APAP-AAPA-AAPA-APAP-AAPA-APAP-AAPA, or (iii) the sequence APSA-APSA-APSA-APSA-APSA-APSA-APSA-APSA-APSA-APSA. Also multimers of these sequences are comprised in the gist of this invention and may be employed in the immunization method of a non-human animal provided herein. Non-limiting examples of the embodiments are also provided in the experimental part as “PAS #1”, “P/A #1” or “APSA”. In the experimental part such 20mer peptides (or multimers thereof, like 40mer, as illustrated in SEQ ID Nos.: 5, 6 or 7) conjugated to the (“immunoadjuvants”) and N-terminally blocked were employed as illustrative examples. Surprisingly, several monoclonal antibodies (MAbs) with high binding activity and specificity towards PAS sequence motifs were obtained with the novel and inventive method disclosed herein.

Therefore, and in a preferred embodiment of the invention, the method for the generation of said antigen binding molecule, said antibody and/or said antigen-binding fragment comprises immunization of a non-human animal with an antigen that comprises one or more P/A peptide(s)
wherein said P/A peptide is independently a peptide

R^{N}—(P/A)-R^{C},
wherein said P/A peptide is a peptide consisting of about 5 to about 100 amino acid residues and wherein at least 70% of the number of amino acid residues in (P/A) are independently selected from proline and alanine, wherein (P/A) includes at least one proline residue and at least one alanine residue,
wherein R^{N} is a protecting group which is attached to the N-terminal amino group of (P/A),
wherein R^{C} is an amino acid residue which is bound via its amino group to the C-terminal carboxy group of (P/A) and which comprises at least one carbon atom between its amino group and its carboxy group, and
wherein each P/A peptide is conjugated to the immunoadjuvant via an amide linkage formed from the carboxy group of the C-terminal amino acid residue R^{C} of the P/A peptide and a free amino group of the immunoadjuvant.

Also, in the context of this preferred embodiment of the present invention, the explanations for the P/A peptide/antigen and/or (P/A) provided herein above apply here.

As discussed herein above, the inventors were surprisingly successful with the herein provided means and methods in the generation of (non-human) monoclonal antibodies (MAbs) directed against intrinsically disordered proteins and/or intrinsically disordered protein domains or peptides, in particular against PAS sequences as, inter alia, employed in the known PASylation® approach/technology. These intrinsically disordered proteins and/or intrinsically disordered protein domains or peptides or PAS polypeptides as employed in the PASylation® technology are known to lack immunogenicity (are “immunologically inert”) in mammals, in particular in rodents like mice, rats or rabbits, i.e. animals routinely used for the preparation of (monoclonal) antibodies. This success, as illustrated herein and in the appended experimental part, in the examples and in the appended figures, is a result of the use of antigens for immunization that consist of and/or that comprise the herein defined P/A peptides/antigens or (P/A)s (or multimers thereof). As discussed herein, said P/A peptide/antigen may adopt a random coil conformation. Furthermore, the antigen may comprise two or more “P/A peptides” as defined herein. The P/A peptides comprised in said antigen may be multiple copies of the same P/A sequences as defined herein, like, non-limiting, sequences independently selected from “ASPA”, “APAP”, “SAPA”, “AAPA” and “APSA”. Examples are provided herein and are also illustrated in sequences like SEQ ID NOs.: 5, 6 or 7. Again the antigens to be employed in the context of the present invention are antigen conjugates of an immunoadjuvant and one or more P/A peptides, wherein ach P/A peptide is independently a peptide of the structure

R^{N}—(P/A)-R^{C}.

In context of this invention, “R^{N}” is a protecting group which is attached to the N-terminal amino group of the herein defined (P/A) amino acid sequence. Said “R^{N}” may be selected from pyroglutamoyl (Pga; known as 2-pyrrolidone-5-carboxylic acid or 5-oxoproline), homopyroglutamoyl, formyl, acetyl, hydroxyacetyl, methoxyacetyl, ethoxyacetyl, propoxyacetyl, propionyl, 2-hydroxypropionyl, 3-hydroxypropionyl, 2-methoxypropionyl, 3-methoxypropionyl, 2-ethoxypropionyl, 3-ethoxypropionyl, butyryl, 2-hydroxybutyryl, 3-hydroxybutyryl, 4-hydroxybutyryl, 2-methoxybutyryl, 3-methoxybutyryl, 4-methoxybutyryl, glycine betainyl, o-aminobenzoyl, —NH—(C₁₋₆ alkyl), —N,N(C₁₋₈ alkyl)₂, N,N,N-tri(C₁₋₆ alkyl)₃, N,N-tetramethylene, and N,N-pentamethylene.

It will be understood that if R^{N} is a group N—(C₁₋₆ alkyl), N,N-di(C₁₋₆ alkyl) or N,N,N-tri(C₁-s alkyl), there will be one, two or three C₁₋₆ alkyl groups bound to the nitrogen atom of the amino group of the (P/A) moiety to be protected. In the case of two or three alkyl groups bound to the nitrogen atom, the respective alkyl groups are each independently a C₁₋₆ alkyl group and may thus be the same or different. In the case of three alkyl groups said nitrogen atom will be an ammonium group.

Moreover, if R^{N} is a group N,N-tetramethylene or N,N-pentamethylene, it will be understood that both ends of the tetramethylene or pentamethylene carbon chain will be attached to the nitrogen atom of the same amino group to be protected, and will thus form a saturated 5- or 6-membered ring (i.e., a pyrrolidine or piperidine ring) together with the nitrogen atom they are attached to.

As used herein, “R^{C}” is an amino acid residue which is bound via its amino group to the C-terminal carboxy group of the herein defined (P/A) amino acid sequence and it comprises at least one, at least two, at least three, at least four, at least five or six carbon atom between its amino group and its carboxy group. In a preferred embodiment, said “R^{C}” may be H₂N—(C₁₋₁₂ hydrocarbyl). —COOH. In another preferred embodiment, “R^{C}” may be selected from the group consisting of H₂N—(CH₂)₁₋₁₀—COOH, H₂N-phenyl-COOH, and H₂N-cyclohexyl-COOH. Even more preferred is that “R^{C}” is selected from the group consisting of H₂N—CH₂—COOH (Gly), H₂N—(CH₂)₂—COOH (β-Ala), H₂N—(CH₂)₃—COOH, H₂N—(CH₂)₄—COOH, H₂N—(CH₂)₅—COOH, H₂N—(CH₂)₆—COOH, H₂N—(CH₂)₇—COOH, H₂N—(CH₂)₈—COOH, p-aminobenzoic acid, and 4-aminocyclohexanecarboxylic acid. Most preferred is that “R^{C}” is H₂N—(CH₂)₅—COOH (aminohexanoic acid).

In a preferred embodiment and is illustrated in the appended examples, the P/A peptide(s) comprised in the antigen to be employed in the means and methods of the present invention adopt(s) a random coil conformation. Furthermore, said P/A peptide(s) comprised in said antigen is/are devoid of charged residues. As discussed herein, also, to a minor extend further amino acids may be comprised, as also disclosed, inter alia, in WO 2008/155134, WO 2011/144756 or WO 2018/234455 recited above (all incorporated by reference). Also, these further amino acids are preferably devoid of any charged residues and/or devoid of any pronounced hydrophobic side chains. An exemplary, non-limiting amino acid may be glycine.

The antigen to be employed in the context of the present invention and as provided herein is a conjugate of an immunoadjuvant and one or more P/A peptides as defined herein. Such “immunoadjuvants” are known in the art and are described as highly immunogenic carrier proteins which are not exclusively but preferably foreign (i.e. derived from a different species) to the subject that these highly immunogenic carrier proteins are to be administered to. Preferably, and as illustrated in the examples, such immunoadjuvants form protein complexes with a molecular mass of more than about 5 megadaltons (5 000 000 Da). A preferred example of such immunoadjuvants is KLH. Likewise, these highly immunogenic carrier proteins induce detectable antibody titers which are directed (i.e. antibodies that bind) against (to) the “immunoadjuvants”/highly immunogenic carrier proteins themselves. In the context of this invention, the P/A peptide or (P/A) amino acid sequence defined herein is conjugated to an ε-amino group of a lysine residue or a free N-terminal amino group of said immunoadjuvant. The immunoadjuvant may be, without being limiting, selected from the group consisting of keyhole limpet hemocyanin (KLH), ovalbumin (OVA), and bovine serum albumin (BSA). Preferably, said immunoadjuvant is keyhole limpet hemocyanin (KLH).

In accordance with the above, non-limiting examples of the antigen of the present invention and to be employed in the context of the means and methods provided herein are:

Pga-PAS#1(40)-Ahx:
(Pga-ASPA-APAP-ASPA-APAP-SAPA-ASPA-APAP-ASPA-APAP-
SAPA-Ahx; SEQ ID NO: 5);
Pga-P/A#1(40)-Ahx:
(Pga-AAPA-APAP-AAPA-APAP-AAPA-AAPA-APAP-AAPA-APAP-
AAPA-Ahx; SEQ ID NO: 6);
Pga-APSA(40)-Ahx:
(Pga-APSA-APSA-APSA-APSA-APSA-APSA-APSA-APSA-APSA-
APSA-Ahx; SEQ ID NO: 7).

Pga means a pyroglutamyl residue (also known as 2-pyrrolidone-5-carboxylic acid or 5-oxoproline) and Ahx means aminohexanoic acid. Accordingly, these illustrative 40mer “P/A” peptides are designed to encompass at least two copies of a correspond “PAS sequence repeat”. The peptides contain chemically inert side chains only and have a blocked N-terminus, their single C-terminal carboxylate group (in fact, the one of the Ahx linker residue) is activated selectively and is used for directed chemical conjugation to the ε-amino groups of Lys side chains of immunoadjuvant, i.e, in the appended example, KLH.

Therefore, the present invention provides, in one embodiment, novel and inventive antigens which can be, inter alia, employed without further ado in the inventive methods for generating antigen-binding molecules (in particular antibodies) directed against intrinsically disordered proteins and/or intrinsically disordered protein domains or peptides in non-human animals, in particular in rodents, like mice and rats, but also in other mammals, comprising and non-limiting horse, sheep, goats, camelids, etc. Accordingly, the present invention also relates to the antigen(s) as defined and provided herein. Also, a gist of the present invention is the use of this/these antigen(s) in the method of the present invention. Therefore, the present invention also relates to the non-therapeutic use of the antigen as provided herein for the generation of an antigen binding molecule, preferably an antibody or an antigen-binding fragment thereof, directed against intrinsically disordered peptides/proteins and/or intrinsically disordered peptide/protein domains, whereby said use comprises the immunization of a non-human mammal.

The binding moieties, in particular the antigen-binding molecules, most particularly the antibodies or the antigen-binding fragment thereof, as obtainable and obtained by the present invention are directed against intrinsically disordered proteins and/or intrinsically disordered protein domains or peptides. These binding moieties, antigen-binding molecules, antibodies or the antigen-binding fragment thereof are also part of this invention and they bind, preferably and specifically, to structurally disordered and/or intrinsically disordered sequences, in particular to Pro/Ala-rich sequences (PAS), as also known in the art. Such Pro/Ala-rich sequence (PAS) are defined herein and are also described in WO 2008/155134 and WO 2011/144756. As discussed above, these “PAS” moieties, as furthermore described in (Schlapschy et al., 2013) or (Binder & Skerra, 2017), also relate to peptides consisting of at least 7 amino acid residues and to about 2000 amino acid residues forming random coil conformation whereby said amino acid residues forming said random coil conformation are selected from Pro (P), Ala (A) and Ser (S) or from Pro (P) and Ala (A). The “binding targets” of the herein provided antigen-binding molecules, most particularly of the antibodies or an antigen-binding fragment thereof, are therefore, in a preferred embodiment, intrinsically disordered proteins and/or intrinsically disordered protein domains which are Pro/Ala-rich sequences (PAS) and/or which are amino acid sequences consisting of at least 10, at least 20, at least 40, at least 50, at least 60, at least 80, at least 100, at least 120, at least 140, at least 160, at least 180, at least 190, at least 200, or about 200, about 250, about 300, about 350, about 400, about 450, about 500, about 550, about 600, about 650, about 700, about 750, about 800, about 850, about 900, about 950 about 1000, about 1500 or about 2000 amino acid residues forming random coil conformation and whereby said amino acid residues forming said random coil conformation are selected from Pro (P), Ala (A) and Ser (S) or are Pro (P) and Ala (A). Further definitions and explanations of Pro/Ala-rich sequences (PAS) that form random coil conformation are, inter alia, provided in WO 2008/155134 and WO 2011/144756, both of which are herewith incorporated by reference.

In further embodiments of the present invention, the binding moieties, in particular the antigen-binding molecules, most particularly the antibodies or antigen-binding fragments thereof may bind to Pro/Ala-rich sequences (PAS molecule; “PAS”), wherein said PAS may be an amino acid sequence consisting of about 7 to about 2000, preferably about 7 to about 1200 amino acid residues, wherein at least 80% of the number of amino acid residues in “PAS” are independently selected from proline and alanine and wherein said (PAS) includes at least one proline residue and at least one alanine residue. Said “PAS” may also be an amino acid sequence consisting of about 8 to about 400 amino acid residues, wherein at least 85% of the number of amino acid residues in “PAS” are independently selected from proline and alanine, and wherein at least 95% of the number of amino acid residues in “PAS” are independently selected from proline, alanine, glycine and serine, and wherein “PAS” includes at least one proline residue and at least one alanine residue. The inventive binding moieties may also specifically bind to Pro/Ala-rich sequences (PAS molecule; “PAS”), wherein “PAS” is an amino acid sequence consisting of 10 to 60 amino acid residues independently selected from proline, alanine, glycine and serine, wherein at least 95% of the number of amino acid residues in “PAS” are independently selected from proline and alanine, and wherein “PAS”) includes at least one proline residue and at least one alanine residue. Corresponding “PAS molecules are also described in WO 2018/234455, which is also incorporated by reference.

Accordingly, and in a particular embodiment, the inventive binding moieties, antigen-binding molecules or antibodies (or antigen-binding fragment thereof] specifically bind to Pro/Ala-rich sequences (PAS) and/or to amino acid sequences consisting of at least 20, preferably at least 40, preferably at least 60, preferably of at least 80, more preferably of at least 100, more preferably at least 120, more preferably at least 140, more preferably at least 160, more preferably at least 180, more preferably at least 200, more preferably, more preferably at least 300 to about 1200 amino acid residues forming random coil conformation and whereby said amino acid residues forming said random coil conformation are selected from Pro (P), Ala (A) and Ser (S) or are Pro (P) and Ala (A). Therefore, the preferred target Pro/Ala-rich sequences (PAS molecule; “PAS”) of the inventive binding moieties comprise or consist of alanine, serine and proline or comprise alanine and proline.

In one embodiment of the present invention, the inventive binding moieties, in particular the antigen-binding molecules, most particularly the antibodies or antigen-binding fragments thereof, may bind to and/or detect at least one epitope on said PAS target sequence. This epitope may be a linear epitope, but it may also be an epitope provided by three dimensional structure(s). The appended, non-limiting examples provide ample evidence for corresponding binding studies, including epitope mappings, SPOT epitope analyses, antigen affinity measurements (e.g. by ELISAs), surface plasmon resonance (SPR) real-time measurements, Western blotting, but also by co-crystallization of antigen-binding fragments (in particular Fab fragments) etc. Without being limiting, and in one embodiment of the present invention, the inventive antigen-binding molecules, most particularly the antibodies or antigen-binding fragments thereof, may bind Pro/Ala-rich sequences that comprise at least one epitope of the structure
(P/S)A(A/S)P and/or
PA(A/S)P.

Said epitope may be or may comprise an epitope stretch selected from the group consisting of PAPAAP (SEQ ID NO: 8), PAPASP (SEQ ID NO: 9), PASPAAP (SEQ ID NO: 10), PSAAPS (SEQ ID NO: 79), ASPAAP (SEQ ID NO: 80), PASPAA (SEQ ID NO: 81), PAAP (SEQ ID NO: 82), PASP (SEQ ID NO: 83), APSA (SEQ ID NO: 84) and PSAA (SEQ ID NO: 85).

As is illustrated herein in the appended examples and herein below, the present invention provides a plurality of novel and inventive antibodies or antigen-binding fragments thereof.

Again, without being limiting and without being bound by theory, an epitope detection of “PAAP” is deduced for anti-PA(S) MAb 2.2, anti-PA(S) MAb 2.1, anti-PA(S) MAb 1.1 and anti-PA(S) MAb 1.2;
an epitope detection of “PASP” is deduced for anti-PA(S) MAb 2.2, anti-PA(S) MAb 2.1, anti-PA(S) MAb 1.1 and anti-PA(S) MAb 1.2;
an epitope detection “PAPASP” is deduced for anti-PA(S) MAb 2.2 and anti-PA(S) MAb 2.1;
an epitope detection “PAPAAP” is deduced for anti-PA(S) MAb 2.2 and anti-PA(S) MAb 2.1;
an epitope detection “PASPAAP” is deduced for anti-PA(S) MAb 1.1 and anti-PA(S) MAb 1.2;
an epitope detection “PASPAA” is deduced for anti-PA(S) MAb 1.1;
an epitope detection “ASPAAP” is deduced for anti-PA(S) MAb 1.1 and anti-PA(S) MAb 1.2;
an epitope detection “APSA” is deduced for anti-PA(S) MAb 3.1 and anti-PA(S) MAb 3.2;
an epitope detection “PSAA” is deduced for anti-PA(S) MAb 3.1 and anti-PA(S) MAb 3.2;
an epitope detection “PSAAPS” is deduced for anti-PA(S) MAb 3.2.

As also the appended, non-limiting but highly illustrative (co-)crystallization data of the antigen-binding molecules (Fab fragments) provided herein, further epitope studies of the examples illustrate that the inventive antigen-binding molecules (i.e. antibodies/antigen-binding fragments thereof) bind to epitopes comprising alanine residues (A, Ala). Interestingly, at least one Ala residue of the Pro/Ala-rich sequences is involved in relevant interactions with the anti-PAS Fab; Therefore, and without being limiting, alanine may be considered as a “hot spot” for interactions of the inventive antibodies with PAS epitopes within Pro/Ala-rich sequences. Up to the present invention, Ala, the amino acid with the smallest side chain, has been regarded to play a negligible role in protein-protein/peptide recognition. In fact, the strategy of alanine-scanning mutagenesis (Cunningham & Wells, 1989) has found wide application to dissect critical residues for receptor-ligand or antibody-antigen binding, assuming a quasi inert role of the Ala methyl side chain for molecular interactions. Unexpectedly, this invention reveals that Ala actually can adopt a central role in antigen recognition, as exemplified in particular by the crystal structure studies provided in context of this invention.

In this context, the present invention also provides a complex between the inventive binding moieties, antigen-binding molecules, antibodies/antigen-binding fragments thereof, and a Pro/Ala-rich sequence (PAS) molecule and an epitope as provided herein, in particular an alanine-comprising epitope. These epitopes may comprise structures like (P/S)A(A/S)P and/or PA(A/S)P. Examples are provided herein.

In a further embodiment of the invention, complexes are provided and claimed herein between the specifically binding moiety as obtainable by the means and methods provided herein, in particular the antigen-binding molecules (like antibodies and antigen-binding fragments thereof) of the invention and a Pro/Ala-rich sequence/(PAS) molecule. Also, complexes between the binding moiety or the antigen-binding molecules (like antibodies and antigen-binding fragments thereof) of the invention and fusion proteins and/or drug conjugates comprising a Pro/Ala-rich sequence ((PAS) molecule are part of this invention. Such “anti-‘PAS’ complexes” of the present invention are in particular useful, without being limiting, in the methods of diagnosis, screenings but also as research tools provided herein.

As discussed above and as illustrated in the appended examples, the present inventors provide for the first time binding moieties, antigen-binding molecules, antibodies/antigen-binding fragments thereof that specifically bind intrinsically disordered proteins and/or intrinsically disordered protein domains or peptides, in particular, Pro/Ala-rich sequence (PAS) molecules and/or epitopes comprised by or formed by these Pro/Ala-rich sequence (PAS) molecules.

Therefore, the present invention comprises binding moieties, antigen-binding molecules, antibodies/antigen-binding fragments thereof as obtainable and/or as obtained by the means and in particular the methods provided herein. Therefore, the invention also provides a specifically binding moiety, preferably an antigen-binding molecule, more preferably an antibody obtainable by the method provided herein and/or a specifically binding moiety, preferably an antigen-binding molecule, more preferably an antibody that specifically binds to intrinsically disordered proteins and/or intrinsically disordered protein domains or to an antigenic portion of said intrinsically disordered proteins and/or intrinsically disordered protein domain,
(i) wherein said intrinsically disordered proteins and/or intrinsically disordered protein domains are Pro/Ala-rich sequences (PAS) and/or are amino acid sequences consisting of at least 20 amino acid residues forming random coil conformation and whereby said amino acid residues forming said random coil conformation are selected from Pro (P), Ala (A) and Ser (S) or are Pro (P) and Ala (A) and/or
(ii) wherein said specifically binding moiety, preferably said antigen-binding molecule binds to an epitope of the structure: (P/S)A(A/S)P and/or PA(A/S)P. Such epitopes may be selected form the group consisting of PAPAAP (SEQ ID NO: 8), PAPASP (SEQ ID NO: 9), PASPAAP (SEQ ID NO: 10), PSAAPS (SEQ ID NO: 79), ASPAAP (SEQ ID NO: 80), PASPAA (SEQ ID NO: 81), PAAP (SEQ ID NO: 82), PASP (SEQ ID NO: 83), APSA (SEQ ID NO: 84) and PSAA (SEQ ID NO: 85).

The binding moieties of the invention may be antigen-binding molecules as well as antibodies (MAbs) or antigen-binding fragments thereof (e.g. Fabs). Said antigen-binding molecule may be an immunoglobulin (Ig), an antibody, an antigen-binding fragment thereof, a bispecific antibody, an IgG antibody, a Camel/Llama heavy chain antibody (camelid antibody), an immunoglobulin novel antigen receptor (IgNAR) or an antibody mimetic. The invention also comprises antibodies, antigen-binding fragments thereof of antibody constructs that are engineered via recombinant means on the basis of the binding moieties, antigen-binding molecules as well as antibodies or antigen-binding fragments thereof of the invention and as obtainable by the means and methods provided herein. For example, the corresponding sequence information of the antigen-binding molecule may by employed in the construction of such engineered/recombinant binding moieties/antigen-binding molecules. Such an engineered/recombinant binding moiety/antigen-binding molecule may, inter alia, be based on the CDR sequences of the antibodies obtained by the method of the present invention or as illustratively provided herein.

The present invention also provides antigen-binding molecules/antibodies which may be selected form the group consisting of a monoclonal antibody, a chimeric antibody, a recombinant antibody and an antigen-binding fragment of a recombinant or chimeric antibody. An inventive antigen-binding fragment may be, without being limiting, a Fab fragment, a Fab′ fragment, a (Fab′)₂ fragment, a single chain variable fragment (scFv), a single-domain antibody or fragment such as a VHH domain or nanobody. The term “antibody” as used herein also comprises a humanized antibody or an antibody displayed on the surface of a phage, a yeast cell, a bacterial cell or a mammalian cell. The antibody of the invention may be an IgG1, IgG2, IgG2a or IgG2b, IgG3 or IgG4 antibody.

The PAS-binding moieties/antibodies (Anti-PA(S) MAbs) of the present invention show substantially no or very low cross-reactivity with proteins that lack structurally disordered PAS sequences, sequence stretches, (poly)peptide segments or protein domains. In particular, said Anti-PA(S) MAbs show no or very low cross-reactivity with human blood plasma proteins and/or plasma proteins from primates, mammals, rodents, in particular from monkeys, macaques, baboons, mice, rats, rabbits, dogs, pigs, cattle, sheep. Furthermore, and in another embodiment, said Anti-PA(S) MAbs show no or very low cross-reactivity with host cell proteins from production organisms as typically employed in the areas of recombinant protein production, genetic engineering or biotechnology, for example bacteria, like Escherichia coli, Corynebacterium glutamicum or Pseudomonas fluorescens, or yeasts, like Saccharomyces cerevisiae or Pichia pastoris, or mammalian cells, like CHO, HEK, NSO or COS cells.

The PAS-binding moieties/antibodies (Anti-PA(S) MAbs) according to the invention show high affinities/low dissociation constants (K_{D} values) toward PAS sequences, PAS polypeptides and/or PAS fusion proteins or conjugates. Such K_{D} values can be determined using many techniques well known in the art, for example using ELISAs or SPR measurements as illustrated in the examples disclosed herein further below. Of note, such measurements can be performed for the intact antibodies (MAbs) or for antigen-binding fragments thereof, for example Fab fragments, Fv or scFv fragments, and corresponding K_{D} values may vary depending on the type of antibody protein (intact or fragment) and the precise assay used (ELISA, SPR, fluorescence titration and the like). In general, preferred K_{D} values are less than 500 μM, less than 200 μM, less than 100 μM, less than 50 μM, less than 10 μM, preferably less than 1 μM, less than 500 nM, less than 200 nM, less than 100 nM, less than 50 nM, less than 20 nM, less than 10 nM, less than 5 nM, less than 2 nM and even more preferably less than 1 nM, less than 500 μM, less than 200 μM or less than 100 μM. For use of an Anti-PA(S) MAb according to the invention in bioanalytical or diagnostic assays, particularly low K_{D} values are preferred, such as less than 10 nM, less than 5 nM or less than 2 nM and even more preferably less than 1 nM, less than 500 μM, less than 200 μM or less than 100 μM.

Without being bound by theory, but as also shown in the appended examples, the apparent affinity of the inventive binding moieties/antibodies is influenced by the avidity effect and appears to be most pronounced for a bivalent MAb when interacting with a long PAS sequence repeat containing multiple epitopes. X-ray structural analysis of recombinant Fab fragments of the inventive antibodies in complex with their cognate PAS epitope peptides revealed that the interactions are dominated by hydrogen bond networks with the peptide backbone as well as multiple van der Waals interactions resulting from intimate shape complementarity. As discussed above and most surprisingly, Ala, which is the amino acid with the smallest side chain (apart from Gly, which lacks a side chain), emerged as a crucial feature for antigen recognition for the inventive binding moieties/antibodies. Said Ala provides major contributions at the center of the paratope in different “anti-PAS complexes”.

The present invention also provides specific, yet none-limiting examples of inventive binding moieties/antigen-binding molecule/antibodies and/or antigen-binding fragments of these inventive antibodies. Also in this context, the term “antigen-binding molecule” comprises an antigen-binding fragment, whereas this term in particular comprises preferably an antigen-binding fragment of the inventive antibodies provided herein and, directed against intrinsically disordered peptides/proteins and/or intrinsically disordered peptide/protein domains as described herein and/or, which is obtainable by the method of the present invention.

Accordingly, the present invention also provides antigen-binding molecule, wherein said antigen-binding molecule is selected from the group consisting of:
a) an antibody or an antigen-binding fragment thereof, comprising
   a variable heavy (VH) chain comprising
   the CDR-H1 as defined in SEQ ID NO: 35 [anti-PA(S) MAb 1.1],
   the CDR-H2 as defined in SEQ ID NO: 36 [anti-PA(S) MAb 1.1], and
   the CDR-H3 as defined in SEQ ID NO: 37 [anti-PA(S) MAb 1.1]; and/or
   a variable light (VL) chain comprising
   the CDR-L1 as defined in SEQ ID NO: 38 [anti-PA(S) MAb 1.1],
   the CDR-L2 as defined in SEQ ID NO: 39 [anti-PA(S) MAb 1.1], and
   the CDR-L3 as defined in SEQ ID NO: 40 [anti-PA(S) MAb 1.1]; or is an antibody or an antigen-binding fragment thereof binding to the same epitope as
   an antibody comprising any one or more of the CDRs of (a);
b) an antibody or an antigen-binding fragment thereof, comprising
   a variable heavy (VH) chain comprising
   the CDR-H1 as defined in SEQ ID NO: 41 [anti-PA(S) MAb 1.2],
   the CDR-H2 as defined in SEQ ID NO: 42 [anti-PA(S) MAb 1.2], and
   the CDR-H3 as defined in SEQ ID NO: 43 [anti-PA(S) MAb 1.2]; and/or
   a variable light (VL) chain comprising
   the CDR-L1 as defined in SEQ ID NO: 44 [anti-PA(S) MAb 1.2],
   the CDR-L2 as defined in SEQ ID NO: 45 [anti-PA(S) MAb 1.2], and
   the CDR-L3 as defined in SEQ ID NO: 46 [anti-PA(S) MAb 1.2]; or
   is an antibody or an antigen-binding fragment thereof binding to the same epitope as an antibody comprising any one or more of the CDRs of (b);
c) an antibody or an antigen-binding fragment thereof, comprising
   a variable heavy (VH) chain comprising
   the CDR-H1 as defined in SEQ ID NO: 47 [anti-PA(S) MAb 2.1],
   the CDR-H2 as defined in SEQ ID NO: 48 [anti-PA(S) MAb 2.1], and
   the CDR-H3 as defined in SEQ ID NO: 49 [anti-PA(S) MAb 2.1]; and/or
   a variable light (VL) chain comprising
   the CDR-L1 as defined in SEQ ID NO: 50 [anti-PA(S) MAb 2.1],
   the CDR-L2 as defined in SEQ ID NO: 51 [anti-PA(S) MAb 2.1], and
   the CDR-L3 as defined in SEQ ID NO: 52 [anti-PA(S) MAb 2.1]; or
   is an antibody or an antigen-binding fragment thereof binding to the same epitope as an antibody comprising any one or more of the CDRs of (c);
d) an antibody or an antigen-binding fragment thereof, comprising
   a variable heavy (VH) chain comprising
   the CDR-H1 as defined in SEQ ID NO: 53 [anti-PA(S) MAb 2.2],
   the CDR-H2 as defined in SEQ ID NO: 54 [anti-PA(S) MAb 2.2], and
   the CDR-H3 as defined in SEQ ID NO: 55 [anti-PA(S) MAb 2.2]; and/or
   a variable light (VL) chain comprising
   the CDR-L1 as defined in SEQ ID NO: 56 [anti-PA(S) MAb 2.2],
   the CDR-L2 as defined in SEQ ID NO: 57 [anti-PA(S) MAb 2.2], and
   the CDR-L3 as defined in SEQ ID NO: 58 [anti-PA(S) MAb 2.2]; or
   is an antibody or an antigen-binding fragment thereof binding to the same epitope as an antibody comprising any one or more of the CDRs of (d);
e) an antibody or an antigen-binding fragment thereof, comprising
   a variable heavy (VH) chain comprising
   the CDR-H1 as defined in SEQ ID NO: 59 [anti-PA(S) MAb 3.1],
   the CDR-H2 as defined in SEQ ID NO: 60 [anti-PA(S) MAb 3.1], and
   the CDR-H3 comprising or consisting of the amino acid sequence Trp-Gly-Arg; and/or
   a variable light (VL) chain comprising
   the CDR1-L as defined in SEQ ID NO: 62 [anti-PA(S) MAb 3.1],
   the CDR2-L as defined in SEQ ID NO: 63 [anti-PA(S) MAb 3.1], and
   the CDR3-L as defined in SEQ ID NO: 64 [anti-PA(S) MAb 3.1]; or
   is an antibody or an antigen-binding fragment thereof binding to the same epitope as an antibody comprising any one or more of the CDRs of (e); and
f) an antibody or an antigen-binding fragment thereof, comprising
   a variable heavy (VH) chain comprising
   the CDR-H1 as defined in SEQ ID NO: 65 [anti-PA(S) MAb 3.2],
   the CDR-H2 as defined in SEQ ID NO: 66 [anti-PA(S) MAb 3.2], and
   the CDR-H3 as defined in SEQ ID NO: 67 [anti-PA(S) MAb 3.2]; and/or
   a variable light (VL) chain comprising
   the CDR-L1 as defined in SEQ ID NO: 68 [anti-PA(S) MAb 3.2],
   the CDR-L2 as defined in SEQ ID NO: 69 [anti-PA(S) MAb 3.2], and
   the CDR-L3 as defined in SEQ ID NO: 70 [anti-PA(S) MAb 3.2]; or
   is an antibody or an antigen-binding fragment thereof binding to the same epitope as an antibody comprising any one or more of the CDRs of (f)

The sequence “Trp-Gly-Arg” as comprising anti-PA(S) Mab 3.1 is indicated as SEQ ID NO: 61 herein. Yet, it is to be understood that in the appended sequence listing this SEQ ID is represented as “000” as the sequence only consists of 3 amino acids and BiSSAP does not allow to include sequences with only 3 amino acid residues. Also, the ST.25 standard indicates that only sequences with a length of 4 and more amino acid residues shall be included in a corresponding sequence listing.

In one embodiment, the present invention relates to an antigen-binding molecule that binds to the same epitope as any of the antibodies or antigen-binding fragments of the present invention or as obtainable by the means and methods of the present invention. In one particular embodiment of this invention, said antigen-binding molecule binds to the same epitope as any of the antibodies or antigen-binding fragments defined herein above under (a) to (f).

As discussed herein above, the present invention also comprises antigen-binding molecules that are antigen-binding fragments of the inventive antibodies. These antigen-binding fragments may be selected from the group consisting of a Fab fragment, a F(ab′)₂ fragment, a Fv fragment or a scFv fragment. Such antigen-binding fragments have been illustrated in the appended examples including even data from protein crystallography and on epitope binding. Also other means and methods for the elucidation of epitopes and well as for epitope binding are amply provided in the appended experimental part. Corresponding techniques comprise immunological assays, like ELISAs and Western blots, as well as SPOT assays for epitope mapping, and also more elaborate techniques like X-ray structural analysis of e.g. complexes between recombinant Fab fragments and PAS epitope peptides. Yet, the person skilled in the art is readily in a position to deduce the epitope binding of a given antigen-binding molecule, including an antibody and/or an antigen-binding fragment thereof.

In the context of this invention, the term “binding to the same epitope” is not limited to linear epitopes but it may also comprise the binding to the same three-dimensional conformation or to a “conformational” epitope.

In a further embodiment, the present invention relates to an antigen-binding molecule, in particular an antibody or an antigen-binding fragment thereof, wherein said antigen-binding molecule, antibody or antigen-binding fragment thereof
a) comprises a variable heavy (VH) chain sequence comprising the amino acid sequence of SEQ ID NO: 11 [anti-PA(S) MAb 1.1], SEQ ID NO: 13 [anti-PA(S) MAb 1.2], SEQ ID NO: 15 [anti-PA(S) MAb 2.1], SEQ ID NO: 17 [anti-PA(S) MAb 2.2], SEQ ID NO: 19 [anti-PA(S) MAb 3.1] or SEQ ID NO: 21 [anti-PA(S) MAb 3.2]
   or a sequence having 85%, preferably 87%, more preferably at least 90% sequence identity to SEQ ID NO: 11, 13, 15, 17, 19 or 21; and
   a variable light (VL) chain sequence comprising the amino acid sequence of SEQ ID NO: 12 [anti-PA(S) MAb 1.1], SEQ ID NO: 14 [anti-PA(S) MAb 1.2], SEQ ID NO: 16 [anti-PA(S) MAb 2.1], SEQ ID NO: 18 [anti-PA(S) MAb 2.2], SEQ ID NO: 20 [anti-PA(S) MAb 3.1] or SEQ ID NO: 22 [anti-PA(S) MAb 3.2] or a sequence having 85%, preferably 87%, more preferably at least 90% sequence identity to SEQ ID NO: 12, 14, 16, 18, 20 or 22; or
b) is an antibody binding to the same epitope as an antibody of (a).

In this context, SEQ ID NOs: 11, 13, 15, 17, 19 or 21 provide heavy chain variable regions/variable heavy (VH) chain sequences of illustrative antibodies whereas SEQ ID NOs: 12, 14, 16, 18, 20 or 22 provide light chain variable regions/light heavy (VL) chain sequences of illustrative antibodies. Of note, CDR-H3 of the heavy chain SEQ ID NO: 19 [anti-PA(S) MAb 3.1] as shown in SEQ ID NO: 19 is relatively short and comprises merely 3 amino acids, namely the amino acids Trp-Gly-Arg (SEQ ID NO: 61; characterized by the “000” sequence as place holder in the appended sequence protocol for “anti-PA(S) MAb 3.1”.

A particularly preferred antigen-binding molecule or antibody of the invention, namely the antibody denoted herein as anti-PA(S) MAb 1.1, is an antigen-binding molecule or antibody that
a) comprises a variable heavy (VH) chain comprising CDR-H1 as defined in SEQ ID NO: 35, CDR-H2 as defined in SEQ ID NO: 36 and CDR-H3 as defined in SEQ ID NO: 37 and a variable light (VL) chain sequence comprising CDR-L1 as defined in SEQ ID NO: 38, CDR-L2 as defined in SEQ ID NO: 39 and CDR-L3 as defined in SEQ ID NO: 40; or
b) is an antibody binding to the same epitope as an antibody of (a).

A further preferred antigen-binding molecule or antibody of the invention, namely the antibody denoted herein as anti-PA(S) MAb 1.2, is an antigen-binding molecule or antibody that
a) comprises a variable heavy (VH) chain comprising CDR-H1 as defined in SEQ ID NO: 41, CDR-H2 as defined in SEQ ID NO: 42 and CDR-H3 as defined in SEQ ID NO: 43 and a variable light (VL) chain sequence comprising CDR-L1 as defined in SEQ ID NO: 44, CDR-L2 as defined in SEQ ID NO: 45 and CDR-L3 as defined in SEQ ID NO: 46; or
b) is an antibody binding to the same epitope as an antibody of (a).

A further preferred antigen-binding molecule or antibody of the invention, namely the antibody denoted herein as anti-PA(S) MAb 2.1, is an antigen-binding molecule or antibody that
a) comprises a variable heavy (VH) chain comprising CDR-H1 as defined in SEQ ID NO: 47, CDR-H2 as defined in SEQ ID NO: 48 and CDR-H3 as defined in SEQ ID NO: 49 and a variable light (VL) chain sequence comprising CDR-L1 as defined in SEQ ID NO: 50, CDR-L2 as defined in SEQ ID NO: 51 and CDR-L3 as defined in SEQ ID NO: 52; or
b) is an antibody binding to the same epitope as an antibody of (a).

A further preferred antigen-binding molecule or antibody of the invention, namely the antibody denoted herein as anti-PA(S) MAb 3.1, is an antigen-binding molecule or antibody that
a) comprises a variable heavy (VH) chain comprising CDR-H1 as defined in SEQ ID NO: 59, CDR-H2 as defined in SEQ ID NO: 60 and CDR-H3 comprising or consisting of the amino acid sequence Trp-Gly-Arg and a variable light (VL) chain sequence comprising CDR-L1 as defined in SEQ ID NO: 62, CDR-L2 as defined in SEQ ID NO: 63 and CDR-L3 as defined in SEQ ID NO: 64; or
b) is an antibody binding to the same epitope as an antibody of (a).

The inventive binding moieties/antibodies provide valuable insights into how antibodies against antigens that are known as “immunologically inert” (like PAS sequences) can be obtained via immunization approaches as provided herein. Furthermore, the present invention also provides for means and methods how binding moieties/antibodies that specifically bind to and/or recognize “feature-less peptides” lacking pronounced hydrophobic or charged side chains and/or without defined secondary structure and/or comprising a random coil conformation or configuration may be obtained. The binding moieties/antibodies provided in the context of this invention and as characterized herein also offer valuable tools for the preclinical and clinical development of drug conjugates, like PASylated biologics or PASylated (small molecule) drugs—“PASylated” meaning conjugated with a PAS molecule/sequence/(poly)peptide.

Exemplary PASylated proteins or peptides include but are not limited to adenosine deaminase, agalsidase alfa, alpha-human atrial natriuretic peptide, amylin or analogs, anti-HIV fusion inhibitor (like enfurvitide), asparaginases (like calaspargase), B domain deleted factor VIII (like beroctocog alfa or octofactor), bacteriolysins including endolysins and ectolysins, bicyclic peptides (like TG-758), bradykinin antagonist (like icatibant), brain natriuretic peptide (BNP or B-type natriuretic peptide), calcitonin, CD19 antagonist, CD20 antagonist (like rituxan), CD3 receptor antagonist, CD40 antagonist, CD40L antagonist (like dapirolizumab or Antova), cerebroside sulfatase, chorionic gonadotropin, coagulation factor IV, coagulation factor IX, coagulation factor VIIa (like eptacog alfa), coagulation factor VIII (like susoctocog alfa), coagulation factor Xa, coagulation factor XIII (like catridecacog), complement component 5a antagonist, complement factor C3 inhibitor, C-peptide, Crisantaspase, CTLA-4 antagonist, C-type natriuretic peptide, deoxyribonuclease I (like dornase alfa), EGFR receptor antagonist, erythropoietin (like erythropoietin alfa or erythropoietin zeta), exendin-4, exendin-4 analog (like exendin 9-39), Fc gamma IIB receptor antagonists, fibroblast growth factor 1 (human acidic fibroblast growth factor), fibroblast growth factor 18, fibroblast growth factor 2 (human basic fibroblast growth factor), fibroblast growth factor 21, fibroblast growth factor receptor 2 antagonists (like FPA144), follicle-stimulating hormones (like follitropin alfa or follitropin beta), gastric inhibitory polypeptide (GIP), GIP analog, GLP-1, GLP-1 analog (like lixisenatide, liraglutide or semiglutide), GLP-2, GLP-2 analog (like teduglutide), glucagon or analogs, glucocerebrosidase (like imiglucerase), gonadorelin, gonadotropin-releasing hormone agonist (like goserelin, buserelin, triptorelin, leuprolide, protirelin, lecirelin, fertirelin or desiorelin), gonadotropin-releasing hormone antagonist (like abarelix, cetrorelix, degarelix, ganirelix or teverelix), gp120, gp160, granulocyte colony stimulating factor (G-CSF), granulocyte macrophage colony stimulating factor (GM-CSF), grehlin, growth hormone (like human, feline, bovine or porcine growth hormone), hematide, hepatocyte growth factor, hepcidin antagonist, hsp70 antagonist, human chorionic gonadotropin (like choriogonadotropin alfa), human parathyroid hormone, hyalosidase or bovhyaluronidase, hyaluronidase (like human hyaluronidase PH-20), glucocerebrosidase), iduronate-2-sulfatase, insulin, insulin analog, insulin like growth factor 1, insulin-like growth factor 2, integrin α4β1 antagonist, interferon tau, interferon-alpha, interferon-alpha antagonist, interferon-alpha superagonist, interferon-alpha-n3 (like Alferon N Injection), interferon-beta, interferon-gamma, interferon-lambda, interleukin, interleukin 2 fusion protein (like DAB(389)IL-2), interleukin receptor antagonist (like interleukin-1 receptor antagonists, EBI-005 or anakinra), interleukin-11 (like oprelevkin), interleukin-12, interleukin-17 receptor antagonist, interleukin-18 binding protein, interleukin-2, interleukin-22, interleukin-22 receptor subunit alpha (IL-22ra) antagonist, interleukin-38 (IL-38), interleukin-4, interleukin-6 receptor antagonis, interleukin-7, kynureninase, L-arginine degrading enzymes (like arginase or arginine deiminase), leptin, L-iduronidase, L-phenylalanine degrading enzyme (like phenylalanine hydroxylase or phenylalanine ammonia lyase), N-acetylgalactosamine-6-sulfatase (like elosulfase alfa), Nanofitins, neutrophil gelatinase-associated lipocalin, Anticalins, octreotide, Ornithodoros moubata complement inhibitor (OmCI/Coversin), parathormone (PTH), PD1 antagonist, PD1L antagonist, PDGF antagonist, (PYY 3-36), phenylalanine ammonia lyase (like valiase), Phylomers, platelet derived growth factor, relaxin, RGD peptide, serine protease inhibitors (like conestat alfa), soluble CD64, soluble DCC (deleted in colorectal cancer) receptor, soluble Fc-receptor (like CD16, CD32, CD64), soluble tumor necrosis factor I receptor (sTNF-RI), soluble tumor necrosis factor II receptor (sTNF-RII), soluble VEGF receptor, somatostatin, somatostatin analog (like pasireotide or CAP-232), stresscopin, T-cell receptor ligand, teriparatide (PTH 1-34), thymosin alpha 1, thymosin beta 4, thymosin beta 15, tumor necrosis factor (TNFalpha), tumor necrosis factor alpha antagonist, uricase (like rasburicase or pegadricase), urocortin, vasoactive intestinal peptide, vasopressin, vasopressin analog (like desmopressin, felypressin or terlypressin), VEGF antagonist (like ranbizumab or bevacizumab), VEGF antagonist, Adnectins, PDGF antagonist, DARPins, von Willebrand factor (like vonicog alfa).

Exemplary PASylated small molecule drugs include but are not limited to amanitin, auristatin, calicheamicin, camptothecin, digoxigenin, fluorescein, doxorubicin, fumagillin, dexamethasone, geldanamycin, paclitaxel, docetaxel, irinotecan, cyclosporine, buprenorphine, naltrexone, naloxone, vindesine, vancomycin, risperidone, aripiprazole, palonosetron, granisetron, cytarabine, nucleic acids (like antisense nucleic acids), small interfering RNAs (siRNAs), micro RNA (miR) inhibitors, microRNA mimetics, DNA aptamers, RNA aptamers, LNA (locked nucleic acid), RNA vaccines, DNA vaccines, carbohydrates suitable for the preparation of vaccines, for example tumor-associated carbohydrate antigens (TACA, α-GalNAc—O-Ser/Thr), sialyl Tn antigens (e.g. NeuAcα(2,6)-GalNAcα-O-Ser/Thr), Thomsen-Friedenreich antigen (Galβ1-3GalNAcα1), Lewis Y (e.g. Fucα(1,2)-Galβ(1,4)-[Fucα(1,3)]-GalNAc), sialyl-Lewis X or sialyl-Lewis A.

In specific contexts, for example for research purposes, as diagnostic tools, in screening methods, including patient stratification, etc., it may be useful that the inventive antigen-binding molecule, in particular the antibody or an antigen-binding fragment thereof, comprises a tag and/or a label. Accordingly, the present invention also relates to the antigen-binding molecule/antibody/antigen-binding fragment thereof as obtainable by the means and methods of the present invention and/or as provided herein, wherein said antigen-binding molecule/antibody/antigen-binding fragment thereof is conjugated or fused to (a) reporter molecule(s), (a) tag(s) and/or (a) label(s). Such reporter molecules, tags and/or labels are very well known in the art and may, inter alia, comprise small molecule fluorescent dyes, for example applied in a chemically activated manner (including N-hydroxysuccinimide ester, isothiocyanate, iodoacetate or maleimide), such as xanthene derivatives, e.g. fluorescein, rhodamine, Alexa dyes like Alexa488, cyanine derivatives such as Cy3 or Cy5, organoboron compounds such as 4,4-difluoro-4-bora-3a,4a-diaza-s-indacene (BODIPY), small molecules (haptens) such as biotin or digoxigenin, fluorescent proteins such as the green fluorescent protein or its derivatives, the red fluorescent protein or its derivatives or allophycocyanin, enzymes such as alkaline phosphatase, horseradish peroxidase or enzymes catalyzing visible light emission (bioluminescence) such as luciferases.

The present invention also provides a polynucleotide that encodes at least one of a variable heavy (VH) chain sequence and/or a variable light (VL) chain sequence of an antigen-binding molecule, in particular the antibody or the antigen-binding fragment of this invention. In a preferred embodiment of this invention, said polynucleotide encodes at least one of a variable heavy (VH) chain sequence and/or a variable light (VL) chain sequence of an antigen-binding molecule, in particular an antibody or an antigen-binding fragment, capable of specifically binding to Pro/Ala-rich sequences (PAS) and/or to amino acid sequences consisting of at least 4 or at least 10 or at least 20 amino acid residues forming random coil conformation, and whereby said amino acid residues forming said random coil conformation are selected from Pro (P), Ala (A) and Ser (S) or are Pro (P) and Ala (A), or that is capable of specifically binding to an antigenic portion thereof. The inventive polynucleotide may encode an antigen-binding molecule (or a fragment thereof) that is capable of binding to an epitope of the structure:
(P/S)A(A/S)P and/or
PA(A/S)P.

The polynucleotide of the invention preferably encodes at least one of a variable heavy (VH) chain sequence and/or at least one of a variable light (VL) chain sequence of an antigen-binding molecule, in particular the antibody or the antigen-binding fragment as provided herein. Preferably, said antigen-binding molecule, in particular the antibody or the antigen-binding fragment, binds an epitope on an intrinsically disordered protein and/or on a intrinsically disordered protein domain or peptide. Preferably, said intrinsically disordered protein and/or on an intrinsically disordered protein domain or peptide comprises or consist of Pro/Ala-rich sequences (PAS). Said epitope may comprises an epitope/epitope stretch as disclosed herein and may be selected from the group consisting of PAPAAP (SEQ ID NO: 8), PAPASP (SEQ ID NO: 9), PASPAAP (SEQ ID NO: 10), PSAAPS (SEQ ID NO: 79), ASPAAP (SEQ ID NO: 80), PASPAA (SEQ ID NO: 81), PAAP (SEQ ID NO: 82), PASP (SEQ ID NO: 83), APSA (SEQ ID NO: 84) and PSAA (SEQ ID NO: 85).

Corresponding polynucleotides/nucleic acid molecules, including DNA or RNA, may readily be obtained via routine sequencing methods known to the skilled artisan and as also illustrated in the appended examples. As a source of such sequencing techniques, B-cells from the non-human animals immunized in accordance with the method of the present invention may be used. Such cells comprise “hybridoma cells” that can be produced without further ado, for example as illustrated in manuals for the generation of monoclonal antibodies, like (Harlow & Lane, 1988). Examples for such inventive polynucleotides/nucleic acid molecules, including DNA or RNA, are the polynucleotides/nucleic acid molecules, including DNA, as comprised in the deposited clones DSM ACC3365, DSM ACC3366 or DSM ACC3367. These deposited clones are hybridomas which comprise polynucleotides capable of encoding the illustrative monoclonal antibodies (Anti-PA(S) MAbs) of the invention, Anti-PA(S)Mab 1.1, Anti-PA(S)Mab 2.1 and Anti-PA(S)Mab 3.1, respectively.

As is evident from the enclosed deposit receipts, these three hybridomas have been deposited under the stipulations of the Budapest Treaty on Nov. 13, 2020 (2020-11-13) at the “DSMZ” (Leibnitz-Institut DSMZ—Deutsche Sammlung von Mikroorganismen und Zellkulturen GmbH) and have received the accession numbers from said International Depository Authority: DSM ACC3365 (Anti-PA(S)Mab 1.1); DSM ACC3366 (Anti-PA(S)Mab 2.1) and DSM ACC3367 (Anti-PA(S)Mab 3.1).

The present invention also relates to these deposits and, accordingly, to the hybridomas DSM ACC3365, DSM ACC3366 and DSM ACC3367.

The invention also relates to a host cell comprising the polynucleotide of the invention, i.e. a polynucleotide encoding at least one of a variable heavy (VH) chain sequence and/or a variable light (VL) chain sequence of an antigen-binding molecule, in particular the antibody or the antigen-binding fragment, of this invention. The inventive (host) cell may also be a cell that expresses the polynucleotide as comprised in a hybridoma as provided herein, like, DSM ACC3365, DSM ACC3366 or DSM ACC3367. Said hybridomas may also be the host cell of the present invention.

Also provided herein is a method for producing an antigen-binding molecule, in particular the antibody or the antigen-binding fragment of this invention, comprising culturing the hybridoma of the invention and/or comprising culturing the host cell, for example a bacterial cell or a mammalian cell, of the invention. Said production of said inventive antigen-binding molecule may comprise routine culturing of the host cells and/or hybridomas of the invention. Further hybridomas producing the antigen-binding molecule, in particular the antibody or the antigen-binding fragment, of this invention may be obtained without further ado by the means and methods provided herein for generating binding moieties, in particular antigen-binding molecules, directed against and/or specifically binding to intrinsically disordered proteins and/or intrinsically disordered protein domains or peptides as defined herein. The method of production of the inventive antigen-binding molecule may also comprise the isolation or purification of said antigen-binding molecule form the culturing system, for example from the culturing broth of the host cells/hybridomas.

In one embodiment the invention also provides for a method for producing an antibody that specifically binds to a Pro/Ala-rich sequence (PAS) as defined herein or to an antigenic portion thereof, said method comprising administering to a non-human mammal a Pro/Ala-rich sequence (PAS) and/or
an amino acid sequence consisting of at least 20, preferably 40 amino acid residues forming random coil conformation, whereby said amino acid residues forming said random coil conformation are selected from Pro (P), Ala (A) and Ser (S) or are Pro (P) and Ala (A), or to an antigenic portion thereof,
   (i) wherein said Pro/Ala-rich sequence (PAS) and/or wherein said amino acid sequence consisting of at least 20, preferably 40 amino acid residues forming random coil conformation comprises at least one epitope/epitope stretch of the structure:
      (P/S)A(A/S)P and/or
      PA(A/S)P,
   (ii) wherein said Pro/Ala-rich sequence (PAS) and/or wherein said amino acid sequence consisting of at least 20, preferably 40 amino acid residues forming random coil conformation comprises a protecting group which is attached N-terminally; and
   (iii) wherein an immunoadjuvant is linked C-terminally to said Pro/Ala-rich sequence (PAS) and/or said amino acid sequence consisting of at least 20, preferably 40 amino acid residues forming random coil conformation.

In one embodiment, in said method for producing an antibody that specifically binds to a Pro/Ala-rich sequence (PAS) as recited above, said epitope of (i) comprises an epitope/epitope stretch selected from the group consisting of PAPAAP (SEQ ID NO: 8), PAPASP (SEQ ID NO: 9), PASPAAP (SEQ ID NO: 10), PSAAPS (SEQ ID NO: 79), ASPAAP (SEQ ID NO: 80), PASPAA (SEQ ID NO: 81), PAAP (SEQ ID NO: 82), PASP (SEQ ID NO: 83), APSA (SEQ ID NO: 84) and PSAA (SEQ ID NO: 85). In a further embodiment that epitope/epitope stretch is comprised in a peptide defined herein above as R^{N}—(P/A)-R^{C}.

The invention also relates to a composition comprising the binding moiety(ies), in particular antigen-binding molecule(s), as generated by the means and methods of the present invention that are capable of specifically binding intrinsically disordered protein domains or peptides. The claimed composition may also comprise binding moiety(ies), in particular antigen-binding molecule(s) as obtainable by said inventive methods as well as to binding moiety(ies), in particular to antigen-binding molecule(s), that were produced by the methods provided herein above.

Also comprised in the present invention are compositions that comprise the specific antigen defined herein, which is a conjugate of an immunoadjuvant and one or more P/A peptides as defined above, wherein each of said P/A peptide(s) may be independently a peptide of the structure R^{N}—(P/A)-R^{C}.

The inventive binding moiety(ies), in particular antigen-binding molecule(s)/antibodies or antigen-binding fragments thereof, are particularly useful as research tools and as bioanalytical tools. They may be used also for the in vitro screening of patient samples, like blood samples obtained from individuals that have been treated with PASylated drugs and/or proteins. On the other hand, also samples obtained from individuals who have never received PASylated drugs and/or proteins may be tested in vitro with the binding moiety(ies), in particular antigen-binding molecule(s)/antibodies or antigen-binding fragments thereof, of the present invention. This may be considered as “negative control” and may be helpful to assess or avoid false positive reactions of antibodies of the present invention. Accordingly, the compositions of the present invention, in particular the compositions comprising the antigen-binding molecule(s)/antibodies or antigen-binding fragments thereof, may be useful in (patient) screenings and/or for following the time course of a (concomitant) treatment of said patient/individual with PASylated (small molecule) drugs and/or protein/peptide drugs. Accordingly, the present invention also relates to diagnostic compositions.

In accordance with the above, the present invention also provides a method of detecting
(i) a Pro/Ala-rich sequence (PAS),
(ii) a conjugate of a protein, peptide or small molecule drug and Pro/Ala-rich sequence (PAS), and/or
(iii) a conjugate of a protein, peptide or small molecule drug and an amino acid sequence consisting of at least 20 amino acid residues forming random coil conformation and whereby said amino acid residues forming said random coil conformation are selected from Pro (P), Ala (A) and Ser (S) or are Pro (P) and Ala (A)
   in a biological sample.

Accordingly, the claimed method may be an in vitro method using a biological sample that was obtained from an individual, in particular from a mammal, preferably from a human, treated or supposed to be treated with PASylated drugs and/or proteins/peptides.

Said in vitro method may comprise contacting said biological sample with the antigen-binding molecule and/or an antibody of the present invention under conditions permissive for binding of the antigen-binding molecule and/or antibody to said Pro/Ala-rich sequence (PAS) of (i) or (ii) and/or to said amino acid residues forming said random coil conformation of (iii). Said method may also comprise as additional step the detection whether a complex is formed between said antigen-binding molecule and/or said antibody and said Pro/Ala-rich sequence (PAS) and/or said amino acid residues forming said random coil conformation. A (positive) detection of the Pro/Ala-rich sequence (PAS) and/or said amino acid residues forming said random coil conformation in said biological sample may be indicative whether e.g. a drug/protein that comprises a Pro/Ala-rich sequence (PAS), i.e. a “PASylated (small molecule) drug and/or protein or peptide drug”, is still present in the individual's body. This would be a qualitative assay. However, time courses and and/or quantification of drug/protein that comprises a Pro/Ala-rich sequence (PAS) in these biological samples are envisaged, too. Such assays also comprise “screening assays” of the individuals' biological samples.

The detection of the complexes formed between the inventive binding moiety(ies), in particular antigen-binding molecule(s)/antibodies or antigen-binding fragments thereof, and said Pro/Ala-rich sequence (PAS) and/or said conjugates of a protein/peptide drug/small drug comprising such Pro/Ala-rich sequence (PAS) in vitro is routine work for the skilled artisan. Such detection of the formed complexes may comprise known techniques like immunohistochemistry, immunofluorescence imaging, enzyme-linked immunosorbent assay (ELISA), western blotting, electrochemiluminescence (ECL) immunoassay (ECLIA), surface plasmon resonance (SPR, Biacore), lateral flow Immunoassay, paper-based immunoassay, acoustic wave-based immunoassay, interferometry-based Immunoassay, nanomaterial and micromaterial-based immunoassay, microcantilever-based sensor, quartz crystal microbalance-based sensor, electrochemical immunosensor, Lab-on-a-Chip (LOC) immunoassay, smartphone-based immunoassay, mass spectrometry based immunoassay (MSIA, Immuno-MALDI, Immuno-MRM, SISCAPA) or immunoprecipitation. Also envisaged are radiographic methods and imaging, for example after corresponding labeling of the inventive binding moiety(ies) with a radioactive substance as well known in the art.

It is also envisaged that the binding moiety(ies), in particular antigen-binding molecule(s)/antibodies or antigen-binding fragments thereof, are used in vivo on individuals, for example in a research setting whereby non-human animals are tested and screened with these inventive compounds and compositions.

In accordance with the above, the present invention also relates to a method for monitoring the response to treatment of a subject or an animal with a PASylated drug conjugate, said method comprising the use of an antigen-binding molecule and/or an antibody or a composition of the invention, for and/or in measuring the level of circulating Pro/Ala-rich sequence (PAS) molecules and/or fusion proteins and/or drug conjugates comprising Pro/Ala-rich sequence (PAS) molecules in a blood sample, preferably a plasma or serum sample, at one or more time points before and at one or more time points after treatment of the subject/patient or a non-human test individual, with
(a) a conjugate of a protein or peptide or small molecule drug with a Pro/Ala-rich sequence (PAS) and/or
(b) a conjugate of a protein drug or peptide or small molecule with an amino acid sequence consisting of at least 20 amino acid residues forming random coil conformation and whereby said amino acid residues forming said random coil conformation are selected from Pro (P), Ala (A) and Ser (S) or are Pro (P) and Ala (A).

This method may also comprise detection of a time course and/or a time-dosing relationship, in particular when samples are screened that are taken at different time points after said treatment of said subject/patient or said non-human test individual with any of the conjugates defined in (a) or (b), supra.

Also, the detection of the complexes formed between the inventive binding moiety(ies), in particular antigen-binding molecule(s)/antibodies or antigen-binding fragments thereof, and said Pro/Ala-rich sequence (PAS) and/or said conjugates of a protein drug/small drug comprising such Pro/Ala-rich sequence (PAS) is routine work and the embodiments provided herein above also apply for this “method of monitoring” mutatis mutantis.

The invention is further described by the following non-limiting figures and examples.

### FIGURES

FIG. 1: Amino acid sequence alignment for the V_{H} (A) and V_{L} (B) domains of the anti-PAS antibodies of this invention (prior to the introduction of flanking restriction sites for subcloning). CDRs are labelled with a black outline. Identical amino acid positions with regard to the V_{H} and V_{L} sequences of Anti-PA(S) MAb 1.1 shown at the top of each alignment are depicted as “.”, gaps in the amino acid sequence alignment are indicated by “-”.

FIG. 2: Exemplary western blot analysis demonstrating that the different anti-PAS antibodies of this invention bind specifically to the corresponding PASylated fusion proteins. Western blots incubated with cell culture supernatant from hybridoma clones (A) Anti-PA(S) MAb 2.2, (B) Anti-PA(S) MAb 2.1, (C) purified Anti-PA(S) MAb 2.1, (D) Anti-PA(S) MAb 3.1, (E) Anti-PA(S) MAb 3.2, (F) Anti-PA(S) MAb 1.1, (G) Anti-PA(S) MAb 1.2 and (H) the anti-mouse IgG Fc-specific alkaline phosphatase (produced in goat, Sigma-Aldrich) secondary antibody as control. The following samples were applied to SDS-PAGE for western blotting: M—PageRuler Prestained Protein Ladder, 10 to 180 kDa (Thermo Fisher Scientific); 1—PAS #1(200)-IL1Ra (SEQ ID NO: 72); 2—P/A #1(200)-IL1Ra (SEQ ID NO: 73); 3—APSA(200)-IL1Ra (SEQ ID NO: 74); 4—pooled human serum (SEQENS IVD/H2B), 1:200 diluted in ddH₂O and spiked with 1 μg IL1Ra (Kineret/Anakinra, SOBI); 5—E. coli BL21 whole cell protein, lysed in SDS sample buffer.

FIG. 3: Exemplary results of ELISA experiments to detect PAS sequences or PASylated fusion proteins with the anti-PAS antibodies of this invention: (A) ELISA with the Fab fragment of Anti-PA(S) MAb 2.1 and IL1Ra-PAS #1(800), PAS #1(600)-Leptin and P/A #1(600)-GMCSF as test substances as well as BSA as control. (B) ELISA with the Fab fragment of Anti-PA(S) MAb 2.2 and P/A #1(600) as test substance. (C) ELISA with the Fab fragment of Anti-PA(S) MAb 1.2 and IL1Ra-PAS #1(800), PAS #1(600)-Leptin and P/A #1(600)-GMCSF test substances as well as BSA as control. (D) ELISA with hybridoma supernatant of Anti-PA(S) MAb 1.2, captured using an anti-mouse IgG Fc specific from goat pre-adsorbed to the microtiter plate, and hu4D5-PAS #1(200) as test substance.

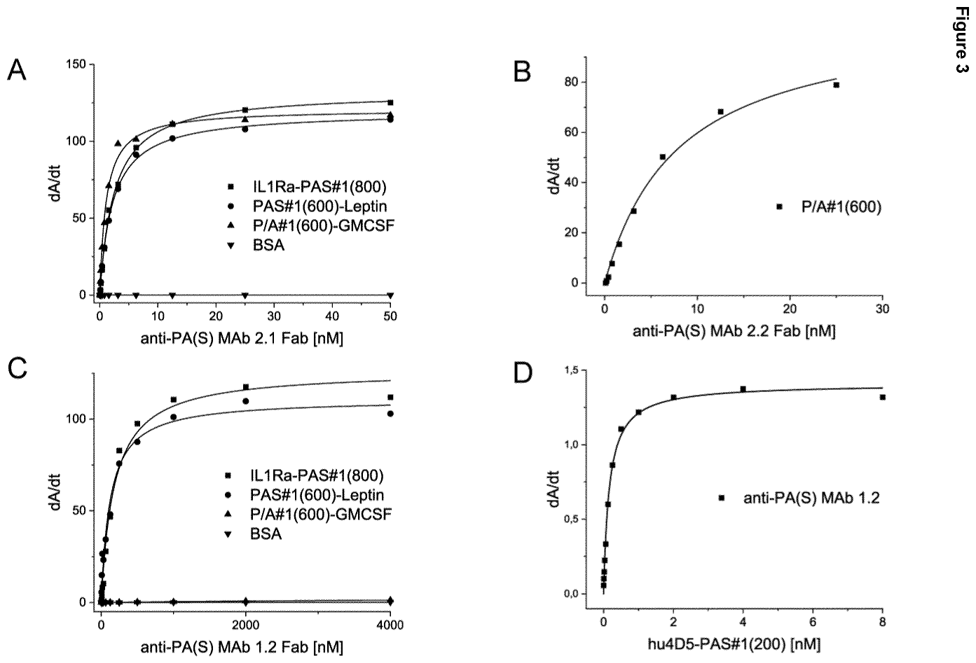

FIG. 4: Exemplary results of ELISA experiments to detect PAS sequences or PASylated fusion proteins with the anti-PAS antibodies of this invention: (A) ELISA with the Fab fragment of Anti-PA(S) MAb 3.1 and APSA(200)-IL1Ra as test substance. (B) ELISA with the Fab fragment of Anti-PA(S) MAb 3.2 and APSA(200)-IL1Ra as test substance. (C) ELISA with the Fab fragment of Anti-PA(S) MAb 1.1 and PAS #1(600)-Leptin as test substance. (D) MAb capture ELISA (see FIG. 3D) with hybridoma supernatant of Anti-PA(S) MAb 2.1 and hu4D5-P/A #1(200) as test substance.

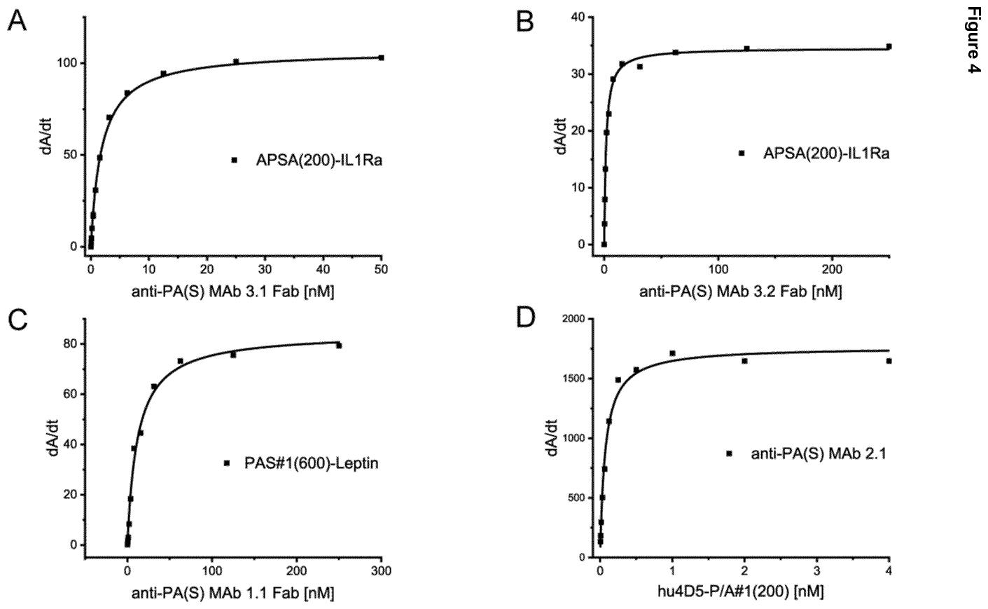

FIG. 5: SPOT assay results for the hybridoma culture supernatant of Anti-PA(S) MAb 1.1 and Anti-PA(S) MAb 1.2. Consecutive 12-mer peptides from the PAS #1 and P/A #1 sequences, each shifted by one residue in SEQ ID NOs: 5 and 6, respectively, were synthesized C-terminally anchored on a hydrophilic membrane. After color development of the membrane, the spot intensities were scanned and quantified with the software CLIQS ver. 1.2.044 (TotalLab) and are displayed as bar graphs. Epitope sequences are highlighted in bold.

FIG. 6: SPOT assay results for the Fab fragment of Anti-PA(S) MAb 2.1 and the hybridoma culture supernatant of Anti-PA(S) MAb 2.2. For explanation, see FIG. 5.

FIG. 7: SPOT assay results for the hybridoma culture supernatants of Anti-PA(S) MAb 3.1 and Anti-PA(S) MAb 3.2. A 10-mer peptide comprising the sequence AAPSAAPSAA was synthesized C-terminally anchored on a hydrophilic membrane whereby positions 3 to 8 were consecutively substituted by all twenty proteinogenic amino acids. After color development of the membrane, the spot intensities were scanned and quantified with the software CLIQS ver. 1.2.044 (TotalLab) and are displayed as bar graphs. Bars corresponding to the residue in the original sequence AAPSAAPSAA are filled.

FIG. 8: Exemplary SPR sensorgrams for Anti-PA(S) MAb 3.1 (APSA(200)-IL1Ra as analyte) as well as Anti-PA(S) MAb 1.1 (PAS #1(200)-IL1Ra as analyte) and Anti-PA(S) MAb 1.2 (PAS #1(200)-IL1Ra as analyte measured on a Biacore X 100 instrument). Hybridoma supernatants were applied to a CM3 sensorchip (GE Healthcare) coated with an anti-mouse antibody (Mouse Antibody Capture Kit; GE Healthcare). Injection phases are labeled with black bars, together with the corresponding concentration of injected analyte.

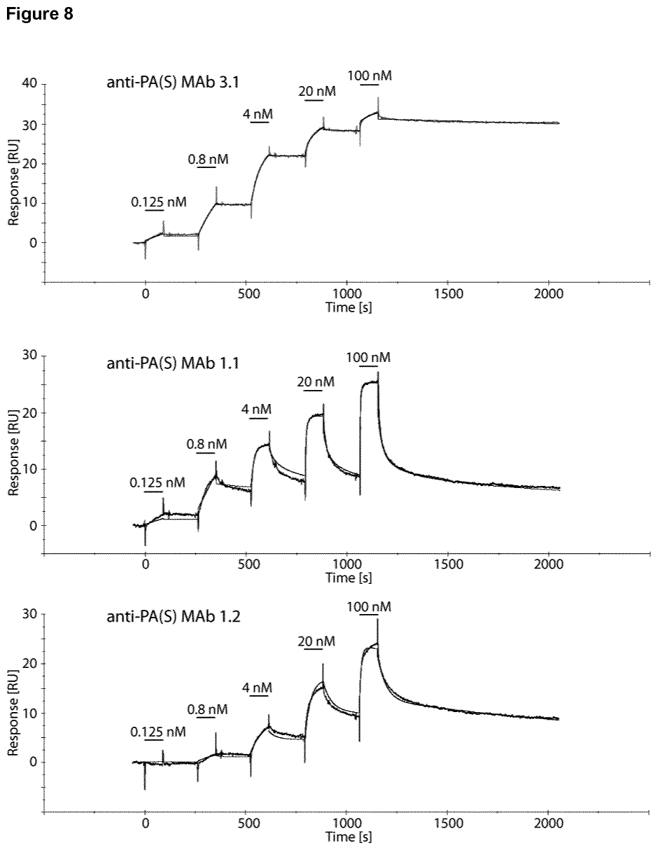

FIG. 9: Principle of the affinity purification of PASylated proteins using a column with immobilised anti-PAS Fab 1.2. (A) Schematic illustration of the one-step purification of a PASylated protein: (i) application of the cell extract containing the PASylated protein of interest, (ii) column washing with running buffer and (iii) elution of the PASylated protein of interest by applying a 1 M solution of L-prolinamide in running buffer. (B) Crystal structure (PDB ID: 7031) of Fab 1.2 (cartoon diagram) in complex with its PAS #1 epitope peptide (shown as stick model at the top). (C) Relevant part of the PAS #1 peptide epitope recognized by Fab 1.2. (D) Chemical structure of L-prolinamide.

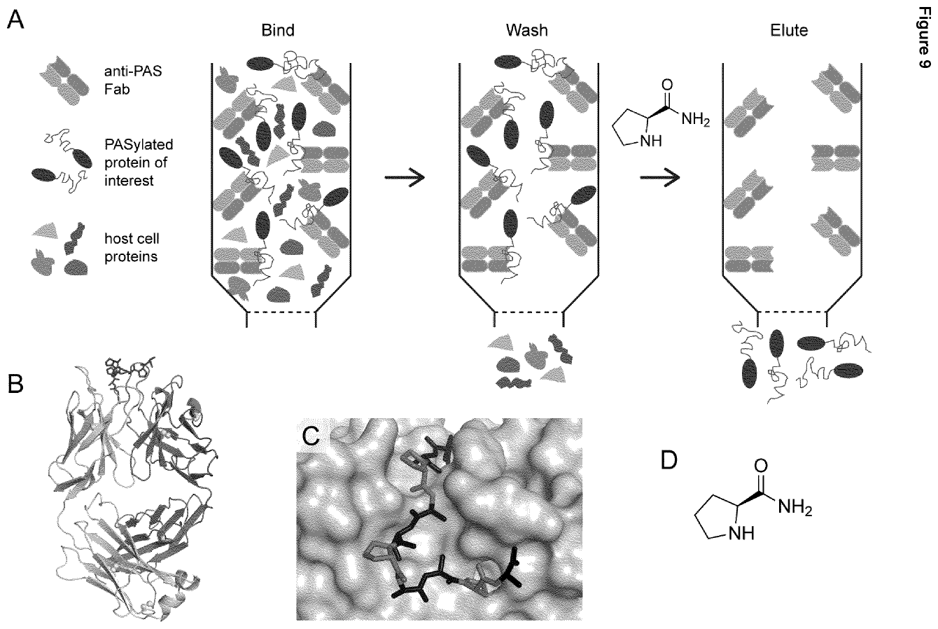

FIG. 10: Exemplary chromatograms of the affinity purification of a PASylated protein using an immobilized anti-PAS antibody of this invention. The Fab fragment of Anti-PA(S) MAb 1.2 was covalently immobilized to a 1 ml HiTrap HP column (GE Healthcare) to serve as affinity matrix. The StrepII-eGFP-PAS #1(200) fusion protein (SEQ ID NO: 71) was applied as a test protein for purification from (A, C) a previously purified protein solution and (B, D) a whole cell extract of BL21 E. coli cells expressing StrepII-eGFP-PAS #1(200). The protein elution from the chromatography column was monitored by measuring the general absorbance at 280 nm and also the specific absorbance of the eGFP chromophore at 488 nm in parallel (A, B). After application of the protein sample and washing with buffer, the bound PAS fusion protein was eluted with a 1 M solution of L-prolinamide, which also showed strong absorbance at 280 nm (possibly due to contamination with aromatic amino acids). SDS-PAGE analysis (C, D) revealed the presence of StrepII-eGFP-PAS #1(200) in these elution fractions. Samples: M—Pierce Unstained Protein MW Marker (Thermo Fisher Scientific); 0—pure StrepII-eGFP-PAS #1(200); 1—fractions 7-7.5 ml; 2—fractions 7.5-8 ml; 3—fractions 8-8.5 ml; 4—fractions 8.5-9 ml; 5—fraction 9-9.5 ml; 6—fractions 9.5-10 ml; 7—fractions 10-10.5 ml. 1*—whole cell lysate of BL21 E. coli cells expressing StrepII-eGFP-PAS #1(200); 2*—fractions 2-4 ml; 3*—fractions 7-7.5 ml; 4*—fractions 7.5-8 ml; 5*—fractions 8-8.5 ml; 6*—fractions 8.5-9 ml; 7*—fractions 9-9.5 ml; 8*—fractions 9.5-10 ml; 9*—fractions 10-10.5 ml. This experiment demonstrates that the anti-PAS affinity column specifically binds the PASylated test protein, StrepII-eGFP-PAS #1(200), which can be eluted under mild conditions by applying a solution of L-prolinamide.

FIG. 11: Exemplary chromatograms and SDS PAGE analysis documenting the one-step PAS affinity purification of therapeutically relevant PASylated proteins using an immobilized anti-PAS antibody of this invention. The Fab fragment of Anti-PA(S) Mab 1.2 was covalently immobilized to a 1 ml HiTrap HP column (GE Healthcare) to serve as affinity matrix. The C-terminally PASylated Anticalin H1GA-PAS #1(200)-His₆ (SEQ ID NO: 90) (A, B) and the N-terminally PASylated cytokine PAS #1(800)-IL1 Ra (SEQ ID NO: 91) (C, D) were purified from either the periplasmic or from the cell fraction of E. coli BL21 respectively. Protein elution from the chromatography column was monitored by measuring the absorbance at 280 nm. After application of the protein sample and washing with buffer, the bound PAS fusion proteins were eluted with a 1 M solution of L-prolinamide in running buffer. SDS-PAGE analysis (B, D) revealed the presence of the PASylated proteins in the elution fraction. Samples: Unstained Protein MW Marker (Thermo Fisher Scientific)-PE/CE (periplasmic extract/whole cell extract of E. coli BL21 cells expressing the respective proteins)-FT (flow through)-Wash-Elution. Arrows indicate the protein bands corresponding to H1GA-PAS #1(200)-His₆ and PAS #1(800)-IL1Ra respectively. This experiment demonstrates that N- and C-terminally PASylated proteins carrying PAS tags comprising 200-800 residues can be purified with high selectivity.

FIG. 12: Exemplary chromatogram of the affinity purification of a PASylated protein using an immobilized anti-PAS antibody of this invention. The Fab fragment of Anti-PA(S) MAb 1.2 was covalently immobilized to a 1 ml HiTrap HP column (GE Healthcare) to serve as affinity matrix. The pre-purified StrepII-eGFP-PAS #1(200) fusion protein (SEQ ID NO: 71) was applied as a test protein. Protein elution from the chromatography column was monitored by measuring the specific absorbance of the eGFP chromophore at 488 nm. After application of the protein sample and washing with buffer, the bound PAS fusion protein was eluted under particularly mild conditions (1 M L-prolinamide, 100 mM Tris, 150 mM NaCl, 1 mM EDTA, pH adjusted to 8.0 with HCl). This experiment demonstrates that the anti-PAS affinity column quantitatively binds the PASylated test protein, StrepII-eGFP-PAS #1(200), which can be eluted under mild buffer conditions by applying L-prolinamide.

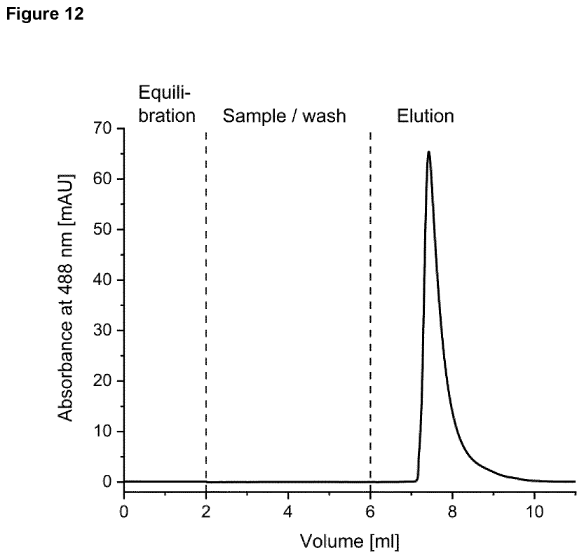

FIG. 13: Crystal structures of synthetic PAS epitope peptides in complex with recombinant Fab fragments of anti-PAS antibodies of the invention: (A) P/A #1-epitope peptide bound to Anti-PA(S) MAb 2.2, (B) PAS #1-epitope peptide bound to Anti-PA(S) MAb 1.1, (C) Anti-PA(S) MAb 1.2 and (D) Pga-(APSA)₃ peptide bound to Anti-PA(S) MAb 3.1. Epitope peptides are shown as sticks (dark gray), Fab heavy (middle grey) and light (light grey) chains are shown as wires.

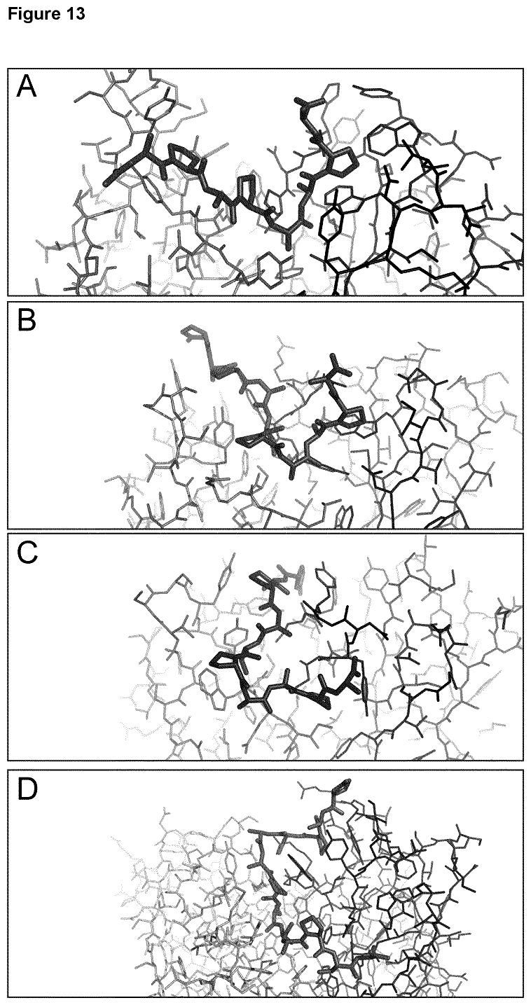

FIG. 14: Pharmacokinetic (PK) study of PASylated Thymosin alpha 1 in Wistar rats. (A) Linear range of the standard curve used for quantification of PASylated Thymosin alpha 1 in rat plasma samples via ELISA setup A schematically illustrated in FIG. 15. (B) PASylated Thymosin alpha 1 was subcutaneously injected at a dose of 3.4 mg/kg body weight into female Wistar rats (N=5). The concentration of the fusion protein in plasma was quantified by a sandwich ELISA using Anti-PA(S) MAb 2.1 as capture antibody and the alkaline phosphatase conjugated Anti-PA(S) MAb 1.2 as detection reagent. Data were plotted against sampling time post injection and fitted using a one-compartment model. The PK profile shows distinct absorption and elimination phases of the PASylated peptide drug (for PK parameters see Table 1).

FIG. 15: Exemplary ELISA setups for detection of PASylated molecules. (A) Sandwich ELISA using an anti-PAS antibody adsorbed to a microtiter plate in order to capture a PASylated molecule and a second anti-PAS antibody conjugated to a reporter enzyme as detection reagent. (B) Sandwich ELISA using an anti-PAS antibody adsorbed to a microtiter plate in order to capture a PASylated molecule and a second enzyme-coupled antibody directed against the biological active moiety of the PASylated drug. (C) ELISA using a binding partner of the protein, peptide or small molecule drug (e.g. a receptor, here designated target) adsorbed to a microtiter plate in order to capture the PASylated molecule and an enzyme-coupled anti-PAS antibody of this invention to detect the PASylated drug. (D) Schematic illustration of a competitive ELISA showing a PASylated analyte molecule competing with a PASylated and biotinylated molecule for the binding site(s) of an anti-PA(S) MAb which is adsorbed to a microtiter plate. PASylated and biotinylated molecules bound to the MAb are subsequently detected by a streptavidin-enzyme conjugate.

FIG. 16: Fluorescence titration of the Fab fragment of Anti-PA(S) Mab 2.2, applied at 1 μM in 100 mM Tris/HCl pH 7.5, with the synthetic epitope peptide Abz-APAPAAPA. RFU—relative fluorescence units (fluorescence excitation at 280 nm, signal detection at 340 nm).

FIG. 17: Surface plasmon resonance (SPR) spectroscopy on a Biacore X100 instrument (Cytiva, Freiburg, Germany) using anti-PA(S) Mab 1.1. to capture a PASylated anti-Galectin Fab fragment and to determine the PAS-Fab binding kinetics towards its antigen Galectin-3. (A) Sensorgram showing the immobilization of a PASylated antibody fragment on a CM5 surface plasmon resonance (SPR) sensor chip (Cytiva). (1) The carboxylate groups on the dextran hydrogel surface of the chip were converted to reactive N-hydroxysuccinimide ester (NHS) groups using EDC/NHS chemistry. (2) Anti-PA(S) Mab 1.1 was covalently conjugated to the chip surface via the activated NHS esters, and (3) unreacted NHS-esters were saturated using 0.1 M ethanolamine. (B) Single cycle kinetic experiment showing (1) the non-covalent immobilization of a PASylated anti-Galectin Fab, followed by (2) five consecutive injections from a 1:2 dilution series (0.1 nM to 1.6 nM) of Galectin-3 as well as two acidic regeneration steps. (C) The baseline of the reference-corrected sensorgram of the single cycle kinetic experiment from (B) was set to zero as well as a start Time=0 s and fitted to a global 1:1 Langmuir binding model using the Biacore X100 evaluation software.

### EXAMPLES

### Example 1: Methods Employed in the Present Invention

### A. Preparation of PAS Peptide Conjugates for Immunization

Three different peptides were obtained by solid phase synthesis (Pga-PAS #1(40)-Ahx and Pga-P/A #1(40)-Ahx: Peptide Specialty Laboratories-PSL, Heidelberg, Germany; Pga-APSA(40)-Ahx: Almac Sciences, Edinburgh, Scotland), each with a blocked N-terminus:

Pga-PAS#1(40)-Ahx (Pga-ASPAAPAPASPAAPAPSAPA-
ASPAAPAPASPAAPAPSAPA-Ahx; SEQ ID NO: 5);
Pga-P/A#1(40)-Ahx (Pga-AAPAAPAPAAPAAPAPAAPA-
AAPAAPAPAAPAAPAPAAPA-Ahx; SEQ ID NO: 6);
Pga-APSA(40)-Ahx (Pga-APSAAPSAAPSAAPSAAPSA-
APSAAPSAAPSAAPSAAPSA-Ahx; SEQ ID NO: 7).

Pga means a pyroglutamyl residue (also known as 2-pyrrolidone-5-carboxylic acid or 5-oxoproline) and Ahx means aminohexanoic acid; all other residues are standard proteinogenic L-amino acids denoted by their single-letter abbreviations. The 40mer PAS peptides were designed with sufficient length in order to encompass at least two copies of the corresponding PAS sequence repeat, in some embodiments comprising 20 residues, thus also including at least one instance of the junction between two adjacent sequence repeats. Of note, such junctions would also constitute potential epitopes in longer recombinant PAS polypeptides. As all peptides contained chemically inert side chains only and had a blocked N-terminus, their single C-terminal carboxylate group (in fact, the one of the Ahx linker residue) was activated selectively and used for directed chemical conjugation to the ε-amino groups of Lys side chains of KLH, which was employed as a highly immunogenic carrier protein (Swaminathan et al., 2014). To this end, 50 mg of each peptide was dissolved in 1450 μl dimethylsulfoxide (DMSO) and activated with a 10 fold molar amount of each 2-(1H-benzotriazole-1-yl)-1,1,3,3-tetramethylaminium tetrafluoroborate (TBTU; Iris Biotech, Marktredwitz, Germany) and N,N-diisopropylethylamine (DIPEA; Sigma-Aldrich, Taufkirchen, Germany). 10 mg KLH (Thermo Scientific, Waltham, MA) was dissolved in water, dialyzed against PBS (4 mM KH₂PO₄, 16 mM Na₂HPO₄, 115 mM NaCl), adjusted to a concentration of 2.3 mg/ml in a volume of 4.35 ml and mixed with the activated peptide solution. After incubation on ice for 30 min, the solution was dialyzed against 25 mM Na-borate pH 9.0 and the conjugate was purified by anion exchange chromatography on a Source 15Q column (GE Healthcare, Munich, Germany) equilibrated with the same buffer. The conjugate was eluted in a linear concentration gradient of 0-500 mM NaCl applied in running buffer, monitored at 280 nm. Eluate fractions of the main peak were pooled, dialyzed against PBS, concentrated to 2 mg/ml, sterile-filtered through a 0.22 μm Millex-GV PVDF filter (Merck, Darmstadt, Germany) and flash-frozen in liquid nitrogen.

### B. Immunization of Mice and Generation of Hybridoma Cells

Using the PAS peptide-KLH conjugates described above as antigen, Balb/c mice were immunized and hybridomas were prepared according to standard procedures (ProMab Biotechnologies, Richmond, CA). For each antigen, five Balb/c mice were immunized subcutaneously with 50 μg antigen together with Freund's complete adjuvant (CFA). Three weeks after priming, three booster injections (five for APSA(40)-KLH), each with 25 μg antigen and Freund's incomplete adjuvant (IFA), were applied at intervals of two weeks. A final boost with 50 μg of antigen without adjuvant was administered intraperitoneally two weeks after the last boost. Spleen cells were harvested from animals and fused with Sp2/0 myeloma cells for hybridoma clone generation using standard procedures well known in the art.

Promising hybridoma clones were propagated in cell culture using DMEM (Biochrom, Berlin, Germany) containing 10% v/v FCS (Ultra low IgG One Shot, Life Technologies, NY), 6 mM L-alanyl-L-glutamine (Biochrom), 1:100 penicillin/streptomycin (Biochrom) and supplemented with 10% v/v Hybridoma Premium Medium (ProMab Biotechnologies). Secreted anti-PAS MAbs in the cell culture supernatants were characterized by real-time surface plasmon resonance (SPR) spectroscopy and enzyme-linked immunosorbent assay (ELISA).

For some studies, Anti-PA(S) MAbs were purified from the hybridoma supernatants using a 1 ml HiTrap Protein G HP column (GE Healthcare) operated at a flow rate of 1 ml/min using an Akta Explorer 10 chromatography workstation (GE Healthcare). The hybridoma supernatant was diluted with binding buffer (20 mM NaPᵢ pH 7.0) at a 1:1 ratio and applied to the column, which had been pre-equillibrated with 10 column volumes of binding buffer. After washing with 10 column volumes of binding buffer, the antibody was eluted with 2 column volumes of elution buffer (0.1 M glycine/HCl pH 2.7). To preserve the activity of acid-labile IgGs, 200 μl of 1 M Tris/HCl pH 9.0 per 1 ml collection volume were added to each collection tube prior to the fractionation. Fractions containing the affinity-purified MAb were subsequently dialyzed against 200 volumes of storage buffer (20 mM KPi, 125 mM NaCl, 50% glycerol, pH 7.2) and frozen at −21° C. Protein concentration was determined by measuring the absorbance at 280 nm (A₂₈₀=1.4 equalling a concentration of 1.0 mg/ml IgG).

### C. Characterization of Hybridoma MAbs by ELISA and SPR

Characterization of hybridoma MAbs by ELISA was performed using NUNC Maxisorp F 96-well plates (Thermo Fisher Scientific, Munich, Germany) coated with 50 μl of a 5 μg/ml solution of anti-mouse IgG Fc-specific goat antibody (Sigma-Aldrich) in PBS for 1 h, followed by twice washing with PBS and blocking with 3% w/v bovine serum albumin (BSA) in PBS/T (PBS+0.1% v/v Tween 20) for 1 h. After washing with PBS/T, the wells were incubated for 1 h with 50 μl of each hybridoma supernatant diluted 1:100 in PBS/T and washed again. Then, 50 μl solutions of the following PASylated proteins (each 8 nM) were applied in 1:2 dilution series with PBS and incubated for 1 h: hu4D5-PAS #1(200) (Schlapschy et al., 2013), hu4D5-P/A #1(200) (WO 2011/144756 A1) or APSA(200)-IL1Ra (SEQ ID NO: 74), which had been labeled with DIG-NHS (Santa Cruz Biotechnology, Dallas, TX) according to the manufacturer's instructions. After washing with PBS/T, 50 μl of a 1:1000 dilution of anti-human kappa light chain antibody alkaline phosphatase conjugate (Sigma-Aldrich) or anti-DIG-Fab alkaline phosphatase conjugate (Roche Diagnostics) was applied to each well and incubated for 1 h. After final washing with PBS, 50 μl of 0.5 mg/ml p-nitrophenyl phosphate in AP buffer (100 mM Tris/HCl pH 8.8, 100 mM NaCl, 5 mM MgCl₂) was added and signal development was recorded at 405 nm for 15 min at 1 min intervals using a Synergy 2 photometer (BioTek Instruments, Bad Friedrichshall, Germany). The concentration-dependent signals (ΔA/Δt) were evaluated following a published procedure (Voss & Skerra, 1997) using the formula:

[MAb·Ag]=[MAb]ₜ·[Ag]ₜ/(K_{D}+[Ag]ₜ)

[MAb·Ag] is the detectable amount of antibody/antigen complex, which is proportional to the ΔA/Δt signal measured for each well; [MAb]ₜ is the total amount of immobilized antibody, which corresponds to the asymptotic maximal signal of the binding curve; [Ag]ₜ is the (variable) total concentration of PAS antigen applied to each well and K_{D} is the dissociation constant of the antibody/antigen complex resulting from the curve fit, which was evaluated with KaleidaGraph (Synergy Software, Reading, PA).

SPR measurements were performed at 25° C. either on a Biacore X 100 or Biacore T 200 instrument (GE Healthcare) using a mouse antibody capture kit and CM3 sensor chips (both from GE Healthcare). Culture supernatants were diluted 1:5 in HBS-ET buffer (0.01 M HEPES/NaOH pH 7.4, 0.15 M NaCl, 3 mM EDTA, 0.005% v/v Tween20), and a 30 μl sample was injected at a flow rate of 10 μl/min. A concentration series of the following test antigens, as appropriate, was injected onto the sensor ship using single cycle kinetics (Karlsson et al., 2006) at a flow rate of 30 μl/min: PAS #1(200)-IL1Ra (SEQ ID NO: 72), P/A #1(200)-IL1Ra (SEQ ID NO: 73), P/A #1(600)-GMCSF (SEQ ID NO: 75), APSA(200)-IL1Ra (SEQ ID NO: 74) and hu4D5-P/A #1(200) WO 2011/144756 A1. The sensor chip was regenerated with 10 mM glycine/HCl pH 1.7 for 100 s. After subtraction of signals from a reference channel and a blank baseline measured with HBS-ET buffer, data were fitted using the Biacore X100 evaluation software ver. 2.0.1 (GE Healthcare) and a bivalent analyte model. The rate equations used by the fitting algorithm are as follows:

$$Bivalent analyte ( A ) binds to ligand ( B ) .$$

$$A ( solution ) = Conc$$

$$A [ 0 ] = 0$$

$$dA / dt = ( tc * f \hat{} ( 1 / 3 ) ) * ( Conc - A ) - ( ( 2 * ka 1 ) * A * B - kd 1 * AB )$$

$$B [ 0 ] = RMax$$

$$dB / dt = - ( ( 2 * ka 1 ) * A * B - kd 1 * AB ) - ( ka 2 * AB * B - ( 2 * kd 2 ) * AB 2 )$$

$$AB [ 0 ] = 0$$

$$dAB / dt = ( ( 2 * ka 1 ) * A * B - kd 1 * AB ) - ( ka 2 * AB * B - ( 2 * kd 2 ) * AB 2 )$$

$$AB 2 [ 0 ] = 0$$

$$dAB 2 / dt = ( ka 2 * AB * B - ( 2 * kd 2 ) * AB 2 )$$

$$Total response :$$

$$AB + AB 2 + RI$$

Parameters: Conc, analyte concentration [M]; tc, mass transfer constant; f, volume flow rate of solution through the flow cell [m³·s⁻¹]; RMax, binding capacity; RI, refractive index.

D. Cloning of V-Genes from Hybridoma Cells

Hybridoma cells were mechanically lysed and total RNA was extracted using the RNeasy Mini Kit (Qiagen, Hilden, Germany), followed by cDNA synthesis using the First Strand cDNA Synthesis Kit (Thermo Fisher Scientific) with an oligo(dT)₁₈ primer. Ig V-gene regions were PCR-amplified from this cDNA with Q5 DNA polymerase (New England Biolabs, Frankfurt/M. Germany) using a set of 63 forward primers covering all mouse germline V_{L}/V_{H} gene segments (Chardes et al., 1999) together with the reverse primers RMK (5′-GAC CTC CAC GGA GTC AGC-3′; SEQ ID NO: 77) for the light chain and RMG (5′-AGG TCG CCA CAC GTG TGG-3′; SEQ ID NO: 78) for the heavy chain (Loers et al., 2014). Forward primers were initially applied in pools of 5-15 in order to reduce the required number of PCR reactions and, after a PCR product was identified for such a pool, individually to generate a single PCR product. After that, suitable PCR products were isolated by agarose gel electrophoresis using the Wizard SV Gel and PCR Clean-Up System (Promega, Madison, WI) and subjected to double-stranded DNA sequencing using the Mix2Seq Kit (Eurofins Genomics, Ebersberg, Germany).

### E. Construction of Bacterial Expression Plasmids for Fab Fragments

For cloning of the V-genes on the bacterial expression vector pASK88 (Schiweck & Skerra, 1995), the products from the V-gene amplification described above were PCR-amplified with primer pairs that were designed to introduce suitable flanking restriction sites following a previously published routine procedure (Loers et al., 2014; Peplau et al., 2020). The resulting PCR products were cut with the corresponding restriction enzymes, isolated by agarose gel electrophoresis, and the V_{H} and V_{L} genes, respectively, were inserted into pASK88, which had been cut with the corresponding restriction enzymes, in two consecutive ligations. The coding regions for the Anti-PA(S) MAb 2.1, Anti-PA(S) MAb 1.2 and Anti-PA(S) MAb 3.1 were obtained by gene synthesis with suitable flanking restriction sites (GeneArt, Regensburg, Germany) based on V-gene sequences determined for these hybridomas by ProMab Biotechnologies.

F. E. coli Production and Purification of Fab Fragments

pASK88 derivatives harboring the V-genes of Anti-PA(S) MAb 2.1, Anti-PA(S) MAb 2.2, Anti-PA(S) MAb 1.1, Anti-PA(S) MAb 1.2, Anti-PA(S) MAb 3.1 and Anti-PA(S) MAb 3.2 were used to express the chimeric Fab fragments (murine variable domains from the hybridomas fused to human constant domains) either in 6×2 l shake flask culture using E. coli strain JM83 (Yanisch-Perron et al., 1985) or via 8 l bench top fermentation using the strain KS272 (Meerman & Georgiou, 1994) and following published procedures (Schiweck & Skerra, 1995; Skerra, 1994). The recombinant proteins were purified from the periplasmic cell extract via immobilized metal ion affinity chromatography (IMAC), followed by cation exchange chromatography (CEX) on a Resource S 6 ml column and size exclusion chromatography (SEC) on a HiLoad 16/60 Superdex75 prep grade column (both from GE Healthcare). Protein concentrations were determined by measuring the absorbance at 280 nm using calculated extinction coefficients (Gasteiger et al., 2003) of 88405 M⁻¹ cm⁻¹, 89895 M⁻¹ cm⁻¹, 77405 M⁻¹ cm⁻¹, 66405 M⁻¹ cm⁻¹, 69955 M⁻¹ cm⁻¹ or 57465 M⁻¹ cm⁻¹ for the chimeric Fab fragments of Anti-PA(S) MAb 2.1, Anti-PA(S) MAb 2.2, Anti-PA(S) MAb 1.1, Anti-PA(S) MAb 1.2, Anti-PA(S) MAb 3.1 or Anti-PA(S) MAb 3.2, respectively. Protein integrity and purity were checked by SDS-PAGE (Fling & Gregerson, 1986) and electrospray ionization mass spectrometry (ESI-MS) on a maXis Q-TOF instrument (Bruker Daltonics, Bremen).

### G. Antigen Affinity Measurement of Fabs by ELISA, Fluorescence Titration and SPR

A NUNC Maxisorp F 96-well plate was coated with either 50 μl of 10 μg/ml P/A #1(600) polypeptide (Breibeck & Skerra, 2018) in PBS for the recombinant Fab fragments of Anti-PA(S) MAb 2.1 and Anti-PA(S) MAb 2.2, 50 μl of 10 μg/ml PAS #1(600)-Ieptin (Morath et al., 2015) in PBS for the Fab fragments of Anti-PA(S) MAb 1.1 and Anti-PA(S) MAb 1.2, or 50 μl of 10 μg/ml APSA(200)-IL1Ra (SEQ ID NO: 74) for the Fab fragments of Anti-PA(S) MAb 3.1 and Anti-PA(S) MAb 3.2, and incubated at 4° C. overnight. After a single washing step with PBS/T, the wells were blocked with 3% w/v BSA (NeoFROXX, Einhausen, Germany) in PBS/T for 1 h, followed by washing and 1 h incubation with 50 μl of an appropriate dilution series of each purified Fab fragment in PBS/T. The wells were washed again with PBS/T followed by incubation with 50 μl of a 1:1000 dilution of anti-human kappa light chain goat antibody conjugated to alkaline phosphatase (Sigma-Aldrich) in PBS/T for 1 h. After final washing twice each with PBS/T and PBS, signals were developed with p-nitrophenyl phosphate and measured and evaluated as described herein above.

Fluorescence titration was performed as previously described (Voss & Skerra, 1997) using a LS-50B luminescence spectrometer (Perkin Elmer, Norwalk, CT) equipped with a 2 ml quartz cuvette thermostated at 25° C. with wavelengths of 280 nm for excitation and 340 nm for detection (integrating the signal over 5 s). 2 ml of a 1 μM solution of the purified Fab fragment of the Anti-PA(S) MAb 2.2 in 100 mM Tris/HCl pH 7.5 was titrated with a 5 mM solution of the Abz-APAPAAPA peptide (Peptide Specialty Laboratories-PSL, Heidelberg, Germany) (Abz means ortho-aminobenzoyl) in aliquots of 1 μl up to a total volume of 22 μl. Data were normalized to an initial fluorescence of 100% and fitted by non-linear least-squares regression with KaleidaGraph (Synergy Software, Reading, PA) as described (Edwardraja et al., 2017) including correction of the inner filter effect by titration of N-acetyl-tryptophanamide with the same peptide.

SPR measurements with the Fab fragments of the corresponding Anti-PA(S) MAbs were performed at 25° C. on a Biacore X 100 instrument (GE Healthcare). PAS #1(200)-IL1Ra, P/A #1(200)-IL1Ra or thioredoxinA-APSA(200) were biotinylated with a 20-fold molar amount of succinimidyl-6-(biotinamido)hexaonate (Sigma Aldrich) according to the manufacturer's instructions and individually immobilized as ligands on a biotin CAPture chip (GE Healthcare) following the manufacturer's protocol. Before immobilisation of each ligand, the sensorchip was regenerated with two consecutive injections of 30% v/v acetonitrile, 0.25 M NaOH for 120 s as well as 6 M guanidine/HCl, 0.25 M NaOH for 120 s. A concentration series of the recombinant Fab fragment was injected onto the sensorchip using single cycle kinetics and a flow rate of 30 μl/min. After subtraction of signals from both a reference channel and a blank baseline measured with HBS-ET buffer, data were fitted using the Biacore X100 evaluation software ver. 2.0.1 (GE Healthcare) with a 1:1 binding model. The rate equations used by the fitting algorithm are as follows:

$$A ( solution ) = Conc$$

$$A [ 0 ] = 0$$

$$dA / dt = ( tc * f \hat{} ( 1 / 3 ) ) * ( Conc - A ) - ( ka * A * B - kd * AB )$$

$$B [ 0 ] = RMax$$

$$dB / dt = - ( ka * A * B - kd * AB )$$

$$AB [ 0 ] = 0$$

$$dAB / dt = ( ka * A * B - kd * AB )$$

$$Total response :$$

$$AB + RI$$

Parameters: Conc, analyte concentration [M]; tc, mass transfer constant; f, volume flow rate of solution through the flow cell [m³·s⁻¹]; RMax, binding capacity; RI, refractive index.

H. SPOT Synthesis of Immobilized Peptide Arrays and Epitope Mapping Arrays of 20 overlapping 12mer peptides covering the entire amino acid sequence of the PAS #1 or P/A #1 amino acid sequence repeat, or a 10mer peptide comprising the sequence AAPSAAPSAA, consecutively substituted to all twenty proteinogenic amino acids at positions 3 to 8, were synthesized on a hydrophilic membrane according to a standard protocol (Frank, 2002) using a MultiPep SPOT synthesizer (Intavis, Köln, Germany). Detection of binding activity on the membranes was performed according to a published procedure (Zander et al., 2007) after incubating with either the purified Fab fragment or the hybridoma cell culture supernatant containing the secreted MAb, followed by anti-human kappa light chain antibody alkaline phosphatase conjugate (Sigma-Aldrich) or anti-mouse IgG Fc specific antibody alkaline phosphatase conjugate (Sigma-Aldrich), respectively.

### I. Detection of PASylated Proteins by Western Blotting

Anti-PA(S) MAbs from hybridoma supernatants were tested for detection of PASylated proteins on western blots. A set of different PASylated proteins (PAS #1(200)-IL1Ra (SEQ ID NO: 72), P/A #1(200)-IL1Ra (SEQ ID NO: 73), APSA(200)-IL1Ra (SEQ ID NO: 74) as well as, for control, human serum (human serum (PL), pooled; SEQENS IVD/H2B, Limoges, France) diluted 1:200 in water and spiked with 1 μg IL1Ra (Kineret/Anakinra; Swedish Orphan Biovitrum, Stockholm, Sweden) and E. coli BL21 whole cell lysate were subjected to SDS-PAGE followed by semi-dry electrotransfer on a nitrocellulose membrane. After washing with PBS/T, the membrane was incubated with a 1:2000 dilution in PBS/T of anti-PAS MAbs as hybridoma supernatants or a 1:200000 dilution in case of the purified Anti-PA(S) MAb 2.1. Bound MAbs were detected using a 1:50.000 dilution of an anti-mouse IgG Fc-specific goat antibody conjugated with alkaline phosphatase (Sigma-Aldrich) in PBS/T followed by chromogenic reaction with 5-bromo-4-chloro-3-indolyl-phosphate (BCIP) and nitro blue tetrazolium (NBT) (both from Carl Roth, Karlsruhe).

### J. Pharmacokinetic Analysis in Rats

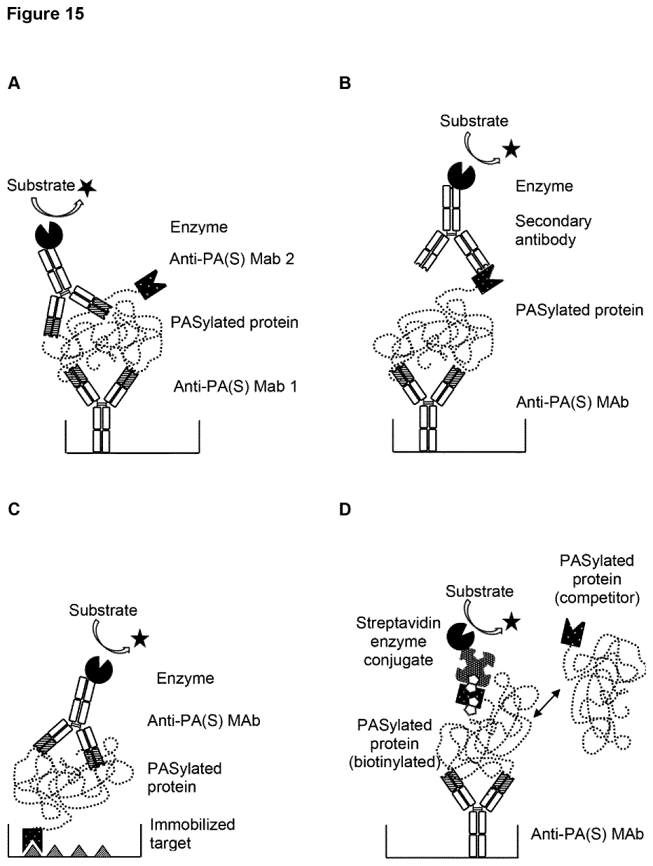

A pharmacokinetic (PK) study in female Wistar rats, at 8-9 weeks age, was conducted at the Aurigon Toxicological Research Center (ATRC, Dunakeszi, Hungary) in compliance with applicable animal welfare regulations. Up to 3 animals per cage were housed in a controlled environment at 22±3° C. with a relative humidity of 50±20%, 12 h light and 12 h dark. Purified PASylated Thymosin alpha 1 (SEQ ID NO. 76) (3.4 mg/kg) was administered subcutaneously via a single injection into the rat dorsal area. Blood samples (100 μl) were taken from 5 animals each at various time points. Following collection in K3-EDTA tubes (Greiner Bio-One, Frickenhausen, Germany), samples were centrifuged at room temperature for 10 min (3000×g) and the resulting plasma was stored at −15 to −30° C. PASylated Thymosin alpha 1 in these samples was quantified using a sandwich ELISA (see Method K & FIG. 15A). Potential alternative ELISA setups suitable for the quantification of PASylated peptides or proteins in blood or plasma/serum samples of animals or human patients are illustrated in FIG. 15, panels B to D.

### K. Quantification of PASylated Thymosin Alpha 1 in Rat Plasma by ELISA

Female Wistar rats (n=5), at 8-9 week age (Aurigon Toxicological Research Center, Dunakeszi, Hungary) were subcutaneously injected with PASylated Thymosin alpha 1 (SEQ ID NO. 76) (3.4 mg/kg) and blood samples (100 μl) were collected in K3-EDTA tubes (Greiner Bio-One, Frickenhausen, Germany) at various time points. For the quantification of PASylated Thymosin alpha 1 administered in the rat PK study (Method J) Nunc Maxisorb ELISA 96 well plates (Thermo Fisher Scientific) were coated with 100 μg/ml of the Anti-PA(S) MAb 2.1 in PBS at 4° C. overnight. After washing twice with PBS/T, free binding sites were blocked with 3% w/v BSA in PBS/T at room temperature for 1 h.

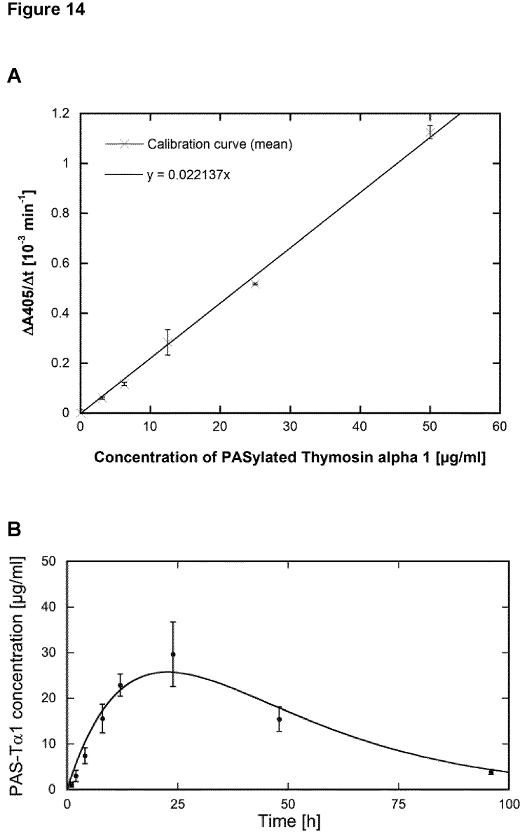

Then, the plate was washed 3 times with PBS/T and the rat plasma samples were applied, each in a 1:2 dilution series, in PBS/T, which had been supplemented with 0.5% (v/v) plasma from an untreated animal in order to maintain a constant proportion of rat plasma constituents. In the same manner, a standard curve was prepared using dilution series of the purified PASylated Thymosin alpha 1 at defined concentrations in PBS/T containing the same amount of rat plasma as the test samples. After incubation for 1 h at room temperature, wells were washed 3 times with PBS/T. To detect bound PASylated Thymosin alpha 1, wells were incubated for 1 h with 50 μl of a 1 μg/ml PBS/T solution of the Anti-PA(S) MAb 1.2, which had been conjugated with alkaline phosphatase using the Lightning-Link alkaline phosphatase antibody labeling kit (BioTechne, Wiesbaden, Germany). After washing twice with PBS/T and twice with PBS, the enzymatic activity was detected using p-nitrophenyl phosphate (0.5 mg/ml). To this end, the plate was incubated for 20 min at 30° C., the absorbance was measured at 405 nm using a SpectraMax M5e microtiter plate reader (Molecular Devices, Sunnyvale, CA), and the PASylated Thymosin alpha 1 concentrations were quantified by comparison with the standard curve (FIG. 14A).

Data were plotted against the sampling time post injection and fitted using a one-compartment model using Phoenix WinNonlin 6.3 software. The resulting PK parameters (Table 1) and PK profile (FIG. 14B) are typical for a long-acting peptide drug and prove that the anti-PAS antibodies of this invention are suited to quantify PASylated drug concentrations in mammalian plasma. Of note, the ELISA setup applied in this method (FIG. 15A) uses an anti-PAS antibody as capture antibody and, thus, avoids the detection of endogenous rat Thymosin alpha 1, which shares 100% sequence identity with the human peptide. Alternative ELISA setups are illustrated, for example, in FIG. 15 and are well known in the art (Vashist & Luong, 2018).

TABLE 1
Pharmacokinetic parameters of PASylated Thymosin alpha 1
(Tα1) in rats. Listed are the maximum serum concentration
of the drug (Cmax), the time to reach Cmax (Tmax), the area under the
curve (AUC), the distribution half-life (t_{1/2}α), the elimination
half-life (t_{1/2}β) and clearance (CL).

| Parameter | PASylated Tα1 |
|---|---|
| Cmax (mg/l) | 25.6 ± 4.4 |
| tmax (h) | 22.7 ± 1.1 |
| AUC_{0-∞} (h μg/ml) | 1586.7 ± 295.1 |
| t_{1/2}α (h) | 15.7 ± 0.8 |
| t_{1/2}β (h) | 15.9 ± 0.9 |
| CL (ml/h/kg) | 2.2 ± 0.4 |

L. Co-Crystallization of Anti-PAS Fab Fragments with PAS Peptides, X-Ray Data Collection and Molecular Model Building

The purified recombinant Fab fragments of Anti-PA(S) MAb 2.2, Anti-PA(S) MAb 1.1 and Anti-PA(S) MAb 3.1 were directly co-crystallized with their cognate PAS peptides, whereas in the case of the Fab of Anti-PA(S) MAb 1.2 a complex with an anti-human kappa V_{H}H domain described in (Ereno-Orbea et al., 2018) was initially prepared. To this end, the purified Fab was incubated for 1 h at 4° C. with a three-fold molar amount of the V_{H}H domain (Thermo Fisher Scientific). The protein mixture was subjected to SEC on a HiLoad 16/60 Superdex75 prep grade column and the Fab·V_{H}H complex was separated from excess anti-human kappa V_{H}H domain and isolated in one peak using 10 mM HEPES/NaOH pH 6.5, 70 mM NaCl as running buffer.

The different protein solutions were concentrated using Amicon Ultracel centrifugal filter units (MWCO 10 kDa; Millipore, Billerica, MA) as follows: Anti-PA(S) MAb 2.2 to 9.6 mg/ml in 20 mM HEPES/NaOH pH 6.5, 80 mM NaCl; Anti-PA(S) MAb 3.1 to 9.2 mg/ml in 10 mM HEPES, pH 6.5, 100 mM NaCl; Anti-PA(S) MAb 1.1 to 8.4 mg/ml and Anti-PA(S) MAb 1.2, as Fab·V_{H}H, to 13.7 mg/ml, both in 10 mM HEPES/NaOH pH 6.5, 70 mM NaCl. For co-crystallization, each concentrated protein solution was mixed with the appropriate peptide from a >50 mM stock solution in water at a molar ratio of 1:3 (Fab:peptide) and incubated for 1 h at 4° C. Then, protein crystallization screens were performed via the sitting drop vapor diffusion method and equivolume mixtures of protein and reservoir solutions, leading to a total drop volume in the range of 300-1000 nl. For refinement of promising crystallization conditions, further screens were set up using the hanging drop vapor diffusion method with a reservoir volume of 1 ml and droplets composed of 1 μl protein and 1 μl reservoir solution. Crystals appeared within one week at 20° C. under the conditions listed in Table 3. Protein crystals were harvested, transferred into the precipitant buffer supplemented with 20% w/v PEG200 for Anti-PA(S) MAb 2.2, 20% w/v ethyleneglycol for Anti-PA(S) MAb 1.1 and Anti-PA(S) MAb 1.2 or 20% w/v glycerol for Anti-PA(S) MAb 3.1 and immediately frozen in liquid nitrogen.

A single-wavelength X-ray synchrotron data set was collected at 100 K from each crystal at the MX beamline BL14.2 of BESSY II operated by the Helmholtz-Zentrum Berlin, Germany or, for the Fab fragment of Anti-PA(S) MAb 3.1, at the protein crystallography beamline X06SA-PXI of the Swiss Light Source (SLS), Villigen-PSI, Switzerland. The diffraction data (Table 3) were reduced with the XDS program package (Kabsch, 2010) and molecular replacement was carried out with Phaser (McCoy et al., 2007) using the constant and variable domains of the Fab 101F (PDB ID: 3QQ9) as search models to solve the structure of the Anti-PA(S) MAb 2.2 Fab·P/A #1 complex. The structures of Anti-PA(S) MAb 1.1 Fab·PAS #1 and Anti-PA(S) MAb 1.2 Fab·PAS #1 were solved by molecular replacement with the refined structure of the Fab of Anti-PA(S) MAb 2.2 as search model, also including the anti-human kappa V_{H}H domain (PDB ID: 6ANA) in the latter case. Structure of anti-PA(S) MAb 3.1 Fab·APSA was solved by molecular replacement with the refined structure of the anti-PA(S) MAb 1.2 Fab as search model, not including the anti-human kappa V_{H}H domain. The protein model was manually adjusted with Coot (Emsley et al., 2010) and refined with Refmac5 (Murshudov et al., 2011). The peptide and water molecules were manually built in Coot in the course of the refinement process. The final structural models were validated using the MolProbity server (Williams et al., 2018). Crystal contact sites as well as accessible and buried surface areas (ASA and BSA, respectively) were analysed with PISA (Krissinel & Henrick, 2007) (calculated with the light and heavy chains connected as a continuous uninterrupted amino acid chain in the input file). Molecular graphics were prepared with PyMOL (Schrödinger, Cambridge, MA) using the APBS module (Baker et al., 2001) for calculation of electrostatics. Atomic distances were calculated with CONTACT (Winn et al., 2011).

Polypeptides were denoted L for the Ig light chain, H for the Ig heavy chain and P for each bound PAS peptide whereas the anti-human kappa V_{H}H domain was assigned the chain identifier X. In case of Anti-PA(S) MAb 1.1, with two Fab·peptide complexes in the asymmetric unit, the one with the higher average crystallographic B-factor was assigned chain identifiers A, B and Q, respectively.

### M. Affinity Purification of StrepII-eGFP-PAS #1(200), H1GA-PAS #1(200)-His₆ and PAS #1(800)-IL1Ra Using an Anti-PAS Fab Immobilized on a Sepharose Column

In total 5 mg of the purified Fab fragment of Anti-PA(S) MAb 1.2 was covalently immobilized on a 1 ml HiTrap NHS-activated HP column (GE Healthcare) according to the manufacturer's protocol. In brief, the column was washed with ice-cold 1 mM HCl prior to injection of the Fab in 1 ml coupling buffer (0.2 M NaHCO₃, 0.5 M NaCl, pH 8.3) and incubation for 30 min at 25° C. Washing and deactivation of excess reactive groups was performed by repeated alternating injections of 0.5 M ethanolamine, 0.5 M NaCl, pH 8.3 and 0.1 M Na-acetate, 0.5 M NaCl, pH 4.

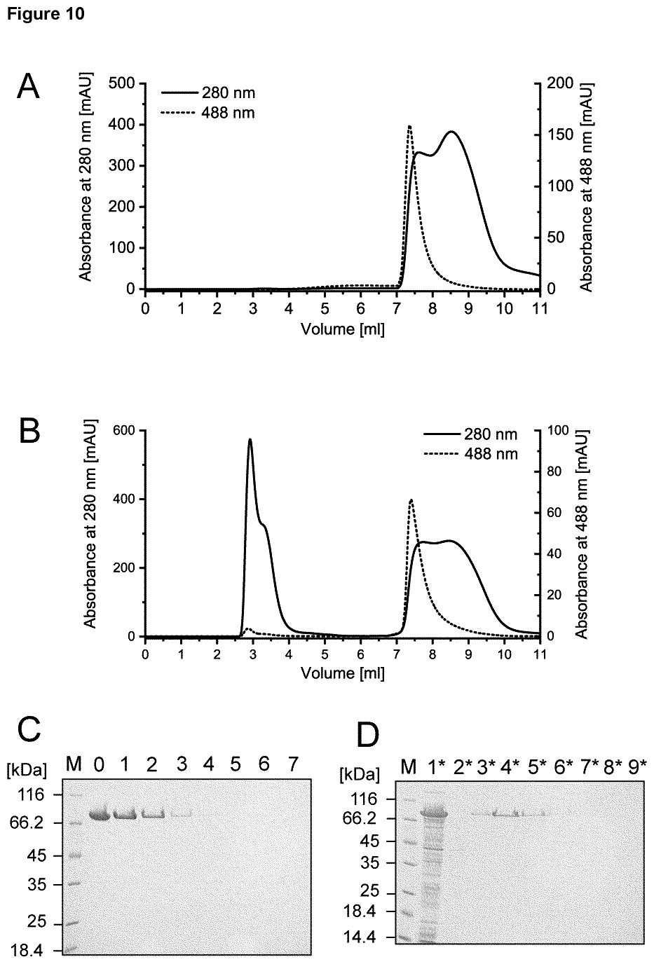

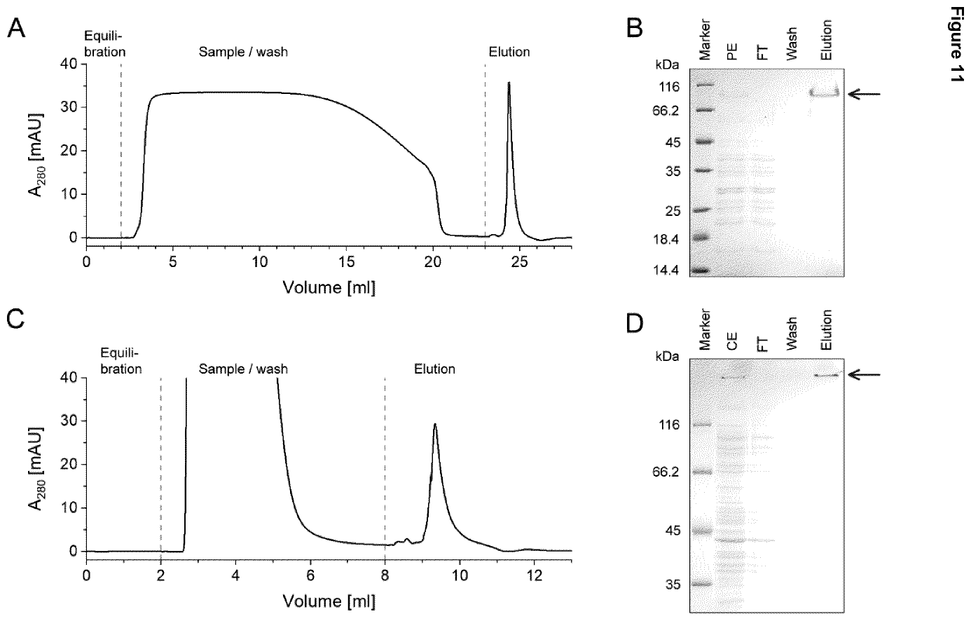

Purification of the PASylated test proteins StrepII-eGFP-PAS #1(200), H1GA-PAS #1(200)-His₆ and PAS #1(800)-IL1Ra on this column was performed using an AKTA Pure 25 chromatography system operated at a flow rate of 1 ml/min. The column was first equilibrated with 2 ml of running buffer (100 mM Tris/HCl pH 8, 150 mM NaCl, 1 mM EDTA), followed by injection of either (i) pure StrepII-eGFP-PAS #1(200) (SEQ ID NO: 71) or (ii) a whole cell lysate of E. coli BL21 cells expressing StrepII-eGFP-PAS #1(200) or (iii) a periplasmic extract of E. coli BL21 cells expressing H1GA-PAS #1(200)-His₆ (SEQ ID NO: 90), or (iv) a whole cell lysate of E. coli BL21 cells expressing PAS #1(800)-IL1Ra (SEQ ID NO: 91). Unbound proteins were washed off the column with 2 ml running buffer, then bound protein was eluted by applying 2-3 ml of a 1 M solution of L-prolinamide (Sigma Aldrich) in running buffer or, alternatively, 1 M L-prolinamide, 100 mM Tris, 150 mM NaCl, 1 mM EDTA, pH adjusted to 8.0 with HCl, followed by regeneration of the column with running buffer. In order to monitor both the presence of proteins in general and the specific presence of StrepII-eGFP-PAS #1(200), UV absorbance was detected at 280 nm and 488 nm, respectively (FIG. 10A, B). SDS-PAGE analysis confirmed the specific elution of the pure PASylated protein (FIG. 10C, D). Due to an apparent impurity in the commercial L-prolinamide substance that led to a background absorption at 280 nm, a blank chromatogram (without application of a PASylated protein) was recorded and used for subtraction to obtain the corrected chromatogram for H1GA-PAS #1(200)-His₆ and PAS #1(800)-IL1Ra (FIGS. 11A, C). Specific elution of the pure PASylated proteins was confirmed by SDS-PAGE analysis (FIGS. 11B, D).

### N. Preparation of PASylated Test Proteins

All test proteins and peptides fused to PAS sequences with different compositions and lengths used in the methods herein described were produced in E. coli either via cytoplasmic expression or via periplasmic secretion from conventional expression vectors harbouring corresponding synthetic genes according to routine procedures well described in the art, e.g. in WO 2008/155134 A1, WO 2011/144756 A1, WO 2017/109087 A1, WO 2018/234455 A1 or in (Binder & Skerra, 2017; Breibeck & Skerra, 2018; Morath et al., 2015; Schlapschy et al., 2013).

### O. Deposits

The following MAbs of this invention were deposited by XL-protein GmbH, Lise-Meitner-Strasse 30, 85354 Freising, Germany as cell cultures at the Leibniz Institute DSMZ-German Collection of Microorganisms and Cell Cultures GmbH, Inhoffenstrasse 7B, 38124 Braunschweig, Germany, which was recognized by the World Intellectual Property Organization as an International Depositary Authority according to the Budapest Treaty for the deposit of animal and human cell cultures on 28 Feb. 1991:
Anti-PA(S)Mab 1.1=DSM ACC3365
Anti-PA(S)Mab 2.1=DSM ACC3366
Anti-PA(S)Mab 3.1=DSM ACC3367

### Example 2: Generation of Monoclonal Anti-PAS Antibodies and their Specific Detection of PAS Sequences

Antibodies against three different PAS peptide sequences, PAS #1 (SEQ ID NO: 1), P/A #1 (SEQ ID NO: 2) and APSA (SEQ ID NO: 3), were raised in mice. To this end, the animals were immunized with corresponding synthetic N-terminally protected 40mer peptides as described in Example 1 herein above that were chemically coupled via their C-terminal carboxylate groups to mariculture keyhole limpet hemocyanin (KLH) as a highly immunogenic T-cell dependent carrier antigen/“immunoadjuvant” (Swaminathan et al., 2014). In the case of PAS #1 and P/A #1 the 40mer covered exactly two copies of the designed 20mer sequence repeat (Breibeck & Skerra, 2018; Schlapschy et al., 2013), whereas the “APSA” peptide comprised 10 copies of the 4-residue motif, which may be considered as a kind of simplified Pro/Ala-rich sequence pattern.

These 2×20mer and/or 40mer PAS peptides were designed in order to encompass at least two copies of the corresponding PAS sequence repeat, thus including at least one copy of the junction between two adjacent sequence repeats, which also constitutes a potential epitope in longer recombinant PAS polypeptides. After four to six rounds of immunization as well as a final boost, each with 25-50 μg antigen, spleen cells were isolated from five mice per antigen and fused with Sp2/0 myeloma cells to generate hybridomas. For each immunization campaign, antibodies from 40 hybridoma clones were characterized by ELISA using recombinant fusion proteins comprising the corresponding PAS polypeptides (200-600 residues), with the goal to screen for (i) sequence-specific and context-independent recognition of PAS sequences and (ii) identification of antibodies showing potential cross-reactivity between the different PAS sequences. MAb capture ELISAs with hybridoma culture supernatants were performed, applying the PAS fusion protein in a concentration-dependent fashion, to determine the dissociation constants (K_{D}). Hybridoma culture supernatants of promising candidates were characterized with regard to antigen affinity and binding kinetics by real-time surface plasmon resonance (SPR) spectroscopy. Corresponding methods are described in Example 1.

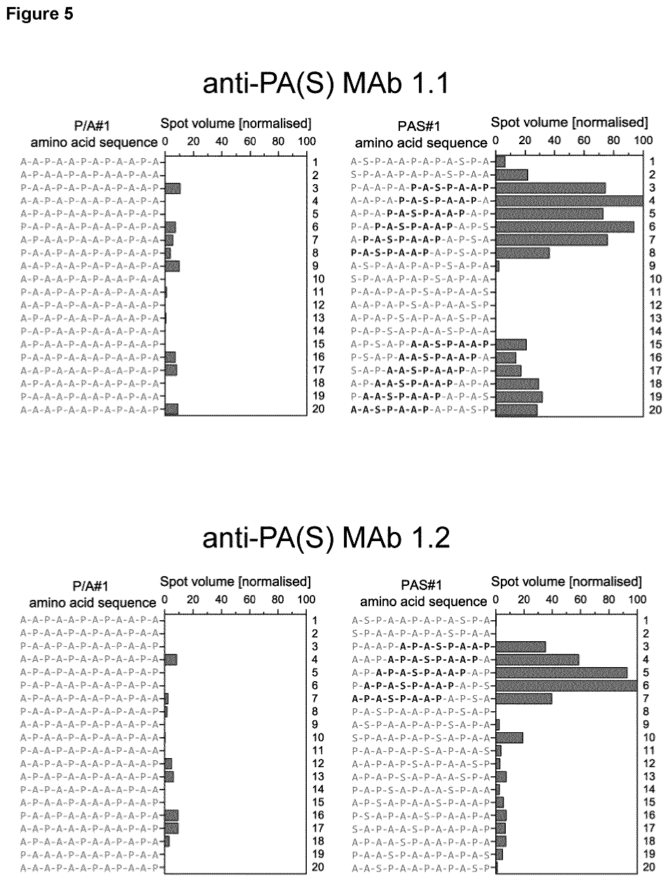

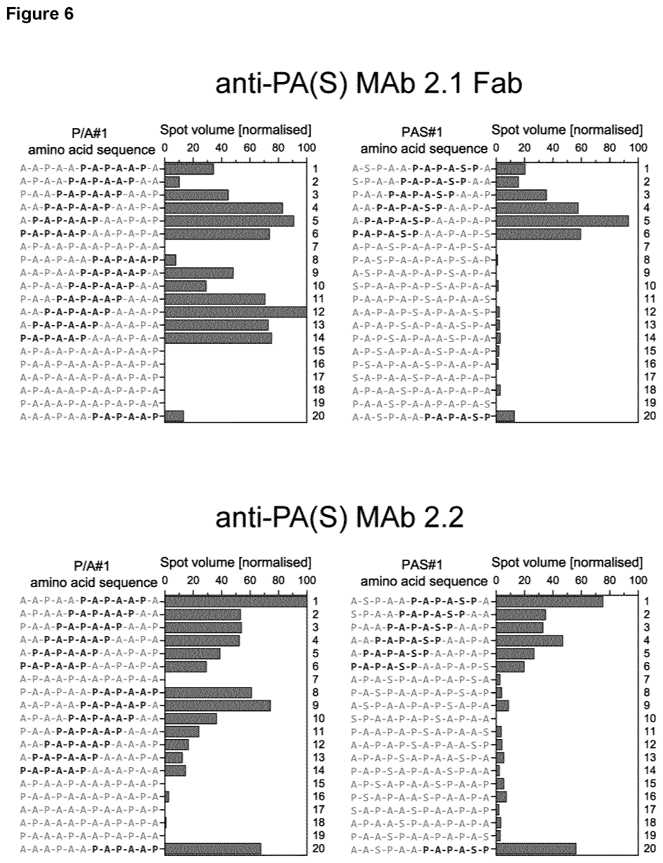

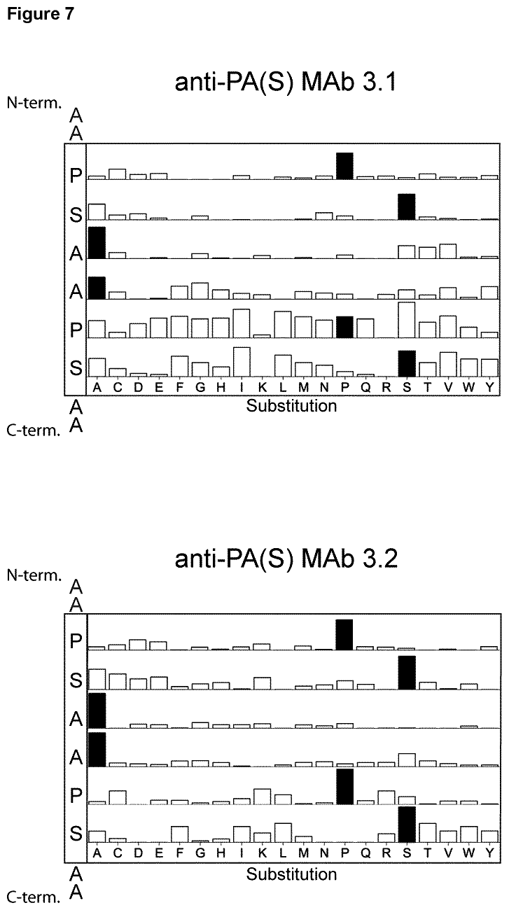

Based on their K_{D} values resulting from the ELISA and SPR measurements, also considering the absorption amplitudes in the concentration-dependent ELISAs, eight clones with distinct properties were selected each from the PAS #1(40)-KLH and P/A #1(40)-KLH immunization and tested for linear epitope recognition on a synthetic peptide array using the Synthetic Peptides On Transfer membranes (SPOT) technique (Frank, 2002). This assay revealed “PAPAAP” (SEQ ID NO: 8) and “PAPASP” (SEQ ID NO: 9) as epitope sequences for the Anti-PA(S) MAbs 2.1 and 2.2, while the Anti-PA(S) MAbs 1.1 and 1.2 predominantly recognized the peptide motif “PASPAAP” (SEQ ID NO: 10) (see FIGS. 5 and 6). Due to the simpler repetitive nature of the APSA sequence, a SPOT substitution analysis was performed for this antigen (FIG. 7). Therefore, a 10-mer peptide comprising the sequence AAPSAAPSAA, consecutively substituted to all twenty essential amino acids at positions 3 to 8, was synthesized C-terminally anchored on a hydrophilic membrane. Interestingly, antibodies generated by immunization with PAS #1(40)-KLH only recognized the PAS #1 polypeptide sequence (Anti-PA(S) MAbs 1.1 and 1.2), while some MAbs from the P/A #1(40)-KLH immunization also showed cross-reactivity with PAS #1 (Anti-PA(S) MAb 2.1) beyond the P/A #1 epitope. Surprisingly, the generalized sequence motif recognized by all Anti-PA(S) MAbs of the invention emerged as “(P/S)A(A/S)P” or as PA(A/S)P.

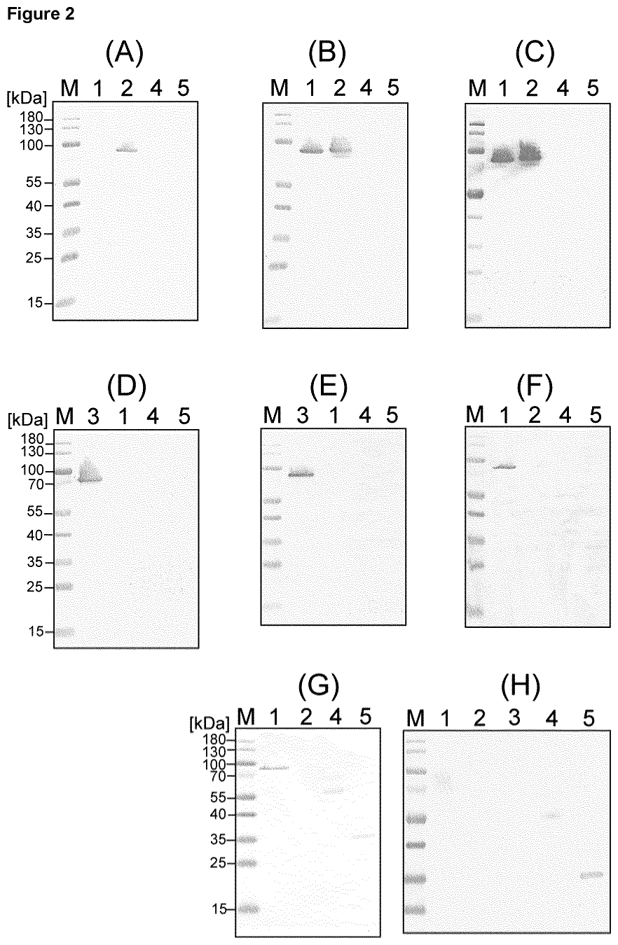

To verify the applicability of the monoclonal antibodies of the invention in the detection of PASylated fusion proteins, the Anti-PA(S) MAbs from the hybridoma supernatants were tested in western blotting experiments wherein specific detection of PASylated fusion proteins was confirmed. Furthermore, no cross-reactivity to the non-PASylated protein version, human serum proteins or proteins in an E. coli whole cell lysate was detected (FIG. 2).

### Example 3: Cloning of V-Gene Sequences of MAbs and Fab Production

For each antigen, the two most promising hybridoma clones were selected for further analysis, based on their affinities to the target sequences as well as cross-reactivity to other PAS sequences: Anti-PA(S) MAb 2.1 and Anti-PA(S) MAb 2.2 for P/A #1; Anti-PA(S) MAb 1.1 and Anti-PA(S) MAb 1.2 for PAS #1, Anti-PA(S) MAb 3.1 and Anti-PA(S) MAb 3.2 for APSA.

To determine their V-gene sequences from the mRNA/cDNA, the coding regions for each V_{H} and V_{L} domain were reverse-transcribed and amplified by polymerase chain reaction (PCR) using suitable oligodeoxynucleotide primers as described in Example 1 herein above. The cloned V-gene sequences (for Anti-PA(S) MAb 2.2, Anti-PA(S) MAb 1.1 and Anti-PA(S) MAb 3.2) or, alternatively, corresponding synthetic DNA fragments (for Anti-PA(S) MAb 2.1, Anti-PA(S) MAb 1.2 and Anti-PA(S) MAb 3.1) were then inserted into a bacterial expression vector encoding the first human IgG1 heavy chain and κ light chain constant regions to allow expression of the corresponding chimeric Fab fragments (Schiweck & Skerra, 1995; Skerra, 1994). The Fab fragments were produced in a functional state by periplasmic secretion in E. coli both at the shake flask and at the bench top fermenter scale and purified to homogeneity by IMAC, CEX and SEC (see Example 1).

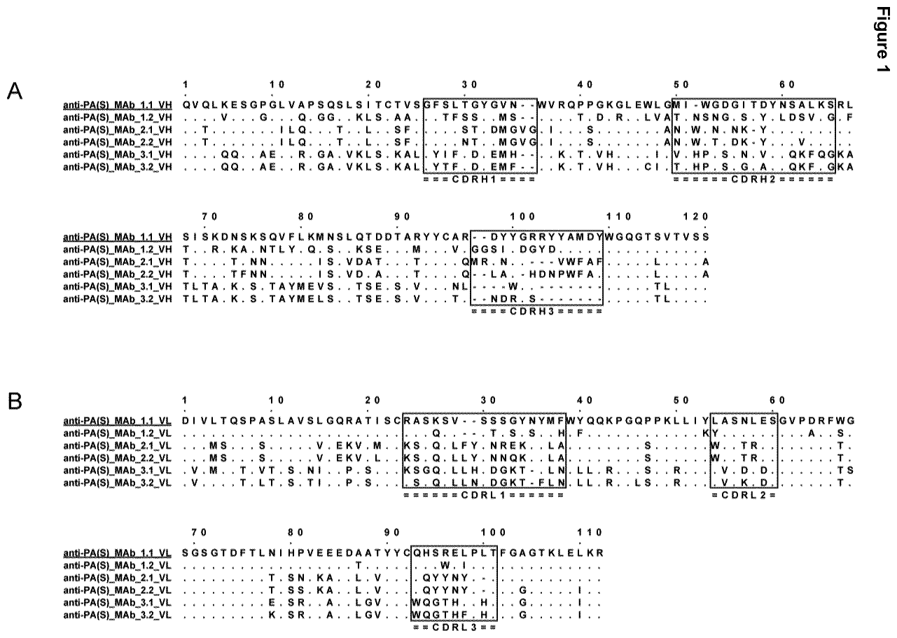

The following amino acid sequences were obtained (see FIG. 1; CDRs according to the definition by (Kabat et al., 1991) are labelled with a black frame):
VH Anti-PA(S) MAb 1.1 (SEQ ID NO: 23):
VL Anti-PA(S) MAb 1.1 (SEQ ID NO: 24):
VH Anti-PA(S) MAb 1.2 (SEQ ID NO: 25):
VL Anti-PA(S) MAb 1.2 (SEQ ID NO: 26
VH Anti-PA(S) MAb 2.1 (SEQ ID NO: 27):
VL Anti-PA(S) MAb 2.1 (SEQ ID NO: 28):
VH Anti-PA(S) MAb 2.2 (SEQ ID NO: 29):
VL Anti-PA(S) MAb 2.2 (SEQ ID NO: 30):
VH Anti-PA(S) MAb 3.1 (SEQ ID NO: 31):
VL Anti-PA(S) MAb 3.1 (SEQ ID NO: 32):
VH Anti-PA(S) MAb 3.2 (SEQ ID NO: 33):
VL Anti-PA(S) MAb 3.2 (SEQ ID NO: 34):

Of note, apart from the method of (Kabat et al., 1991) for determining CDRs, which is largely based on cross-species sequence variability there is at least one other approach well known in the art, which is based on crystallographic studies of antigen-antibody complexes (Al-Lazikani et al., 1997; Chothia et al., 1989). As used herein, a CDR preferentially refers to the definition by Kabat (supra) but may also refer to CDRs defined by the other said approach or by a combination of both approaches. Amino acids were numbered using sequential numbering.

### Example 4: Characterization of Binding Affinities (Anti-PAS Monoclonals and Anti-PAS Fabs

The monoclonal antibodies (MAbs) of the invention and as obtained by the methods and Examples provided herein as well as the corresponding recombinant anti-PAS Fabs were investigated in quantitative ELISAs and real-time SPR measurements in order to precisely determine their K_{D} values towards the different PAS polypeptides (see also Table 2). These measurements essentially confirmed the findings from the preliminary hybridoma screening.

At least one MAb with particularly high affinity was identified for each type of PAS antigen, here evident from a K_{D} value in the one-digit nanomolar range measured for the Fab: 2 nM towards P/A #1 for Anti-PA(S) MAb 2.1; 23 nM towards PAS #1 for Anti-PA(S) MAb 1.1; 2 nM towards APSA for Anti-PA(S) MAb 3.1. Compared with the previously investigated intact MAbs, the affinities measured for the Fabs were usually by 1-2 orders weaker, which is most likely due to the avidity effect that arises when the bivalent MAb interacts with a long PAS polypeptide that harbors multiple copies of the epitope (for example, 30 copies of the repetitive 20 PAS #1 amino acid stretch in a 600-residue PAS polypeptide). Anti-P/A #1 Fabs were either specific for the P/A #1 sequence or cross-reactive with the PAS #1 sequence as well, while the anti-PAS #1 Fabs showed specificity towards the PAS #1 sequence only. Anti-APSA antibody fragments were either specific for the APSA sequence or cross-reactive with the PAS #1 and P/A #1 polypeptides (see the following Table 2).

TABLE 2
Affinities of the MAbs and corresponding Fabs towards their cognate PAS
(poly)peptides measured by ELISA, SPR and fluorescence titration (FT).

| | | | Antigen | | | | | |
|---|---|---|---|---|---|---|---|---|
| | | | PAS#1 | | P/A#1 | | APSA | |
| | | Anti-PA(S) MAb | Hybridoma clone | | | | | |
| Format | Assay | Target | 1.1 | 1.2 | 2.1 | 2.2 | 3.1 | 3.2 |
| MAb | SPR | P/A#1(200)-IL1Ra | n.q. | n.q. | 429 pM | 1.1 nM | n.q. | 20 nM |
| MAb | ELISA | hu4D5- | n.q. | n.q. | 170 pM | 69 nM | n.q. | 400 ± 100 |
| | | P/A#1(200) | | | | | | pM |
| Fab | SPR | P/A#1(200)-IL1Ra | n.d. | n.d. | 648 nM | 7 μM | n.d. | 446 nM |
| Fab | ELISA | P/A#1(600) | n.q. | n.q. | 2 ± 0.2 nM | 8 ± 1 nM | n.q. | 48 ± 4 nM |
| MAb | SPR | PAS#1(200)- | 1 nM | 44 nM | 1.7 nM | 22 nM | n.q. | 80 nM |
| | | IL1Ra | | | | | | |
| MAb | ELISA | hu4D5- | 37 ± 10 | 162 ± 11 | 113 pM | n.q. | n.q. | 294 ± 92 nM |
| | | PAS#1(200) | pM | pM | | | | |
| Fab | SPR | PAS#1(200)- | 8.8 μM | 3.7 μM | 4.4 nM | n.d. | n.d. | n.q. |
| | | IL1Ra | | | | | | |
| Fab | ELISA | PAS#1(600)- | 23 ± 5 | 123 ± 20 | 2.2 ± 0.1 | n.q. | n.q. | 311 ± 16 nM |
| | | Leptin | nM | nM | nM | | | |
| MAb | SPR | APSA(200)-IL1Ra | n.q. | n.q. | n.q. | n.q. | 19 pM | 108 pM |
| MAb | ELISA | APSA(200)-IL1Ra | n.q. | n.q. | n.q. | n.q. | 118 ± 2 pM | 251 ± 5 pM |
| Fab | SPR | APSA(200)-TrxA | n.d. | n.d. | n.d. | n.d. | 2.5 nM | 137 nM |
| Fab | ELISA | APSA(200)-IL1Ra | n.d. | n.d. | n.d. | n.d. | 1.9 ± 0.01 | 1.6 ± 0.1 |
| | | | | | | | nM | nM |
| Fab | FT | Abz-APAPAAPA | n.d. | n.d. | >100 μM | 9.2 ± 0.1 μM | n.d. | n.d. |

n.q.: not quantifiable
n.d.: not determined

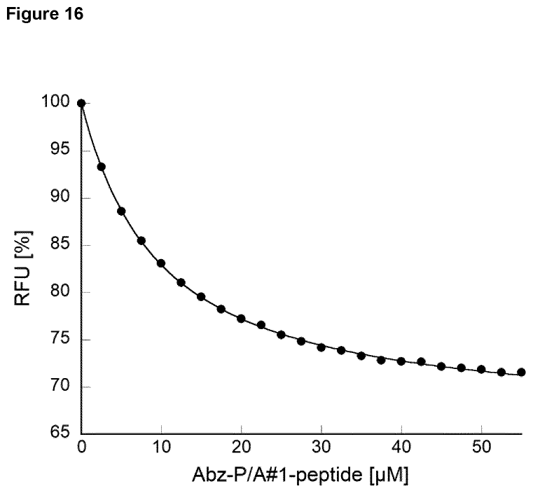

To determine the monovalent affinity of Anti-PA(S) MAb 2.1 and Anti-PA(S) MAb 2.2 towards their epitope sequence, fluorescence titration (FT) experiments were performed with the corresponding recombinant Fab fragments and the synthetic peptide Abz-APAPAAPA (SEQ ID NO: 4) (carrying an N-terminal o-aminobenzoyl group as fluorescence resonance energy transfer probe). While no reliable K_{D} value could be deduced for the Fab of the Anti-PA(S) MAb 2.1, a K_{D}=9.2±0.1 μM was determined for the Fab of the Anti-PA(S) MAb 2.2 (FIG. 16). This value has to be compared with K_{D}=8±1 nM measured for the pure P/A #1(600) antigen in the ELISA, which was ˜1000 fold higher (see Table 2). This difference must even be higher for the Fab of the Anti-PA(S) MAb 2.1 and indicates that not only the avidity effect but also more peculiar aspects, such as the precise molecular neighborhood of the epitope peptide or the epitope density within a polypeptide with many sequence repeats, can considerably influence the apparent affinity.

### Example 5: Structural Characterization of PAS Peptide Binding by Co-Crystallization with Anti-PAS Fabs

The structural mechanism of antigen recognition by some of the Anti-PA(S) MAbs of this invention was analyzed using X-ray crystallography. Accordingly, recombinant anti-PA(S) Fab fragments as prepared using the methods described in Example 1 herein above were subjected to co-crystallization experiments with their cognate synthetic peptides, whose sequences were either based on the epitope sequences determined by the SPOT assay as described above or, in case of the simple APSA motif, comprised a twelve amino acid stretch with three APSA repeats. To avoid charges at the N-termini, which would be absent in longer (poly)peptide stretches, these were blocked with pyroglutamic acid (Pga) or by acetylation. Diffraction quality crystals were obtained for the Fab·peptide complexes of Anti-PA(S) MAb 2.2 Fab·P/A #1 and Anti-PA(S) MAb 1.1 Fab·PAS #1 (see appended Table 3). In case of Anti-PA(S) MAb 2.1 and Anti-PA(S) MAb 1.2 we applied a recently published strategy that utilizes an anti-human kappa light chain V_{H}H domain to facilitate (co)-crystallization of (our chimeric) Fab fragments (Ereno-Orbea et al., 2018). Indeed, this approach led to crystals for the Fab of the Anti-PA(S) MAb 1.2 in complex with the PAS #1 epitope peptide, which diffracted to a high resolution of 1.55 Å at a synchrotron X-ray source. The structure of the Anti-PA(S) MAb 2.2 Fab·P/A #1 complex was solved by molecular replacement using the constant and variable domains of the functionally unrelated anti-human RSV Fab 101F (PDB ID: 3QQ9) as search models. Subsequently, the structures of the complexes Anti-PA(S) MAb 1.1 Fab·PAS #1 and Anti-PA(S) MAb 1.2 Fab·PAS #1·V_{H}H were solved by molecular replacement with the refined structure of the Fab 3F3E2Anti-PA(S) MAb 2.2 as search model, as well as the anti-human kappa light chain V_{H}H domain (PDB ID: 6ANA) in the latter case. The structure of Anti-PA(S) MAb 3.1 Fab·(APSA)₃ was solved by molecular replacement with the refined structure of the Anti-PA(S) MAb 1.2 Fab as search model, not including the anti-human kappa VHH domain in this case. After manual positioning of the PAS #1, P/A #1 and (APSA)₃ peptides, crystallographic refinement was completed, leading to R_{free} values of 23-27% (Table 3).

TABLE 3
X-ray diffraction and refinement statistics for recombinant Fab fragments
of Anti-PAS MAbs crystallized in complex with PAS peptide epitopes.

| | Anti-PA(S) MAb | | Anti-PA(S) MAb | | Anti-PA(S) MAb | | Anti-PA(S) MAb | |
|---|---|---|---|---|---|---|---|---|
| | 2.2 | | 1.1 | | 1.2 + VHH | | 3.1 Fab | |
| Peptide ligand | Ac-APAPAAPA | | Pga-APASPAAPA | | Pga-APASPAAPA | | Pga-APSAAPSAAPSA | |
| Crystallization condition | 11% w/v | | 18% w/v | | 22% w/v | | 20% w/v | |
| | PEG6000 | | PEG3350 | | PEG3350 | | PEG3350 | |
| | 100 mM | | 100 mM HEPES | | 300 mM K- | | 200 mM Li- | |
| | Tris/HCl PH 8 | | pH 7.5 | | acetate | | Nitrate | |
| | 200 mM MgCl₂ | | 200 mM MgCl₂ | | | | | |
| Data collection statistics | | | | | | | | |
| Wavelength (Å) | 0.9184 | | 0.9184 | | 0.9184 | | 1 | |
| Resolution range (Å) | 35-2.55 | (2.65-2.55)* | 35-2.65 | (2.75-2.65) | 35-1.55 | (1.65-1.55) | 30-1.85 | (1.95-1.85) |
| Space group | I422 | | P4₂2₁2 | | C2 | | C2 | |
| Unit cell a, b, c (Å) | 122.0, 122.0, 138.4 | | 102.6, 102.6, 199.9 | | 141.3, 44.4, 93.0 | | 94.9, 61.3, 78.6 | |
| α, β, γ (°) | 90.0, 90.0, 90.0 | | 90.0, 90.0, 90.0 | | 90.0, 109.1, 90.0 | | 90.0, 106.3, 90 | |
| assemblies per asym. unit | 1 | | 2 | | 1 | | 1 | |
| Unique reflections | 17370 | (1866) | 30855 | (3054) | 77314 | (12963) | 36237 | (5351) |
| Multiplicity | 26.2 | (25.2) | 22.2 | (18.3) | 6.9 | (6.8) | 3.0 | (3.1) |
| Completeness (%) | 99.9 | (100) | 96.9 | (93.3) | 97.2 | (95.8) | 97.6 | (99.2) |
| Mean I/σl | 17.0 | (5.1) | 24.3 | (2.1) | 15.0 | (1.6) | 9.2 | (2.1) |
| Wilson B-factor (Å²) | 28.3 | | 63.3 | | 30.7 | | 36.5 | |
| Rₘₑₐₛ (%) | 21.3 | (75.1) | 11.2 | (167.5) | 6.3 | (107.0) | 8.3 | (74.6) |
| Refinement statistics | | | | | | | | |
| Resolution (Å) | 34.63-2.55 | (2.62-2.55) | 34.13-2.65 | (2.72-2.65) | 33.39-1.55 | (1.59-1.55) | 45.6-1.85 | (1.898-1.85) |
| R_{work} (%) | 19.5 | (26.2) | 22.0 | (39.4) | 19.4 | (34.1) | 21.3 | (35.9) |
| R_{free} (%) | 23.8 | (32.0) | 26.9 | (37.8) | 23.2 | (35.4) | 26.8 | (37.8) |

| Number of non-H atoms: | | | | |
|---|---|---|---|---|
| Protein/peptide | 3379/50 | 6662/122 | 4374/61 | 3490/77 |
| Solvent | 125 | 53 | 388 | 176 |
| Average B value (Å²) | | | | |
| Protein/peptide | 29.6/42.8 | 73.8/58.5 | 28.7/25.7 | 44.6/58.7 |
| Solvent | 24.7 | 46.2 | 34.5 | 41.2 |
| RMS (bonds/angles) | 0.004/1.327 | 0.004/1.357 | 0.011/1.685 | 0.009/1.532 |
| Ramachandran statistics^{#} | | | | |
| Favored (%) | 96.8 | 94.1 | 97.7 | 96.6 |
| Outliers (%) | 0 | 0.2 | 0 | 0 |
| Rotamer outliers (%) | 1.5 | 2.9 | 0 | 1 |
| Clash score^{#} | 1.63 | 2.47 | 1.6 | 2.09 |

*Values for the highest resolution shell are shown in brackets.
^{#}Calculated with MolProbity (Williams et al., 2018).

Further analysis of these crystal structures showed that the PAS peptides were bound to all four Fabs in a more or less relaxed conformation, covering a wide area of the antigen-binding site with at least four of the six complementarity-determining regions (CDRs) involved. Due to the lack of polar side chains—except for one Ser residue in the PAS #1 epitope peptide and three Ser residues in the (APSA)₃ peptide—the interactions are predominantly mediated through hydrogen bonds with peptide main-chain atoms (see appended Table 4) and Van-der-Waals contacts (see appended Table 5) including some local hydrophobic interactions, whereas salt bridges are completely absent, as expected. Interestingly, in each case at least one Ala residue of the PAS peptide is involved in relevant interactions with the anti-PAS Fab; hence, Ala can be considered as a hot spot for antibody interactions in PAS epitopes. Up to now, Ala, the amino acid with the smallest side chain, has been regarded to play a negligible role in protein-protein/peptide recognition. In fact, the strategy of alanine-scanning mutagenesis (Cunningham & Wells, 1989) has found wide application to dissect critical residues for receptor-ligand or antibody-antigen binding, assuming a quasi inert role of the Ala methyl side chain for molecular interactions. Unexpectedly, this invention reveals that Ala actually can adopt a central role in antigen recognition, as exemplified in particular with two crystal structures, the Anti-PA(S) MAb 2.2 Fab·P/A #1 and the Anti-PA(S) MAb 1.1 Fab·PAS #1. Indeed, being completely buried in the binding pocket, and with its carbonyl oxygen involved in two hydrogen bonds, Ala^{P5} acts as a “hot spot” residue (Clackson & Wells, 1995) in the antibody-peptide interface of the complex Anti-PA(S) MAb 2.2 Fab·P/A #1. Likewise, the structure of the Anti-PA(S) MAb 1.1 Fab reveals a hole in the middle of the antigen-binding site which is perfectly molded to accommodate the methyl group of Ala^{P7}, thereby allowing high shape complementarity and a densely packed interface.

The structure of anti-PA(S) MAb 3.1 Fab in complex with the (APSA)₃ peptide reveals a distinct groove in the paratope between V_{H} and V_{L} chains in which the peptide is bound in an elongated shape. Binding involves residues from all three APSA repeats in the peptide and is primarily mediated by hydrogen bonds with the peptide main chain atoms or peptide Ser side chains, as well as hydrophobic interactions of peptide Pro and Ala side chains. Similar to the structures of the Anti-PA(S) MAb 2.2 Fab·P/A #1 and the Anti-PA(S) MAb 1.1 Fab·PAS #1, Ala residues in the epitope play an important role in mediating hydrogen bonds and Van-der-Waals contacts (Tables 4 and 5).

Unexpectedly, in the case of the Anti-PA(S) MAb 1.2 Fab in complex with the PAS #1 epitope peptide the N-terminal pyroglutamyl residue of the peptide also contributes to the complex formation with three hydrogen bonds. These hydrogen bonds would not be possible in a complex with a longer PAS #1 (poly)peptide where the position of the Pga residue would be occupied by Pro. While a Pro residue would fit perfectly at this position in the crystal structure, the further N-terminal course of a longer polypeptide chain would lead to a steric clash with the Fab.

In order to elucidate any conformational similarities between the bound PAS #1 peptides in the complexes with the anti-PA(S) MAb 1.1 Fab or anti-PA(S) MAb 1.2 Fab, a superposition between their structures was performed. Indeed, the four residues Ser^{P5} to Ala^{P8} showed an excellent match of their C_{α} positions, with a root mean square deviation (RMSD) of only 0.15 Å. Secondary structure analysis with STRIDE (Frishman & Argos, 1995) identified a type I β-turn for this four-residue stretch. Apart from the intramolecular hydrogen bond between the Ser^{P5} carbonyl oxygen and the Ala^{P8} amide hydrogen, this turn is stabilized by a hydrogen bond between the Ser^{P5} hydroxyl group and the Ala^{P7} amide hydrogen. This type of β-turn is classified as SPXX turn and occurs in gene regulatory proteins where it acts as DNA-binding motif (Suzuki & Yagi, 1991). However, despite their mutual similarity in the two Fab complexes, these turns are bound in different orientations: in the complex of anti-PA(S) MAb 1.1 Fab·PAS #1 this turn nestles into the binding pocket whereas it is exposed to the solvent in the complex with the anti-PA(S) MAb 1.2 Fab·PAS #1. Of note, a similar analysis with STRIDE identified no secondary structure features neither for the P/A #1 epitope peptide in the complex with the anti-P/A #1 MAb 2.2 Fab, nor for the (APSA)₃ peptide in complex with anti-PA(S) MAb 3.1 Fab.

In the context of this invention, MAbs that specifically recognize linear epitopes in structurally disordered Pro/Ala-rich (poly)peptides with three different sequences; i.e. sequences as provided in SEQ ID Nos: 1, 2 and 3 are generated by means and methods as provided herein. The inventive anti-PA(S) MAbs, or their recombinant versions and fragments, offer valuable bioanalytical and diagnostic tools for the biochemical study as well as biopharmaceutical development of PASylated drug candidates (Binder & Skerra, 2017; Gebauer & Skerra, 2018; Richter et al., 2020), including suitable assays for clinical studies.

TABLE 4
Hydrogen-bonding interactions between Anti-
PA(S) MAbs and PAS epitope peptides.

| | Peptide | Fab | Distance |
|---|---|---|---|
| Structure | atom | atom | [Å] |
| Anti-PA(S) MAb | Ace^{P1}-O | Tyr^{L98}-O^{η} | 2.47 |
| 2.2 Fab•P/A#1 | Ala^{P3}-N | Tyr^{L98}-O | 2.85 |
| | Ala^{P5}-O | Asn^{H52}-N^{δ2} | 3.17 |
| | Ala^{P5}-O | Trp^{H54}-N^{ε1} | 2.94 |
| | Pro^{P7}-O | Asp^{H104}-O^{δ2} | 2.67 |
| Anti-PA(S) MAb | Pro^{P3}-O | Tyr^{L36}-O^{η} | 2.61 |
| 1.1 Fab•PAS#1 | Ser^{P5}-N | Arg^{H103}-O | 2.87 |
| | Ser^{P5}-O^{γ} | Ser^{L95}-O | 2.46 |
| | Pro^{P9}-O | Arg^{H103}-N^{η} | 2.42 |
| Anti-PA(S) MAb | Pga^{P1}-N | Asp^{H107}-O | 2.80 |
| 1.2 Fab•PAS#1 | Pga^{P1}-N | Tyr^{L54}-O^{η} | 2.89 |
| | Pga^{P1}-O^{ε} | Ala^{H109}-N | 2.96 |
| | Ala^{P2}-N | Asp^{H107}-O | 3.05 |
| | Ala^{P2}-O | Asp^{H107}-N | 2.79 |
| | Pro^{P3}-O | Tyr^{L36}-O^{η} | 2.59 |
| | Ala^{P4}-N | Gly^{H105}-O | 2.99 |
| | Ser^{P5}-O^{γ} | Trp^{L96}-O | 2.50 |
| | Ala^{P10}-O^{oxt} | Asp^{H104}-N | 3.09 |
| Anti-PA(S) MAb | Pga^{P1}-O | Ile^{H28}-N | 3.05 |
| 3.1 Fab•APSA | Ser^{P4}-O^{γ} | Trp^{H99}-O | 2.69 |
| | Ala^{P6}-O | ArgL51-N^{η} | 2.74 |
| | Ser^{P8}-O^{γ} | ArgL51-N^{η} | 2.95 |
| | Ser^{P8}-O | Asn^{L39}-N^{δ2} | 2.84 |
| | Ala^{P10}-N | Gly^{L96}-O | 3.08 |
| | Ala^{P13}-N | Glu^{H33}-O^{ε2} | 3.04 |

TABLE 5
Van-der-Waals atom contacts between Anti-PAS MAbs
and the PAS epitope peptides (≤4.0 Å).

| | Peptide | Number of | |
|---|---|---|---|
| Structure | residue | contacts | Fab residues |
| Anti-PA(S) MAb | Ace^{P1} | 5 | Tyr^{L98} |
| 2.2 Fab•P/A#1 | Ala^{P1} | 8 | Tyr^{L31}, Tyr^{L98} |
| | Pro^{P2} | 25 | Tyr^{L31}, Tyr^{L38}, Tyr^{L98} |
| | Ala^{P3} | 10 | Tyr^{L98}, Asn^{L99}, Tyr^{L100} |
| | Pro^{P4} | 5 | Tyr^{L97}, Tyr^{L98}, Tyr^{L100}, Pro^{H106} |
| | Ala^{P5} | 18 | Tyr^{L100}, Pro^{L101}, Trp^{H49} Asn^{H52}, Trp^{H54}, Tyr^{H60} |
| | Ala^{P6} | 17 | Trp^{H54}, Tyr^{H60}, Asp^{H104} |
| | Pro^{P7} | 22 | Trp^{H54}, Trp^{H55}, Thr^{H56}, Asp^{H58} Tyr^{H60}, Asp^{H104} |
| | Ala^{P8} | — | — |
| Anti-PA(S) MAb | Pga^{P1} | 4 | Tyr^{L34} |
| 1.1 Fab•PAS#1 | Ala^{P2} | 10 | Tyr^{L34}, Tyr^{H104} |
| | Pro^{P3} | 11 | Ser^{L32}, Tyr^{L34}, Tyr^{L36}, Tyr^{H104} |
| | Ala^{P4} | 7 | Tyr^{L36}, Arg^{H103} |
| | Ser^{P5} | 21 | Tyr^{L36}, Ser^{L95}, Arg^{H103}, Tyr^{H104}, Tyr^{H105} |
| | Pro^{P6} | 7 | Ser^{L31}, Tyr^{L36}, Arg^{L96} |
| | Ala^{P7} | 11 | Ser^{L95}, Arg^{L96}, Glu^{L97}, Leu^{L98}, Tyr^{H105} |
| | Ala^{P8} | — | — |
| | Pro^{P9} | 10 | Trp^{H52}, Ile^{H56}, Arg^{H103} |
| | Ala^{P10} | — | — |
| Anti-PA(S) MAb | Pga^{P1} | 30 | Lys^{L53}, Tyr^{L54}, Tyr^{H106}, Asp^{H107}, Tyr^{H108}, Ala^{H109} |
| 1.2 Fab•PAS#1 | Ala^{P2} | 15 | Tyr^{L34}, Tyr^{L54}, Tyr^{H106}, Asp^{H107} |
| | Pro^{P3} | 13 | Tyr^{L34}, Tyr^{L36}, Gly^{H105} |
| | Ala^{P4} | 13 | Tyr^{L36}, Ile^{H102}, Gly^{H105}, Tyr^{H106}, Asp^{H107} |
| | Ser^{P5} | 16 | Tyr^{L36}, Trp^{L96} |
| | Pro^{P6} | 3 | Thr^{L31}, Trp^{L96} |
| | Ala^{P7} | 7 | Trp^{L96}, Glu^{L97} |
| | Ala^{P8} | 2 | Trp^{L96}, Ile^{H102} |
| | Pro^{P9} | 7 | Ile^{L98}, Ile^{H102}, Tyr^{H103} |
| | Ala^{P10} | 7 | Tyr^{H103}, Asp^{H104} |
| Anti-PA(S) MAb | Pga^{P1} | 9 | Tyr^{H27}, Ile^{H28}, Tyr^{H32} |
| 3.1 Fab•APSA | Ala^{P2} | 9 | Tyr^{H27}, Tyr^{H32} |
| | Pro^{P3} | 22 | Val^{H2}, Tyr^{H27}, Tyr^{H32}, Leu^{H98}, Trp^{H99}, Arg^{H101} |
| | Ser^{P4} | 12 | Arg^{L51}, Trp^{H99}, Arg^{H101} |
| | Ala^{P5} | 3 | Tyr^{L54}, Trp^{H99} |
| | Ala^{P6} | 22 | Arg^{L51}, Tyr^{L54}, Leu^{L55}, Trp^{H99} |
| | Pro^{P7} | 22 | Arg^{L51}, Trp^{H99} |
| | Ser^{P8} | 29 | Tyr^{L37}, Asn^{L39}, Arg^{L51}, Gly^{L96}, Trp^{L94}, Trp^{H99}, Gly^{H100} |
| | Ala^{P9} | 17 | Tyr^{L37}, Gly^{L96}, His^{L101} |
| | Ala^{P10} | 16 | His^{L31}, Tyr^{L37}, Gly^{L96} |
| | Pro^{P11} | 6 | Glu^{H33} |
| | Ser^{P12} | 9 | Leu^{L99}, Glu^{H33}, Val^{H59} |
| | Ala^{P13} | 16 | Glu^{H33}, Ile^{H51}, His^{H52}, Asn^{H57} |

In all crystallized Fab complexes provided herein, a high abundance of Tyr residues in the paratope is evident. These residues are responsible for the majority of hydrophobic contacts (appended Table 5), thus creating a surface well suited to bind antigens poor in charge or polar side chains. In fact, 62%, 49% 43% and 23% of all contacts ≤4.0 Å are mediated by Tyr in the Anti-PA(S) MAb 2.2, Anti-PA(S) MAb 1.2, Anti-PA(S) MAb 1.1 and Anti-PA(S) MAb 3.1, respectively. Interestingly, the Anti-PA(S) MAb 2.2 revealed a high Tyr content and also has a high affinity among the crystallized complexes. This is in line with previous analyses, which indicate that a high content of Tyr in antibody paratopes generally contributes to enhanced antigen specificity and affinity (Birtalan et al., 2008; Birtalan et al., 2010).

The data provided herein shed light on the mechanism of molecular recognition of disordered epitopes by antibodies. With no salt bridges and no pronounced side chain interactions arising from the PAS epitope peptides in all assessed Fab structures, complex formation is mainly driven by hydrogen bonds involving the peptide backbone (appended Table 4) as well as Van-der-Waals contacts (appended Table 5) including some local hydrophobic interactions. Due to the feature-less nature of the PAS peptides, the few atom groups capable of polar interactions have to be capitalized efficiently. This is nicely demonstrated with the structure of Anti-PA(S) MAb 1.2, for example, where a short segment of the backbone hydrogen bond network with the PAS #1 peptide resembles an antiparallel β-sheet. In the two Fab complexes with the PAS #1 epitope peptide, which comprises one Ser residue, both antibodies engage the only available polar side chain for formation of hydrogen bonds.

The same is the case in the structure of Anti-PA(S) MAb 3.1, where two of the three Ser side chains are involved in hydrogen bonding. Nevertheless, in line with the limited energy gain of such hydrogen bonds in a competing aqueous environment (Gao et al., 2009). In fact, it seems that the Anti-PA(S) MAbs 1.1 and 1.2 do not much benefit from this interaction considering their significantly lower affinity compared with the best MAbs raised against anti-P/A #1 (see Table 2). The observation that at least four of the six CDRs are involved in the peptide-antibody interactions in all assessed Fab complexes (see Table 6) highlights the need to involve an extended interface to more or less tightly bind structurally flexible antigens.

TABLE 6
CDRs involved in atom contacts between Anti-PA(S) MAbs and the
PAS epitope peptides (≤4.0 Å). Sequential numbering is used for the
amino acid sequences of all antibodies; hence, the numbers of the
CDRs may be individually different.

| | CDR L1 | CDR L2 | CDR L3 | CDR H1 | CDR H2 | CDR H3 |
|---|---|---|---|---|---|---|
| Anti-PA(S) | L24-38 | L54-60 | L93-101 | H26-35 | H50-65 | H98-109 |
| MAb 1.1 | Ser^{L32} | — | Ser^{L95} | — | Trp^{H52} | Arg^{H103} |
| | Tyr^{L34} | | Arg^{L96} | | Ile^{H56} | Tyr^{H104} |
| | Tyr^{L36} | | Glu^{L97} | | | Tyr^{H105} |
| | | | Leu^{L98} | | | |
| Anti-PA(S) | L24-38 | L54-60 | L93-101 | H26-35 | H50-66 | H99-112 |
| MAb 1.2 | Tyr^{L31} | Lys^{L53} | Trp^{L96} | — | — | Ile^{H102} |
| | Tyr^{L34} | Tyr^{L54} | Glu^{L97} | | | Tyr^{H103} |
| | Tyr^{L36} | | Ile^{L98} | | | Asp^{H104} |
| | | | | | | Gly^{H105} |
| | | | | | | Tyr^{H106} |
| | | | | | | Asp^{H107} |
| | | | | | | Tyr^{H108} |
| | | | | | | Ala^{H109} |
| Anti-PA(S) | L24-40 | L56-62 | L95-102 | H26-37 | H52-67 | H100-110 |
| MAb 2.2 | Tyr^{L31} | — | Tyr^{L97} | — | Asn^{H52} | Asp^{H104} |
| | Tyr^{L38} | | Tyr^{L98} | | Trp^{H54} | Pro^{H106} |
| | | | Asn¹⁹⁹ | | Trp^{H55} | |
| | | | Tyr^{L00} | | Thr^{H56} | |
| | | | Pro^{L101} | | Asp^{H58} | |
| | | | | | Tyr^{H60} | |
| Anti-PA(S) | L24-39 | L55-61 | L94-102 | H26-35 | H50-66 | H99-101 |
| MAb 3.1 | His^{L31} | Leu^{L55} | Gly^{L96} | Tyr^{H27} | Ile^{H51} | Trp^{H99} |
| | Tyr^{L37} | | | Ile^{H28} | His^{H52} | Arg^{H101} |
| | Asn^{L39} | | | Tyr^{H32} | Asn^{H57} | |
| | | | | Glu^{H33} | Val^{H59} | |

### Example 6: SPR Spectroscopy Using Anti-PA(S) Mab 1.1 to Capture a PASylated Anti-Galectin Fab Fragment and to Determine the PAS-Fab Binding Kinetics Towards its Antigen Galectin-3

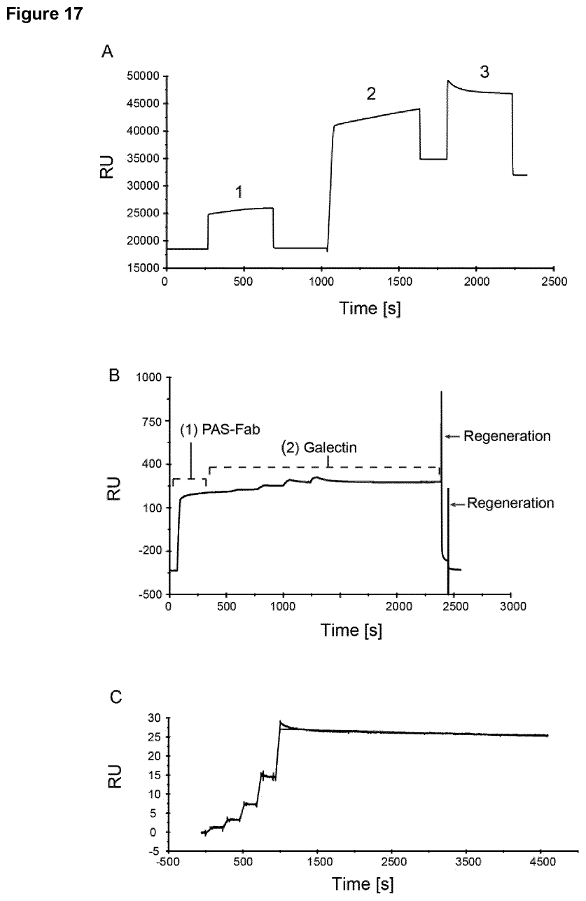

The anti-PA(S) Mab 1.1 antibody of this invention was used as a tool for the stable non-covalent capturing of a PASylated humanized anti-Galectin Fab fragment (Peplau et al., 2021) on a surface plasmon resonance (SPR) sensor chip to determine the affinity of this Fab to its antigen Galectin-3. A Biacore X100 instrument (Cytiva, Freiburg, Germany), operated with HBS/T (10 mM HEPES pH 7.4, 150 mM NaCl, 3 mM EDTA, 0.005% v/v Tween 20) as running buffer at a flow rate of 30 μl/min, was charged with a carboxymethyl dextran-coated CM5 sensor chip (Cytiva). The carboxylate groups of the dextran hydrogel in both flow channels were converted to reactive N-hydroxysuccinimide ester groups using an amine coupling kit (Cytiva) by injecting a 1:1 mixture of 483 mM 1-ethyl-3-(3-dimethylaminopropyl) carbodiimide hydrochloride (EDC) and 100 mM N-hydroxysuccinimide (NHS) for 430 s at a flow rate of 5 μl/min. Next, the protein A affinity purified recombinant anti-PA(S) Mab 1.1 obtained from Genscript (Piscataway, NJ, USA) was covalently immobilized onto the chip surface by injection of a 100 μg/ml anti-PA(S) Mab 1.1 solution in 10 mM Na-acetate pH 4.5 for 600 s at a flow rate of 5 μl/min. Unreacted NHS ester groups were finally blocked by injection of an 0.1 M ethanolamine solution for 430 s at a flow rate of 5 μl/min. This procedure (FIG. 17) resulted in an anti-PA(S) Mab 1.1 surface density of approximately 13500 resonance units (ΔRU).

To investigate the binding kinetics of the anti-Galectin-PAS(200) Fab fragment (SEQ ID NO: 92 and SEQ ID NO: 93) towards recombinant Galectin-3 (Uniprot Identifier P17931) carrying a C173T mutation and a C-terminal Strep-tag II (SEQ ID NO: 94), the purified PASylated Fab fragment was diluted in HBS/T to 3.57 μg/ml and injected into flow channel 2 for 40 s at a flow rate of 5 μl/min, followed by buffer flow for 600 s. This resulted in a PAS-Fab surface density of approximately 580 resonance units (ARU), which remained stable within ±7% (FIG. 17B). Subsequently, a single cycle kinetic experiment was performed using five consecutive injections from a 1:2 dilution series (1.6 nM to 0.1 nM) of the antigen Galectin-3 at a flow rate of 30 μL/min, each for 60 s, finally followed by a 3600 s dissociation period. After that, the chip was regenerated by two subsequent injections of 10 mM glycine/HCl pH 2.4 for 60 s at a flow rate of 30 μl/min. This regeneration procedure allowed reuse of the same MAb-functionalized sensor surface for further measurements with PASylated proteins over more than two month.

The reference-corrected sensorgram (FIG. 17C) showed binding curves typical for a bimolecular reaction between the anti-Galectin-PAS(200) Fab fragment and its antigen Galectin-3. These data were fitted to a global 1:1 Langmuir binding model using Biacore X100 evaluation software (Cytiva), resulting in an association rate of 7.9×10⁶ M⁻¹ s⁻¹, a dissociation rate of 3.0×10⁻⁵ s⁻¹ and an equilibrium dissociation constant (K_{D} value) of 3.8 pM.

These findings demonstrate that the highly specific anti-PAS 1.1 MAb of this invention offers a valuable tool for the stable non-covalent capturing of a PASylated protein on an SPR sensor chip. Furthermore, mild acidic regeneration using 10 mM glycine/HCl pH 2.4 completely removed the PASylated protein, together with its ligand, offering the ability to reuse the same MAb-functionalized sensor surface for further measurements with PASylated proteins.

### REFERENCES

Al-Lazikani, B., et al. (1997) J Mol Biol 273, 927-948.
Baker, N. A., et al. (2001) Proc Natl Acad Sci USA 9, 10037-10041.
Barlow, D. J., et al. (1986) Nature 322 747-748.
Binder, U. & Skerra, A. (2017) Curr Opin Colloid Interface Sci, 10-17.
Birtalan, S., et al. (2008) J Mol Biol 377, 1518-1528.
Birtalan, S., et al. (2010) Mol Biosyst 6, 1186-1194.
Bolze, F., et al. (2016) Endocrinology 157, 233-244.
Breibeck, J. & Skerra, A. (2018) Biopolymers 109.
Chardes, T., et al. (1999) FEBS Lett 452 386-394.
Chothia, C., et al. (1989) Nature 342, 877-883.
Clackson, T. & Wells, J. A. (1995) Science 267, 383-386.
Cunningham, B. C. & Wells, J. A. (1989) Science 24, 1081-1085.
Edwardraja, S., et al. (2017) ACS Synth Biol 6, 2241-2247.
Emsley, P., et al. (2010) Acta Crystallogr D Biol Crystallogr 66, 486-501.
Ereno-Orbea, J., et al. (2018) J Mol Biol 430, 322-336.
Fassolari, M., et al. (2013) J Biol Chem 288, 13110-13123.
Feng, Z. P., et al. (2006) Mol Biochem Parasitol 150, 256-267.
Fling, S. P. & Gregerson, D. S. (1986) Anal Biochem 155 83-88.
Fong, J. H., et al. (2009) PLoS Comput Biol 5, e1000316.
Frank, R. (2002) J Immunol Methods 267 13-26.
Frishman, D. & Argos, P. (1995) Proteins 23 566-579.
Gao, J., et al. (2009) Nat Struct Mol Biol 1, 684-690.
Gasteiger, E., et al. (2003) Nucleic Acids Res 31 3784-3788.
Gebauer, M. & Skerra, A. (2018) Bioorg Med Chem 6, 2882-2887.
Giri, R., et al. (2016) Front Cell Infect Microbiol 6, 144.
Goh, G. K., et al. (2016) Mol Biosyst 1, 1881-1891.
Griffiths, K., et al. (2019) MAbs 11 1331-1340.
Harari, D., et al. (2014) J Biol Chem 289, 29014-29029.
Harlow, E. & Lane, D. (1988) Antibodies: A Laboratory Manual. Cold Spring Harbor Laboratory Press, Cold Spring Harbor, NY.
Huang, J. & Honda, W. (2006) BMC Immunol 7 7.
Kabat, E. A., et al. (1991) Sequences of Proteins of Immunological Interest. 5th ed. National Institute of Health, Bethesda, MD.
Kabsch, W. (2010) Acta Crystallogr D Biol Crystallogr 66, 125-132.
Karlsson, R., et al. (2006) Anal Biochem 349, 136-147.
Krissinel, E. & Henrick, K. (2007) J Mol Biol 372, 774-797.
Langin, M., et al. (2018) Nature 564, 430-433.
Loers, G., et al. (2014) Biochem J 460, 437-446.
London, N., et al. (2010) Structure 8, 188-199.
MacRaild, C. A., et al. (2016) Structure 24, 148-157.
McCoy, A. J., et al. (2007) J Appl Crystallogr 40, 658-674.
Meerman, H. J. & Georgiou, G. (1994) Bio/Technology, 1107-1110.
Mendler, C. T., et al. (2015) MAbs 7, 96-109.
Meszaros, B., et al. (2007) J Mol Biol 372 549-561.
Morath, V., et al. (2015) Mol Pharm 12 1431-1442.
Murshudov, G. N., et al. (2011) Acta Crystallogr D Biol Crystallogr 67, 355-367.
Peplau, E., et al. (2020) Thyroid 3, 1314-1326.
Peplau, E., et al. (2021) Sci Rep 11, 7358.
Richter, A., et al. (2020) Nuc/Med MolImaging 54, 114-119.
Schellenberger, V., et al. (2009) Nat Biotechnol 27 1186-1190.
Schiweck, W. & Skerra, A. (1995) Proteins 2, 561-565.
Schlapschy, M., et al. (2013) Protein Eng Des Sel 26, 489-501.
Skerra, A. (1994) Gene 141, 79-84.
Snead, D. & Eliezer, D. (2019) J Biol Chem 294, 3325-3342.
Suzuki, M. & Yagi, N. (1991) Proc Biol Sci 246, 231-235.
Swaminathan, A., et al. (2014) Br J Clin Pharmacol 78, 1135-1142.
Tantos, A., et al. (2012) Mol Cell Endocrinol 348, 457-465.
Uversky, V. N. (2019) Front Phys 7 10.
Vashist, S. K. & Luong, J. H. T. (2018) Handbook of Immunoassay Technologies—Approaches, Performances, and Applications. 1st ed. Academic Press, London, UK.
Voss, S. & Skerra, A. (1997) Protein Eng 10, 975-982.
Williams, C. J., et al. (2018) Protein Sci 27 293-315.
Winn, M. D., et al. (2011) Acta Crystallogr D Biol Crystallogr 7, 235-242.
Wright, P. E. & Dyson, H. J. (2015) Nat Rev Mol Cell Biol 1, 18-29.
Yanisch-Perron, C., et al. (1985) Gene 33, 103-119.
Zander, H., et al. (2007) J Mol Recognit 20, 185-196.

1. A method for generating an antigen binding molecule, preferably an antibody or an antigen-binding fragment thereof, directed against intrinsically disordered peptides/proteins and/or intrinsically disordered peptide/protein domains, said method comprising the step of immunizing a non-human mammal with an antigen
wherein said antigen is a conjugate of an immunoadjuvant and one or more P/A peptides,
wherein each P/A peptide is independently a peptide consisting of about 5 to about 100 amino acid residues, wherein at least 60% of the amino acid residues of said peptide are independently selected from proline and alanine, and wherein a protecting group R^{N} is attached to the N-terminal amino group of said peptide.

2. The method of claim 1, wherein each P/A peptide is independently a peptide
R^{N}—(P/A)-R^{C},
wherein (P/A) is an amino acid sequence consisting of about 8 to about 90 amino acid residues, wherein at least 70% of the number of amino acid residues in (P/A) are independently selected from proline and alanine, wherein (P/A) includes at least one proline residue and at least one alanine residue,
wherein R^{N} is a protecting group which is attached to the N-terminal amino group of (P/A),
wherein R^{C} is an amino acid residue which is bound via its amino group to the C-terminal carboxy group of (P/A) and which comprises at least one carbon atom between its amino group and its carboxy group, and
wherein each P/A peptide is conjugated to the immunoadjuvant via an amide linkage formed from the carboxy group of the C-terminal amino acid residue R^{C} of the P/A peptide and a free amino group of the immunoadjuvant.

3. The method of claim 2, wherein (P/A) in said antigen is an amino acid sequence consisting of about 10 to about 80 amino acid residues, wherein at least 70% of the number of amino acid residues in (P/A) are independently selected from proline and alanine, wherein at least 95% of the number of amino acid residues in (P/A) are independently selected from proline, alanine and serine, and wherein (P/A) includes at least one proline residue and at least one alanine residue.

4. The method of claim 2, wherein (P/A) in said antigen is an amino acid sequence consisting of 20 to 40 amino acid residues independently selected from proline, alanine and serine, wherein at least 70% of the number of amino acid residues in (P/A) are independently selected from proline and alanine, and wherein (P/A) includes at least one proline residue and at least one alanine residue.

5. The method of claim 2, wherein the proportion of the number of proline residues comprised in (P/A) to the total number of amino acid residues comprised in (P/A) is ≥10% and ≤70%, preferably ≥20% and ≤50%, more preferably ≥25% and ≤40%.

6. The method of claim 2, wherein (P/A) in said antigen consists of (i) five or more partial sequences independently selected from ASPA (SEQ ID NO: 86), APAP (SEQ ID NO: 87), SAPA (SEQ ID NO: 88), AAPA (SEQ ID NO: 89) and APSA (SEQ ID NO: 84), and (ii) optionally one, two or three further amino acid residues independently selected from proline, alanine and serine.

7. The method of claim 2, wherein (P/A) consists of (i) the sequence ASPA-APAP-ASPA-APAP-SAPA, (ii) the sequence AAPA-APAP-AAPA-APAP-AAPA, (iii) the sequence APSA-APSA-APSA-APSA-APSA, (iv) a duplication of any of the aforementioned sequences, or (v) a combination of two of the aforementioned sequences.

8. The method of claim 1, wherein R^{N} is selected from pyroglutamoyl (Pga), homopyroglutamoyl, formyl, acetyl, hydroxyacetyl, methoxyacetyl, ethoxyacetyl, propoxyacetyl, propionyl, 2-hydroxypropionyl, 3-hydroxypropionyl, 2-methoxypropionyl, 3-methoxypropionyl, 2-ethoxypropionyl, 3-ethoxypropionyl, butyryl, 2-hydroxybutyryl, 3-hydroxybutyryl, 4-hydroxybutyryl, 2-methoxybutyryl, 3-methoxybutyryl, 4-methoxybutyryl, glycine betainyl, o-aminobenzoyl, —NH—(C₁₋₆ alkyl), —N,N(C₁₋₈ alkyl)₂, N,N,N-tri(C₁₋₆-alkyl)₃, N,N-tetramethylene, and N,N-pentamethylene.

9. The method of claim 2, wherein R^{C} is H₂N—(C₁₋₁₂ hydrocarbyl)-COOH, wherein it is preferred that R^{C} is selected from H₂N—(CH₂)₁₋₁₀—COOH, H₂N-phenyl-COOH, and H₂N-cyclohexyl-COOH, wherein it is more preferred that R^{C} is selected from H₂N—CH₂—COOH (Gly), H₂N—(CH₂)₂—COOH (D-Ala), H₂N—(CH₂)₃—COOH, H₂N—(CH₂)₄—COOH, H₂N—(CH₂)₅—COOH, H₂N—(CH₂)₆—COOH, H₂N—(CH₂)₇—COOH, H₂N—(CH₂)₈—COOH, p-aminobenzoic acid, and 4-aminocyclohexanecarboxylic acid, and wherein it is even more preferred that R^{C} is H₂N—(CH₂)₅—COOH.

10. The method of claim 1, wherein the P/A peptide(s) comprised in said antigen adopt(s) a random coil conformation and/or wherein the P/A peptide(s) comprised in said antigen is/are devoid of charged residues.

11. The method of claim 1, wherein the immunoadjuvant is selected from keyhole limpet hemocyanin (KLH), ovalbumin (OVA), and bovine serum albumin (BSA), preferably wherein the immunoadjuvant is keyhole limpet hemocyanin (KLH).

12. An antigen as defined in claim 1.

13. (canceled)

14. The method of claim 1, wherein said intrinsically disordered peptides/proteins and/or intrinsically disordered peptide/protein domains are Pro/Ala-rich sequences,
preferably wherein said Pro/Ala-rich sequences are amino acid sequences consisting of at least 20 amino acid residues forming random coil conformation and whereby said amino acid residues forming said random coil conformation are selected from Pro (P), Ala (A) and Ser (S), preferably from Pro (P) and Ala (A).

15. The method of claim 14, wherein said Pro/Ala-rich sequences comprise at least one epitope of the structure
(P/S)A(A/S)P; and/or
PA(A/S)P;
preferably wherein said epitope comprises an epitope stretch selected from the group consisting of PAAP, PASP, PAPASP, PAPAAP, PASPAAP, and APSA.

16. An antigen binding molecule, preferably an antibody or an antigen-binding fragment thereof, directed against intrinsically disordered peptides/proteins and/or intrinsically disordered peptide/protein domains, which is obtainable by the method of claim 1.

17. An antigen-binding molecule, wherein said antigen-binding molecule is selected from the group consisting of:
a) an antibody or an antigen-binding fragment thereof, comprising
a variable heavy (VH) chain comprising
the CDR-H1 as defined in SEQ ID NO: 35 [anti-PA(S) MAb 1.1],
the CDR-H2 as defined in SEQ ID NO: 36 [anti-PA(S) MAb 1.1], and
the CDR-H3 as defined in SEQ ID NO: 37 [anti-PA(S) MAb 1.1]; and/or
a variable light (VL) chain comprising
the CDR-L1 as defined in SEQ ID NO: 38 [anti-PA(S) MAb 1.1],
the CDR-L2 as defined in SEQ ID NO: 39 [anti-PA(S) MAb 1.1], and
the CDR-L3 as defined in SEQ ID NO: 40 [anti-PA(S) MAb 1.1]; or
is an antibody or an antigen-binding fragment thereof binding to the same epitope as an antibody comprising any one or more of the CDRs of (a);
b) an antibody or an antigen-binding fragment thereof, comprising
a variable heavy (VH) chain comprising
the CDR-H1 as defined in SEQ ID NO: 41 [anti-PA(S) MAb 1.2],
the CDR-H2 as defined in SEQ ID NO: 42 [anti-PA(S) MAb 1.2], and
the CDR-H3 as defined in SEQ ID NO: 43 [anti-PA(S) MAb 1.2]; and/or
a variable light (VL) chain comprising
the CDR-L1 as defined in SEQ ID NO: 44 [anti-PA(S) MAb 1.2],
the CDR-L2 as defined in SEQ ID NO: 45 [anti-PA(S) MAb 1.2], and
the CDR-L3 as defined in SEQ ID NO: 46 [anti-PA(S) MAb 1.2]; or
is an antibody or an antigen-binding fragment thereof binding to the same epitope as an antibody comprising any one or more of the CDRs of (b);
c) an antibody or an antigen-binding fragment thereof, comprising
a variable heavy (VH) chain comprising
the CDR-H1 as defined in SEQ ID NO: 47 [anti-PA(S) MAb 2.1],
the CDR-H2 as defined in SEQ ID NO: 48 [anti-PA(S) MAb 2.1], and
the CDR-H3 as defined in SEQ ID NO: 49 [anti-PA(S) MAb 2.1]; and/or
a variable light (VL) chain comprising
the CDR-L1 as defined in SEQ ID NO: 50 [anti-PA(S) MAb 2.1],
the CDR-L2 as defined in SEQ ID NO: 51 [anti-PA(S) MAb 2.1], and
the CDR-L3 as defined in SEQ ID NO: 52 [anti-PA(S) MAb 2.1]; or
is an antibody or an antigen-binding fragment thereof binding to the same epitope as an antibody comprising any one or more of the CDRs of (c);
d) an antibody or an antigen-binding fragment thereof, comprising
a variable heavy (VH) chain comprising
the CDR-H1 as defined in SEQ ID NO: 53 [anti-PA(S) MAb 2.2],
the CDR-H2 as defined in SEQ ID NO: 54 [anti-PA(S) MAb 2.2], and
the CDR-H3 as defined in SEQ ID NO: 55 [anti-PA(S) MAb 2.2]; and/or
a variable light (VL) chain comprising
the CDR-L1 as defined in SEQ ID NO: 56 [anti-PA(S) MAb 2.2],
the CDR-L2 as defined in SEQ ID NO: 57 [anti-PA(S) MAb 2.2], and
the CDR-L3 as defined in SEQ ID NO: 58 [anti-PA(S) MAb 2.2]; or
is an antibody or an antigen-binding fragment thereof binding to the same epitope as an antibody comprising any one or more of the CDRs of (d);
e) an antibody or an antigen-binding fragment thereof, comprising
a variable heavy (VH) chain comprising
the CDR-H1 as defined in SEQ ID NO: 59 [anti-PA(S) MAb 3.1],
the CDR-H2 as defined in SEQ ID NO: 60 [anti-PA(S) MAb 3.1], and
the CDR-H3 comprising or consisting of the amino acid sequence Trp-Gly-Arg [anti-PA(S) MAb 3.1]; and/or
a variable light (VL) chain comprising
the CDR1-L as defined in SEQ ID NO: 62 [anti-PA(S) MAb 3.1],
the CDR2-L as defined in SEQ ID NO: 63 [anti-PA(S) MAb 3.1], and
the CDR3-L as defined in SEQ ID NO: 64 [anti-PA(S) MAb 3.1]; or
is an antibody or an antigen-binding fragment thereof binding to the same epitope as an antibody comprising any one or more of the CDRs of (e); and
f) an antibody or an antigen-binding fragment thereof, comprising
a variable heavy (VH) chain comprising
the CDR-H1 as defined in SEQ ID NO: 65 [anti-PA(S) MAb 3.2],
the CDR-H2 as defined in SEQ ID NO: 66 [anti-PA(S) MAb 3.2], and
the CDR-H3 as defined in SEQ ID NO: 67 [anti-PA(S) MAb 3.2]; and/or
a variable light (VL) chain comprising
the CDR-L1 as defined in SEQ ID NO: 68 [anti-PA(S) MAb 3.2],
the CDR-L2 as defined in SEQ ID NO: 69 [anti-PA(S) MAb 3.2], and
the CDR-L3 as defined in SEQ ID NO: 70 [anti-PA(S) MAb 3.2]; or
is an antibody or an antigen-binding fragment thereof binding to the same epitope as an antibody comprising any one or more of the CDRs of (f)

18. The antigen-binding molecule of claim 17, wherein said antigen-binding molecule is an antibody or an antigen-binding fragment thereof, which:
a) comprises a variable heavy (VH) chain sequence comprising the amino acid sequence of SEQ ID NO: 11 [anti-PA(S) MAb 1.1], SEQ ID NO: 13 [anti-PA(S) MAb 1.2], SEQ ID NO: 15 [anti-PA(S) MAb 2.1], SEQ ID NO: 17 [anti-PA(S) MAb 2.2], SEQ ID NO: 19 [anti-PA(S) MAb 3.1] or SEQ ID NO: 21 [anti-PA(S) MAb 3.2],
or a sequence having 85%, preferably 87%, more preferably at least 90% sequence identity to SEQ ID NO: 11, 13, 15, 17, 19 or 21; and/or
comprises a variable light (VL) chain sequence comprising the amino acid sequence of SEQ ID NO: 12 [anti-PA(S) MAb 1.1], SEQ ID NO: 14 [anti-PA(S) MAb 1.2], SEQ ID NO: 16 [anti-PA(S) MAb 2.1], SEQ ID NO: 18 [anti-PA(S) MAb 2.2], SEQ ID NO: 20 [anti-PA(S) MAb 3.1] or SEQ ID NO: 22 [anti-PA(S) MAb 3.2],
or a sequence having 85%, preferably 87%, more preferably at least 90% sequence identity to SEQ ID NO: 12, 14, 16, 18, 20 or 22; or
b) is an antibody or an antigen-binding fragment thereof binding to the same epitope as an antibody of (a).

19. The antigen-binding molecule of claim 17, wherein the antigen-binding molecule is an antigen-binding fragment selected from a Fab fragment, a F(ab′)₂ fragment, a Fv fragment or a scFv fragment.

20. The antigen-binding molecule of claim 17, wherein the antigen-binding molecule is conjugated or fused to a reporter molecule and/or a label.

21. The antigen-binding molecule of claim 17, wherein the antigen-binding molecule is employed in matrix-based protein/peptide purification or immobilization.

22. The antigen-binding molecule of claim 17, wherein said antigen-binding molecule is selected from the group consisting of:
a) an antibody or an antigen-binding fragment thereof, comprising
a variable heavy (VH) chain comprising
the CDR-H1 as defined in SEQ ID NO: 35 [anti-PA(S) MAb 1.1],
the CDR-H2 as defined in SEQ ID NO: 36 [anti-PA(S) MAb 1.1], and
the CDR-H3 as defined in SEQ ID NO: 37 [anti-PA(S) MAb 1.1]; and/or
a variable light (VL) chain comprising
the CDR-L1 as defined in SEQ ID NO: 38 [anti-PA(S) MAb 1.1],
the CDR-L2 as defined in SEQ ID NO: 39 [anti-PA(S) MAb 1.1], and
the CDR-L3 as defined in SEQ ID NO: 40 [anti-PA(S) MAb 1.1];
b) an antibody or an antigen-binding fragment thereof, comprising
a variable heavy (VH) chain comprising
the CDR-H1 as defined in SEQ ID NO: 41 [anti-PA(S) MAb 1.2],
the CDR-H2 as defined in SEQ ID NO: 42 [anti-PA(S) MAb 1.2], and
the CDR-H3 as defined in SEQ ID NO: 43 [anti-PA(S) MAb 1.2]; and/or
a variable light (VL) chain comprising
the CDR-L1 as defined in SEQ ID NO: 44 [anti-PA(S) MAb 1.2],
the CDR-L2 as defined in SEQ ID NO: 45 [anti-PA(S) MAb 1.2], and
the CDR-L3 as defined in SEQ ID NO: 46 [anti-PA(S) MAb 1.2];
c) an antibody or an antigen-binding fragment thereof, comprising
a variable heavy (VH) chain comprising
the CDR-H1 as defined in SEQ ID NO: 47 [anti-PA(S) MAb 2.1],
the CDR-H2 as defined in SEQ ID NO: 48 [anti-PA(S) MAb 2.1], and
the CDR-H3 as defined in SEQ ID NO: 49 [anti-PA(S) MAb 2.1]; and/or
a variable light (VL) chain comprising
the CDR-L1 as defined in SEQ ID NO: 50 [anti-PA(S) MAb 2.1],
the CDR-L2 as defined in SEQ ID NO: 51 [anti-PA(S) MAb 2.1], and
the CDR-L3 as defined in SEQ ID NO: 52 [anti-PA(S) MAb 2.1];
d) an antibody or an antigen-binding fragment thereof, comprising
a variable heavy (VH) chain comprising
the CDR-H1 as defined in SEQ ID NO: 53 [anti-PA(S) MAb 2.2],
the CDR-H2 as defined in SEQ ID NO: 54 [anti-PA(S) MAb 2.2], and
the CDR-H3 as defined in SEQ ID NO: 55 [anti-PA(S) MAb 2.2]; and/or
a variable light (VL) chain comprising
the CDR-L1 as defined in SEQ ID NO: 56 [anti-PA(S) MAb 2.2],
the CDR-L2 as defined in SEQ ID NO: 57 [anti-PA(S) MAb 2.2], and
the CDR-L3 as defined in SEQ ID NO: 58 [anti-PA(S) MAb 2.2];
e) an antibody or an antigen-binding fragment thereof, comprising
a variable heavy (VH) chain comprising
the CDR-H1 as defined in SEQ ID NO: 59 [anti-PA(S) MAb 3.1],
the CDR-H2 as defined in SEQ ID NO: 60 [anti-PA(S) MAb 3.1], and
the CDR-H3 comprising or consisting of the amino acid sequence Trp-Gly-Arg [anti-PA(S) MAb 3.1]; and/or
a variable light (VL) chain comprising
the CDR1-L as defined in SEQ ID NO: 62 [anti-PA(S) MAb 3.1],
the CDR2-L as defined in SEQ ID NO: 63 [anti-PA(S) MAb 3.1], and
the CDR3-L as defined in SEQ ID NO: 64 [anti-PA(S) MAb 3.1]; or and
f) an antibody or an antigen-binding fragment thereof, comprising
a variable heavy (VH) chain comprising
the CDR-H1 as defined in SEQ ID NO: 65 [anti-PA(S) MAb 3.2],
the CDR-H2 as defined in SEQ ID NO: 66 [anti-PA(S) MAb 3.2], and
the CDR-H3 as defined in SEQ ID NO: 67 [anti-PA(S) MAb 3.2]; and/or
a variable light (VL) chain comprising
the CDR-L1 as defined in SEQ ID NO: 68 [anti-PA(S) MAb 3.2],
the CDR-L2 as defined in SEQ ID NO: 69 [anti-PA(S) MAb 3.2], and
the CDR-L3 as defined in SEQ ID NO: 70 [anti-PA(S) MAb 3.2]; or

23. The antigen-binding molecule of claim 17 wherein said antigen-binding molecule is an antibody or an antigen-binding fragment thereof, which:
comprises a variable heavy (VH) chain sequence comprising the amino acid sequence selected from the group consisting of SEQ ID NO: 11 [anti-PA(S) MAb 1.1], SEQ ID NO: 13 [anti-PA(S) MAb 1.2], SEQ ID NO: 15 [anti-PA(S) MAb 2.1], SEQ ID NO: 17 [anti-PA(S) MAb 2.2], SEQ ID NO: 19 [anti-PA(S) MAb 3.1] and SEQ ID NO: 21 [anti-PA(S) MAb 3.2], or
comprises a variable light (VL) chain sequence comprising the amino acid sequence selected from the group consisting of SEQ ID NO: 12 [anti-PA(S) MAb 1.1], SEQ ID NO: 14 [anti-PA(S) MAb 1.2], SEQ ID NO: 16 [anti-PA(S) MAb 2.1], SEQ ID NO: 18 [anti-PA(S) MAb 2.2], SEQ ID NO: 20 [anti-PA(S) MAb 3.1] or SEQ ID NO: 22 [anti-PA(S) MAb 3.2].
